(19) United States
(12) Patent Application Publication
(10) Pub. No.: US 2012/0202772 A1
(43) Pub. Date: Aug. 9, 2012

(54) CYCLODEXTRIN-BASED POLYMERS FOR THERAPEUTIC DELIVERY

(76) Inventors: Alexandra Glucksmann, Lexington, MA (US); Lawrence Alan Reiter, Mystic, CT (US); Thomas C. Crawford, Essex, CT (US)

(21) Appl. No.: 13/448,910

(22) Filed: Apr. 17, 2012

Related U.S. Application Data
(63) Continuation of application No. 13/209,202, filed on Aug. 12, 2011, now abandoned.
Application No. 13/209,202 is a continuation-in-part of application No. 13/008,265, filed on Jan. 18, 2011, now abandoned.
(60) Provisional application No. 61/296,126, filed on Jan. 19, 2010.
(60) Provisional application No. 61/296,690, filed on Jan. 20, 2010.

(51) Int. Cl. A61K 31/724; A61P 35/00; C08B 37/16; B82Y 5/00

(52) U.S. Cl. 514/58; 536/103; 977/773; 977/915

(57) ABSTRACT

Methods and compositions relating to CDP-proteasome inhibitor conjugates are described herein.

### CROSS-REFERENCE TO RELATED PATENT APPLICATIONS

This application is a continuation-in-part of U.S. Application No. 13/008,265, filed Jan. 18, 2011, which claims the benefit of U.S. Provisional Application No. 61/296,126, filed Jan. 19, 2010, and U.S. Provisional Application No. 61/296,690, filed Jan. 20, 2010, the contents of each of which is incorporated herein by reference in its entirety.

### BACKGROUND OF THE INVENTION

Drug delivery of some small molecule therapeutic agents, such as proteasome inhibitors, has been problematic due to their poor pharmacological profiles. These therapeutic agents often have low aqueous solubility, their bioactive forms exist in equilibrium with an inactive form, or high systemic concentrations of the agents lead to toxic side-effects. Some approaches to circumvent the problem of their delivery have been to conjugate the agent directly to a water-soluble polymer such as hydroxypropyl methacrylate (HPMA), polyethyleneglycol, and poly-L-glutamic acid. In some cases, such conjugates have been successful in solubilizing or stabilizing the bioactive form of the therapeutic agent, or achieving a sustained release formulation which circumvents complications associated with high systemic concentrations of the agent.

Another approach to the drug delivery problem has been to form host/guest inclusion complexes between the therapeutic agent and cyclodextrins or derivatives thereof. Cyclodextrins (alpha, beta, and gamma) and their oxidized forms have unique physico-chemical properties such as good water solubility, low toxicity and low immune response. To date, most of the drug delivery studies with cyclodextrins have focused on their ability to form supra-molecular complexes, wherein cyclodextrins form host/guest inclusion complexes with therapeutic molecules and thus alter the physical, chemical, and/or biological properties of these guest molecules.

### SUMMARY OF THE INVENTION

In one aspect, the disclosure features a cyclodextrin containing polymer (CDP)-proteasome inhibitor (such as a boronic acid containing proteasome inhibitor) conjugate, e.g., a CDP-bortezomib conjugate described herein, and methods of making the CDP-proteasome inhibitor (such as a boronic acid containing proteasome inhibitor) conjugates, e.g., a CDP-bortezomib conjugates, described herein.

In one embodiment, CDP is not biodegradable.

In one embodiment, CDP is biocompatible.

In one embodiment, the CDP-proteasome inhibitor (such as a boronic acid containing proteasome inhibitor) conjugate, e.g., a CDP-bortezomib conjugate, forms a nanoparticle, wherein the nanoparticle includes an inclusion complex between a proteasome inhibitor (such as a boronic acid containing proteasome inhibitor), e.g., bortezomib, attached or conjugated to the CDP, e.g., via a covalent linkage, and another molecule in the CDP. In one embodiment, the CDP-proteasome inhibitor (such as a boronic acid containing proteasome inhibitor) conjugate, e.g., a CDP-bortezomib conjugate, forms a nanoparticle. In one embodiment, the CDP-proteasome inhibitor (such as a boronic acid containing proteasome inhibitor) conjugate, e.g., a CDP-bortezomib conjugate, including an inclusion complex forms a nanoparticle. The nanoparticle ranges in size from 10 to 300 nm in diameter, e.g., 20 to 280, 30 to 250, 30 to 200, 20 to 150, 30 to 100, 20 to 80, 30 to 70, 30 to 60, 30 to 50, 20 to 50, or 20 to 40 nm diameter. In one embodiment, the nanoparticle is 30 to 60 nm in diameter. In one embodiment, the composition comprises a population or a plurality of nanoparticles with an average diameter from 10 to 300 nm, e.g., 20 to 280, 30 to 250, 30 to 200, 20 to 150, to 100, 20 to 80, 30 to 70, 30 to 60, 30 to 50, 20 to 50 or 20 to 40 nm. In one embodiment, the average nanoparticle diameter is from 30 to 60 nm. In one embodiment, the surface charge of the molecule is neutral, or slightly negative. In some embodiments, the zeta potential of the particle surface is from about −80 mV to about 50 mV, about −20 mV to about 20 mV, about −20 mV to about −10 mV, or about −10 mV to about 0.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate), forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one embodiment, the proteasome inhibitor (such as a boronic acid containing proteasome inhibitor, e.g., bortezomib), conjugated to the CDP is more soluble when conjugated to the CDP, than when not conjugated to the CDP.

In one embodiment, the composition comprises a population, mixture or plurality of CDP-proteasome inhibitor (such as a boronic acid containing proteasome inhibitor) conjugates, e.g., CDP-bortezomib conjugates. In one embodiment, the population, mixture or plurality of CDP-proteasome inhibitor (such as a boronic acid containing proteasome inhibitor) conjugates comprise a plurality of different proteasome inhibitors (such as a boronic acid containing proteasome inhibitor) conjugated to a CDP (e.g., two different proteasome inhibitors (such as two different boronic acid containing proteasome inhibitors, e.g., bortezomib and N-(4-morpholine)carbonyl-β-(1-naphthyl)-L-alanine-L-leucine boronic acid; or a boronic acid containing proteasome inhibitor, e.g., bortezomib and a non-boronic acid containing proteasome inhibitor, e.g., lactacystin; or two different non-boronic acid containing proteasome inhibitors, e.g., lactacystin and epoxomycin) are in the composition such that two different proteasome inhibitors (such as two different boronic acid containing proteasome inhibitor, e.g., bortezomib and N-(4-morpholine)carbonyl-β-(1-naphthyl)-L-alanine-L-leucine boronic acid; or a boronic acid containing proteasome inhibitor, e.g., bortezomib and a non-boronic acid containing proteasome inhibitor, e.g., lactacystin; or two different non-boronic acid containing proteasome inhibitors, e.g., lactacystin and epoxomycin) are attached to a single CDP; or a first proteasome inhibitor (such as a boronic acid containing proteasome inhibitor, e.g., bortezomib, or a non-boronic acid containing proteasome inhibitor, e.g., lactacystin) is attached to a first CDP and a second proteasome inhibitor (such as a boronic acid containing proteasome inhibitor, e.g., N-(4-morpholine)carbonyl-β-(1-naphthyl)-L-alanine-L-leucine boronic acid, or a non-boronic acid containing proteasome inhibitor, e.g., epoxomycin) is attached to a second CDP and both CDP-proteasome inhibitor conjugates are present in the composition). In one embodiment, the population, mixture or plurality of CDP-proteasome inhibitor (such as a boronic acid containing proteasome inhibitor) conjugates comprises a CDP having a single proteasome inhibitor (such as a boronic acid containing proteasome inhibitor, e.g., bortezomib) attached thereto in a plurality of positions (e.g., a CDP has a single proteasome inhibitor (such as a boronic acid containing proteasome inhibitor, e.g., bortezomib) attached thereto such that the single proteasome inhibitor (such as a boronic acid containing proteasome inhibitor, e.g., bortezomib) for some occurrences is attached through a first position and for other occurrences is attached through a second position to thereby provide a CDP having single proteasome inhibitor (such as a boronic acid containing proteasome inhibitor, e.g., bortezomib) attached through a plurality of positions on the CDP).

In one aspect, the disclosure features a method of treating a proliferative disorder, e.g., a cancer (such as a solid tumor, a liquid tumor or a semi-solid tumor), in a subject, e.g., a human, the method comprises: administering a CDP-proteasome inhibitor (such as a boronic acid containing proteasome inhibitor) conjugate, particle (e.g., nanoparticle) or composition, e.g., a CDP-bortezomib conjugate, particle or composition, to a subject in an amount effective to treat the disorder, to thereby treat the proliferative disorder.

In an embodiment, the CDP-proteasome inhibitor (such as a boronic acid containing proteasome inhibitor, e.g., bortezomib) conjugate comprises a proteasome inhibitor molecule (such as a boronic acid containing proteasome inhibitor, e.g., bortezomib), coupled, e.g., via a linker, to a CDP described herein. In an embodiment, the CDP-proteasome inhibitor (such as a boronic acid containing proteasome inhibitor, e.g., bortezomib) conjugate comprises a proteasome inhibitor (such as a boronic acid containing proteasome inhibitor, e.g., bortezomib) molecule, coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In an embodiment, the method further comprises administering a chemotherapeutic agent as a free agent.

In an embodiment, the proteasome inhibitor (such as a boronic acid containing proteasome inhibitor, e.g., bortezomib) associated with the CDP and the free agent are the same anti-cancer agent. E.g., the agent is a proteasome inhibitor (such as a boronic acid containing proteasome inhibitor, e.g., bortezomib).

In an embodiment, the proteasome inhibitor (such as a boronic acid containing proteasome inhibitor, e.g., bortezomib) associated with the CDP and the free agent are different chemotherapeutic agents.

In one embodiment, the cancer is a cancer described herein, e.g., a solid tumor, a liquid tumor or a semi-solid tumor. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., unresectable, locally advanced or metastatic non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered by intravenous administration, e.g., an intravenous administration that is completed in a period equal to or less than 2 hours, 1.5 hours, 1 hour, 45 minutes or 30 minutes. In one embodiment, the composition is administered as a bolus infusion or intravenous push, e.g., over a period of 15 minutes, 10 minutes, 5 minutes or less. In one embodiment, the composition is administered as a bolus intravenous injection, e.g., over a period of 0-60 seconds, 1-30 seconds, 2-10 seconds or 3 to 5 seconds.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein is administered to the subject in an amount of 0.1-50 mg/m², 0.5-10 mg/m², or 1-5 mg/m². The dosage amount described herein is the amount of the proteasome inhibitor (such as a boronic acid containing proteasome inhibitor, e.g., bortezomib) in the CDP-proteasome inhibitor conjugate administered. In one embodiment, the CDP-proteasome inhibitor (such as a boronic acid containing proteasome inhibitor) conjugate is a CDP-bortezomib conjugate and the CDP-bortezomib conjugate is administered in the amount of 0.5-2 mg/m², 0.6-1.5 mg/m², 1.3-1.5 mg/m², 1.0-1.3 mg/m² or 0.7-1.0 mg/m². In one embodiment, the CDP-bortezomib conjugate is administered in the amount of 0.5, 0.6, 0.7, 0.8, 0.9, 1.0, 1.1, 1.2 or 1.3 mg/m². In another embodiment, the CDP-proteasome inhibitor (such as a boronic acid containing proteasome inhibitor) conjugate is a CDP-bortezomib conjugate described herein and the CDP-bortezomib conjugate is administered in the amount of 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3, 5, 3.6, 3.7, 3.8, 3.9 or 4.0 mg/m².

In one embodiment, the subject is administered at least one additional dose of the conjugate, e.g., the subject is administered, at least two, three, four, five, six, seven, eight, nine, ten, eleven or twelve additional doses of the conjugate. In one embodiment, the conjugate is administered once, twice, three or four times a week. In another embodiment, the conjugate is administered once or twice a week. In another embodiment, the conjugate is administered twice a week for two, three, four, five or six weeks followed by once a week for one, two, three, four, five or six weeks. In one embodiment, the conjugate is administered twice a week for six weeks followed by once a week for six weeks. In another embodiment, the conjugate is administered twice a week for two weeks followed by once a week for one, two, three or four weeks. In one embodiment, the conjugate is administered twice a week for two weeks followed by one or two weeks without administration of the conjugate. The subject can subsequently be administered once a week for one, two, three or four weeks. The dosage for the twice a week administration and once a week administration can be any dosage described above. In one embodiment, the dosage for twice a week administration and once a week administration are the same and can be any of the dosages described above. In one embodiment, the dosage for twice a week administration and once a week administration are different.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent that is preferably administered orally. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination melphalan and/or prednisone. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with melphalan and prednisone for treating a subject with multiple myeloma. More specifically, the melphalan and prednisone are administered orally.

In another aspect, the disclosure features a method of treating a chemotherapeutic sensitive, a chemotherapeutic refractory, a chemotherapeutic resistant, and/or a relapsed cancer. The method comprises: administering a composition comprising a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to a subject, e.g., a human, in an amount effective to treat the disorder, to thereby treat the cancer.

In an embodiment, the CDP-proteasome inhibitor (such as a boronic acid containing proteasome inhibitor) conjugate comprises a proteasome inhibitor molecule (such as a boronic acid containing proteasome inhibitor, e.g., a bortezomib), coupled, e.g., via linkers, to a CDP described herein. In an embodiment, the CDP-proteasome inhibitor (such as a boronic acid containing proteasome inhibitor) conjugate comprises a proteasome inhibitor molecule (such as a boronic acid containing proteasome inhibitor, e.g., a bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the cancer is refractory to, resistant to and/or relapsed during or after, treatment with, one or more of: an anthracycline (e.g., doxorubicin, daunorubicin, epirubicin, idarubicin, mitoxantrone, valrubicin), an alkylating agent (e.g., cyclophosphamide, dacarbazine, melphalan, ifosfamide, temozolomide), an antimetabolite (e.g., an antifolate, a purine analogue, a pyrimidine analogue (e.g., capecitabine)), a vinca alkaloid (e.g., vinblastine, vincristine, vindesine, vinorelbine), a topoisomerase inhibitor (e.g., topotecan, irinotecan, etoposide, teniposide, lamellarin D, SN-38, camptothecin (e.g., IT-101)) and a platinum-based agent (e.g., cisplatin, carboplatin, oxaliplatin). In one embodiment, the cancer is resistant to more than one chemotherapeutic agent, e.g., the cancer is a multidrug resistant cancer. In one embodiment, the cancer is resistant to one or more of a platinum based agent, an alkylating agent, an anthracycline and a vinca alkaloid. In one embodiment, the cancer is resistant to one or more of a platinum based agent, an alkylating agent, a taxane and a vinca alkaloid.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with a second chemotherapeutic agent, e.g., a chemotherapeutic agent described herein. For example, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, can be administered in combination with a vinca alkaloid (e.g., vinblastine, vincristine, vindesine, vinorelbine) and/or a platinum-based agent (e.g., cisplatin, carboplatin, oxaliplatin). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with melphalan and/or prednisone.

In one embodiment, the cancer is a cancer described herein, e.g., a solid tumor, a liquid tumor or a semi-solid tumor. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., unresectable, locally advanced or metastatic non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the composition includes a CDP-bortezomib conjugate, e.g., a CDP-bortezomib conjugate described herein, e.g., a CDP-bortezomib conjugate comprising bortezomib molecules, coupled, e.g., via linkers, to a CDP described herein.

In one embodiment, the CDP-bortezomib conjugate, particle (e.g., nanoparticle) or composition is administered at a dose and/or dosing schedule described herein.

In one aspect, the disclosure features a method of treating multiple myeloma in a subject, e.g., a human. The method comprises: administering a composition comprising a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to a subject in an amount effective to treat the myeloma, to thereby treat the myeloma.

In an embodiment, the CDP-proteasome inhibitor (such as a boronic acid containing proteasome inhibitor) conjugate comprises proteasome inhibitor, such as a boronic acid containing proteasome inhibitor (e.g., bortezomib), coupled, e.g., via linkers, to a CDP described herein. In an embodiment, the CDP-proteasome inhibitor (such as a boronic acid containing proteasome inhibitor) conjugate comprises a proteasome inhibitor, such as a boronic acid containing proteasome inhibitor described herein (e.g., bortezomib), coupled via a linker shown in Table 2 to a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate, particle (e.g., nanoparticle), composition, e.g., a CDP-bortezomib conjugate, particle (e.g., nanoparticle) or composition is administered as a primary treatment for multiple myeloma.

In one embodiment, the CDP-proteasome inhibitor (such as a boronic acid containing proteasome inhibitor) conjugate, particle (e.g., nanoparticle), composition, e.g., a CDP-bortezomib conjugate, particle (e.g., nanoparticle) or composition is administered in combination with dexamethasone. In one embodiment, the CDP-proteasome inhibitor (such as a boronic acid containing proteasome inhibitor) conjugate, particle (e.g., nanoparticle), composition, e.g., a CDP-bortezomib conjugate, particle (e.g., nanoparticle) or composition is further administered in combination with an anthracycline (e.g., daunorubicin, doxorubicin (e.g., liposomal doxorubicin), epirubicin, valrubicin and idarubicin), thalidomide or thalidomide derivative (e.g., lenalidomide). For example, in one embodiment, the CDP-proteasome inhibitor (such as a boronic acid containing proteasome inhibitor) conjugate, particle (e.g., nanoparticle) or composition is a CDP-bortezomib conjugate, particle (e.g., nanoparticle) or composition and the CDP-bortezomib conjugate, particle (e.g., nanoparticle) or composition is further administered in combination with an anthracycline (e.g., daunorubicin, doxorubicin (e.g., liposomal doxorubicin), epirubicin, valrubicin and idarubicin), thalidomide or thalidomide derivative (e.g., lenalidomide).

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with a vinca alkaloid (e.g., vinblastine, vincristine, vindesine and vinorelbine) and dexamethasone. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is further administered in combination with an anthracycline (e.g., daunorubicin, doxorubicin (e.g., liposomal doxorubicin), epirubicin, valrubicin and idarubicin). For example, in one embodiment, the CDP-proteasome inhibitor (such as a boronic acid containing proteasome inhibitor) conjugate, particle (e.g., nanoparticle) or composition is a CDP-bortezomib conjugate, particle (e.g., nanoparticle) or composition and the CDP-bortezomib conjugate, particle (e.g., nanoparticle) or composition is administered in combination with a vinca alkaloid (e.g., vinblastine, vincristine, vindesine and vinorelbine), dexamethasone, and an anthracycline (e.g., daunorubicin, doxorubicin (e.g., liposomal doxorubicin), epirubicin, valrubicin and idarubicin).

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with thalidomide or thalidomide derivative (e.g., lenalidomide). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is further administered in combination with dexamethasone.

In one embodiment, after the subject has received a primary treatment, e.g., a primary treatment described herein, the subject is further administered a high dose treatment. For example, the subject can be administered a high dose treatment of dexamethasone, an alkylating agent (e.g., cyclosposphamide or melphalan) and/or a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition described herein.

In one embodiment, after the primary treatment, e.g., after the primary treatment and the high dose treatment, stem cells are transplanted into the subject. In one embodiment, a subject who has received a stem cell transplant is administered thalidomide. In one embodiment, the subject is further administered a corticosteroid (e.g., prednisone).

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171.

In some embodiments, the composition is administered in combination with an mTOR inhibitor. Non-limiting examples of mTOR inhibitors include rapamycin, everolimus, AP23573, CCI-779 and SDZ-RAD.

In one embodiment, the CDP-proteasome inhibitor (such as a boronic acid containing proteasome inhibitor) conjugate is a CDP-bortezomib conjugate, e.g., a CDP-bortezomib conjugate, described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one aspect, the disclosure features a method of treating multiple myeloma in a subject, e.g., a human, the method comprising:

providing a subject who has multiple myeloma and has been treated with a chemotherapeutic agent that did not effectively treat the myeloma (e.g., the subject has a chemotherapeutic refractory myeloma, a chemotherapeutic resistant myeloma and/or a relapsed myeloma) or who had an unacceptable side effect (e.g., the subject has a chemotherapeutic sensitive myeloma), and

administering a CDP-proteasome inhibitor (such as a boronic acid containing proteasome inhibitor) conjugate, particle (e.g., nanoparticle) or composition, e.g., a CDP-bortezomib conjugate, particle (e.g., nanoparticle) or composition described herein, to a subject in an amount effective to treat the myeloma, to thereby treat the myeloma.

In an embodiment, the CDP-proteasome inhibitor (such as a boronic acid containing proteasome inhibitor) conjugate comprises a proteasome inhibitor, such as a boronic acid containing proteasome inhibitor (e.g., bortezomib), coupled, e.g., via linkers, to a CDP described herein. In an embodiment, the CDP-proteasome inhibitor (such as a boronic acid containing proteasome inhibitor) conjugate comprises a proteasome inhibitor, such as a boronic acid containing proteasome inhibitor (e.g., bortezomib), coupled via a linker shown in Table 2 to a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the subject has been treated with a proteasome inhibitor, e.g., bortezomib, which did not effectively treat the myeloma (e.g., the subject has a bortezomib refractory, a bortezomib resistant and/or relapsed myeloma).

In one embodiment, the subject has been treated with an anthracycline (e.g., daunorubicin, doxorubicin, epirubicin, valrubicin or idarubicin) which did not effectively treat the cancer (e.g., the subject has a doxorubicin refractory, a doxorubicin resistant and/or a relapsed myeloma).

In one embodiment, the subject has been treated with a thalidomide or thalidomide derivative (e.g., lenalidomide) which did not effectively treat the myeloma (e.g., the subject has thalidomide or thalidomide derivative refractory, thalidomide or thalidomide derivative resistant and/or a relapsed myeloma).

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is administered in combination with an anthracycline (e.g., daunorubicin, doxorubicin (e.g., liposomal doxorubicin), epirubicin, valrubicin and idarubicin). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is administered in combination with an anthracycline (e.g., daunorubicin, doxorubicin (e.g., liposomal doxorubicin), epirubicin, valrubicin and idarubicin).

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is administered in combination with thalidomide or a thalidomide derivative (e.g. lenalidomide) and dexamethasone.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is administered in combination with dexamethaxone and cyclophosphamide. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is further administered in combination with a topoisomerase inhibitor (e.g., etoposide, topotecan, irinotecan, tenoposide, SN-38, lamellarin D) and/or a platinum based agent (carboplatin, cisplatin, oxaliplatin). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is further administered in combination with an anthracycline (e.g., daunorubicin, doxorubicin (e.g., liposomal doxorubicin), epirubicin, valrubicin and idarubicin).

In an embodiment, the CDP-proteasome inhibitor (such as a boronic acid containing proteasome inhibitor) conjugate, e.g., a CDP-bortezomib conjugate, comprises a proteasome inhibitor (such as a boronic acid containing proteasome inhibitor, e.g., bortezomib) coupled, e.g., via linkers, to a polymer described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a polymer described herein. In an embodiment, the CDP-proteasome inhibitor conjugate is a CDP-proteasome inhibitor conjugate shown in Table 2.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition is administered at a dose and/or dosing schedule described herein.

In one aspect, the disclosure features a method of treating mantle cell lymphoma in a subject, e.g., a human. The method comprises: administering a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein (such as a to a CDP-bortezomib conjugate described herein) to a subject in an amount effective to treat the lymphoma, to thereby treat the lymphoma.

In an embodiment, the CDP-proteasome inhibitor (such as a boronic acid containing proteasome inhibitor) conjugate comprises a proteasome inhibitor (such as a boronic acid containing proteasome inhibitor, e.g., bortezomib) coupled, e.g., via linkers, to a CDP described herein. In an embodiment, the CDP-proteasome inhibitor conjugate comprises a proteasome inhibitor (such as a boronic acid containing proteasome inhibitor, e.g., bortezomib), coupled via a linker shown in Table 2 to a CDP described herein. In an embodiment, the CDP-proteasome inhibitor conjugate is a CDP-proteasome inhibitor conjugate shown in Table 2.

In one embodiment, the CDP-proteasome inhibitor (such as a boronic acid containing proteasome inhibitor) conjugate, particle (e.g., nanoparticle) or composition, e.g., a CDP-bortezomib conjugate, particle (e.g., nanoparticle) or composition, is administered in combination with an anthracycline (e.g., daunorubicin, doxorubicin (e.g., liposomal doxorubicin), epirubicin, valrubicin and idarubicin) and a vinca alkaloid (e.g., vinblastine, vincristine, vindesine and vinorelbine). For example, in one embodiment, the CDP-proteasome inhibitor (such as a boronic acid containing proteasome inhibitor) conjugate, particle (e.g., nanoparticle) or composition is a CDP-bortezomib conjugate, particle (e.g., nanoparticle) or composition and the CDP-bortezomib conjugate, particle (e.g., nanoparticle) or composition is further administered in combination with an anthracycline (e.g., doxorubicin (e.g., liposomal doxorubicin)) and a vinca alkaloid (e.g., vincristine). In one embodiment, the CDP-proteasome inhibitor (such as a boronic acid containing proteasome inhibitor) conjugate, particle (e.g., nanoparticle) or composition, e.g., a CDP-bortezomib conjugate, particle (e.g., nanoparticle) or composition, is further administered with one or more of an alkylating agent (e.g., cyclophosphamide, dacarbazine, melphalan, ifosfamide, temozolomide), prednisone, demethasone and rituximab. For example, in one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in one of the following combinations: an alkylating agent (e.g., cyclophosphamide), an anthracycline (e.g., doxorubicin (e.g., liposomal doxorubicin)), a vinca alkaloid (e.g., vincristine) and prednisone; an alkylating agent (e.g., cyclophosphamide), an anthracycline (e.g., doxorubicin (e.g., liposomal doxorubicin)), a vinca alkaloid (e.g., vincristine), prednisone and rituximab; an alkylating agent (e.g., cyclophosphamide), an anthracycline (e.g., doxorubicin (e.g., liposomal doxorubicin)), a vinca alkaloid (e.g., vincristine) and demethasone; an alkylating agent (e.g., cyclophosphamide), an anthracycline (e.g., doxorubicin (e.g., liposomal doxorubicin)), a vinca alkaloid (e.g., vincristine), demethasone and rituximab; an anthracycline (e.g., doxorubicin (e.g., liposomal doxorubicin)), a vinca alkaloid (e.g., vincristine) and prednisone; an anthracycline (e.g., doxorubicin (e.g., liposomal doxorubicin)), a vinca alkaloid (e.g., vincristine), prednisone and rituximab; an anthracycline (e.g., doxorubicin (e.g., liposomal doxorubicin)), a vinca alkaloid (e.g., vincristine) and demethasone; and an anthracycline (e.g., doxorubicin (e.g., liposomal doxorubicin)), a vinca alkaloid (e.g., vincristine), demethasone and rituximab.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with an alkylating agent (e.g., cyclophosphamide, dacarbazine, melphalan, ifosfamide, temozolomide) and a vinca alkaloid (e.g., vinblastine, vincristine, vindesine and vinorelbine). For example, in one embodiment, the CDP-proteasome inhibitor (such as a boronic acid containing proteasome inhibitor) conjugate, particle (e.g., nanoparticle) or composition is a CDP-bortezomib conjugate, particle (e.g., nanoparticle) or composition and the CDP-bortezomib conjugate, particle (e.g., nanoparticle) or composition is further administered in combination with an alkylating agent (e.g., cyclophosphamide) and a vinca alkaloid (e.g., vincristine). In one embodiment, the CDP-proteasome inhibitor (such as a boronic acid containing proteasome inhibitor) conjugate, particle (e.g., nanoparticle) or composition, e.g., a CDP-bortezomib conjugate, particle (e.g., nanoparticle), or composition, is further administered with one or more of prednisone, demethasone and rituximab. For example, in one embodiment, the CDP-proteasome inhibitor (such as a boronic acid containing proteasome inhibitor) conjugate, particle (e.g., nanoparticle) or composition, e.g., a CDP-bortezomib conjugate, particle (e.g., nanoparticle) or composition, is administered in one of the following combinations: an alkylating agent (e.g., cyclophosphamide), a vinca alkaloid (e.g., vincristine) and prednisone; an alkylating agent (e.g., cyclophosphamide), a vinca alkaloid (e.g., vincristine), prednisone and rituximab; an alkylating agent (e.g., cyclophosphamide), a vinca alkaloid (e.g., vincristine) and demethasone; and an alkylating agent (e.g., cyclophosphamide), a vinca alkaloid (e.g., vincristine), demethasone and rituximab.

In one embodiment, the CDP-proteasome inhibitor (such as a boronic acid containing proteasome inhibitor) conjugate, particle (e.g., nanoparticle) or composition, e.g., a CDP-bortezomib conjugate, particle (e.g., nanoparticle), or composition, is administered in combination with an anthracycline (e.g., daunorubicin, doxorubicin (e.g., liposomal doxorubicin), epirubicin, valrubicin and idarubicin) and an alkylating agent (e.g., cyclophosphamide, dacarbazine, melphalan, ifosfamide, temozolomide). For example, in one embodiment, the CDP-proteasome inhibitor (such as a boronic acid containing proteasome inhibitor) conjugate, particle (e.g., nanoparticle) or composition is a CDP-bortezomib conjugate, particle (e.g., nanoparticle) or composition and the CDP-bortezomib conjugate, particle (e.g., nanoparticle) or composition is further administered in combination with an anthracycline (e.g., doxorubicin (e.g., liposomal doxorubicin)) and an alkylating agent (e.g., cyclophosphamide). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is further administered with one or more of prednisone, demethasone and rituximab. For example, in one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in one of the following combinations: an alkylating agent (e.g., cyclophosphamide), an anthracycline (e.g., doxorubicin (e.g., liposomal doxorubicin)) and prednisone; an alkylating agent (e.g., cyclophosphamide), an anthracycline (e.g., doxorubicin (e.g., liposomal doxorubicin)), prednisone and rituximab; an alkylating agent (e.g., cyclophosphamide), an anthracycline (e.g., doxorubicin (e.g., liposomal doxorubicin)) and demethasone; an alkylating agent (e.g., cyclophosphamide), an anthracycline (e.g., doxorubicin (e.g., liposomal doxorubicin)), demethasone and rituximab.

In one embodiment, a topoisomerase inhibitor (e.g., etoposide, topotecan, irinotecan, tenoposide, SN-38, lamellarin D) can be further administered with any of the combinations described above. For example, in one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in one of the following combinations: an alkylating agent (e.g., cyclophosphamide), an anthracycline (e.g., doxorubicin (e.g., liposomal doxorubicin)), a vinca alkaloid (e.g., vincristine) and prednisone; an alkylating agent (e.g., cyclophosphamide), an anthracycline (e.g., doxorubicin (e.g., liposomal doxorubicin)), a vinca alkaloid (e.g., vincristine), prednisone and rituximab.

In one embodiment, the method further includes administering an additional chemotherapeutic treatment, wherein the additional chemotherapeutic treatment includes a combination of rituximab, an immunosuppressive agent (e.g., methotrexate) and cytarabine.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with cladribine.

In some embodiments, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with bevacizumab.

In some embodiments, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with an mTOR inhibitor. Non-limiting examples of mTOR inhibitors include rapamycin, everolimus, AP23573, CCI-779 and SDZ-RAD.

In one embodiment, the CDP-proteasome inhibitor (such as a boronic acid containing proteasome inhibitor) conjugate, particle (e.g., nanoparticle) or composition is a CDP-bortezomib conjugate, particle (e.g., nanoparticle) or composition, e.g., a CDP-bortezomib conjugate, particle (e.g., nanoparticle) or composition described herein, e.g., a CDP-bortezomib conjugate comprising bortezomib, coupled, e.g., via linkers, to a CDP described herein, particle (e.g., nanoparticle) or composition thereof. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one aspect, the disclosure features a method of treating mantle cell lymphoma, in a subject, e.g., a human. The method comprises:

providing a subject who has mantle cell lymphoma and has been treated with a chemotherapeutic agent which did not effectively treat the lymphoma (e.g., the subject has a chemotherapeutic refractory, a chemotherapeutic resistant and/or a relapsed lymphoma) or which had an unacceptable side effect (e.g., the subject has a chemotherapeutic sensitive lymphoma), and
administering a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, e.g., a CDP-bortezomib conjugate, particle (e.g., nanoparticle) or composition described herein, to a subject in an amount effective to treat the cancer, to thereby treat the cancer.

In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled, e.g., via linkers, to a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the lymphoma is refractory to, resistant to, and/or relapsed with treatment with one or more of: an alkylating agent (e.g., cyclophosphamide, dacarbazine, melphalan, ifosfamide, temozolomide), a vinca alkaloid (e.g., vinblastine, vincristine, vindesine and vinorelbine) and an anthracycline (e.g., daunorubicin, doxorubicin, epirubicin, valrubicin and idarubicin).

In one embodiment, the cancer is a multidrug resistant lymphoma.

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition can be administered in combination with one or more of: bendamustine, cladribine, fludarabine, thalidomide, a thalidomide derivative (e.g., lenalidomide), pentostatin and an mTOR inhibitor (e.g., temsirolimus). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, can further be administered in combination with an alkylating agent (e.g., cyclophosphamide, dacarbazine, melphalan, ifosfamide, temozolomide). For example, in one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in one of the following combinations: fludarabine and an alkylating agent (e.g., cyclophosphamide); fludarabine, an alkylating agent (e.g., cyclophosphamide) and mitoxantrone; fludarabine and mitoxantrone; and pentostatin and an alkylating agent (e.g., cyclophosphamide).

In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with topoisomerase inhibitor (e.g., topotecan, irinotecan, etoposide, teniposide, SN-38, lamellarin D, camptothecin (e.g., IT-101)) and an alkylating agent (e.g., cyclophosphamide, dacarbazine, melphalan, ifosfamide, temozolomide). In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is further administered in combination with prednisone and/or procarbazine.

In one embodiment, the CDP-proteasome inhibitor (such as a boronic acid containing proteasome inhibitor) conjugate, particle (e.g., nanoparticle) or composition is a CDP-bortezomib conjugate, particle (e.g., nanoparticle) or composition, e.g., a CDP-bortezomib conjugate, particle (e.g., nanoparticle) or composition described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate), forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of identifying a subject, e.g., a human, having a proliferative disorder, e.g., cancer, for treatment with a CDP-proteasome inhibitor (such as a boronic acid containing proteasome inhibitor) conjugate, particle (e.g., nanoparticle) or composition, such as a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein the method comprising

identifying a subject having a proliferative disorder who has received an anticancer agent (e.g., a boronic acid containing proteasome inhibitor such as bortezomib) and has a neutrophil count less than a standard; and identifying the subject as suitable for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein.

In one embodiment, the method further comprising administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, in an amount effective to treat the disorder.

In an embodiment, the CDP-proteasome inhibitor (such as a boronic acid containing proteasome inhibitor) conjugate comprises proteasome inhibitor molecules (such as a boronic acid containing proteasome inhibitor molecules, e.g., bortezomib molecules), coupled, e.g., via linkers, to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the standard is a neutrophil count below or equal to 1.5×10⁹ cells/L or below or equal to 0.75×10⁹ cells/L. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate), forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, with a proliferative disorder, e.g., cancer (such as a solid tumor, a liquid tumor or a semi-solid tumor), the method comprising

selecting a subject having a proliferative disease who has received an anticancer agent (e.g., a bortezomib) and has a neutrophil count less than a standard; and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject in an amount effective to treat the proliferative disorder, to thereby treat the disorder.

In an embodiment, the CDP-proteasome inhibitor (such as a boronic acid containing proteasome inhibitor) conjugate comprises proteasome inhibitor molecules (such as a boronic acid containing proteasome inhibitor molecules, e.g., bortezomib molecules), coupled, e.g., via linkers, to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the standard is a neutrophil count below or equal to 1.5×10⁹ cells/L or below or equal to 0.75×10⁹ cells/L. In some embodiments, the standard is based on a neutrophil count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate), forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for selecting a subject, e.g., a human, with a proliferative disorder, e.g., cancer, for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, comprising:

determining whether a subject with a proliferative disorder has moderate to severe neutropenia; and
selecting a subject for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, on the basis that the subject has moderate to severe neutropenia.

In an embodiment, the CDP-proteasome inhibitor (such as a boronic acid containing proteasome inhibitor) conjugate comprises proteasome inhibitor molecules (such as a boronic acid containing proteasome inhibitor molecules, e.g., bortezomib molecules), coupled, e.g., via linkers, to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein. In one embodiment, the dosing schedule is not changed between doses. For example, when the initial dosing schedule is twice a week, an additional dose is also administered twice a week. In one embodiment, the dose does not change or is decreased for an additional dose (or doses). For example, when a dose of the CDP-bortezomib conjugate is administered in an amount such that the conjugate includes 1.3 mg/m² of bortezomib, an additional dose is administered in an amount such that the conjugate includes 1.3 mg/m² or less of bortezomib. In another embodiment, the dose does not change or is increased for an additional dose (or doses). For example, when a dose of the CDP-bortezomib conjugate is administered in an amount such that the conjugate includes 1.3 mg/m² of bortezomib, an additional dose is administered in an amount such that the conjugate includes 1.3 mg/m² or more of bortezomib.

In one embodiment, the method further comprises administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject.

In one embodiment, the subject experienced moderate to severe neutropenia from treatment with an anticancer agent (e.g., a bortezomib). In one embodiment, the subject has one or more symptom of febrile neutropenia.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the standard for moderate neutropenia is a neutrophil count of 1000 to 500 cells/mm³. In one embodiment, the standard for severe neutropenia is a neutrophil count of less than 500 cells/mm³.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate), forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for treating a subject, e.g., a human, with a proliferative disorder, e.g., cancer, comprising:

selecting a subject with a proliferative disorder, e.g., cancer, who has moderate to severe neutropenia; and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject in an amount effective to treat the disorder, to thereby treat the proliferative disorder.

In an embodiment, the CDP-proteasome inhibitor (such as a boronic acid containing proteasome inhibitor) conjugate comprises proteasome inhibitor molecules (such as a boronic acid containing proteasome inhibitor molecules, e.g., bortezomib molecules), coupled, e.g., via linkers, to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein. In one embodiment, the dosing schedule is not changed between doses. For example, the CDP-bortezomib conjugate is administered twice a week for 2, 3, 4, 5, 6, 7, 8, 9, 10, 11, 12, 13, 14, 15 or more weeks. Additional doses can also be administered.

In one embodiment, the dose does not change or is decreased for an additional dose (or doses). For example, when a dose of the CDP-bortezomib conjugate is administered in an amount such that the conjugate includes 1.3 mg/m² of bortezomib, an additional dose is administered in an amount such that the conjugate includes 1.3 mg/m² or less of bortezomib. In another embodiment, the dose does not change or is increased for an additional dose (or doses). For example, when a dose of the CDP-bortezomib conjugate is administered in an amount such as that the conjugate includes 1.3 mg/m² of bortezomib, an additional dose is administered in an amount such that the conjugate includes 1.3 mg/m² or more of bortezomib.

In one embodiment, the method further comprises administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject.

In one embodiment, the subject experienced moderate to severe neutropenia from treatment with an anticancer agent (e.g., bortezomib). In one embodiment, the subject has one or more symptom of febrile neutropenia.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the standard for moderate neutropenia is a neutrophil count of 1000 to 500 cells/mm³. In one embodiment, the standard for severe neutropenia is a neutrophil count of less than 500 cells/mm³.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate), forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of identifying a subject, e.g., a human, having a proliferative disorder, e.g., cancer, for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, the method comprising

identifying a subject having a proliferative disorder who has received an anticancer agent (e.g., bortezomib) and has a platelet count less than a standard; and

identifying the subject as suitable for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein.

In one embodiment, the method further comprising administering a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein, in an amount effective to treat the disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the standard is a platelet count below or equal to 30×10⁹ cells/L. In some embodiments, the standard is based on a platelet count prior to receiving an anticancer agent, e.g., mean platelet count decreased from the mean platelet count prior to treatment with the anticancer agent, e.g., by at least 20%, 30%, 40% or 50% after administration of the anticancer agent.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, with a proliferative disorder, e.g., cancer, the method comprising:

selecting a subject having a proliferative disease who has received an anticancer agent (e.g., bortezomib) and has a platelet count less than a standard; and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject in an amount effective to treat the proliferative disorder, to thereby treat the disorder.

In an embodiment, the CDP-proteasome inhibitor (such as a boronic acid containing proteasome inhibitor) conjugate comprises proteasome inhibitor molecules (such as a boronic acid containing proteasome inhibitor molecules, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the standard is a platelet count below or equal to 30×10⁹ cells/L. In some embodiments, the standard is based on a platelet count prior to receiving an anticancer agent, e.g., mean neutrophil count decreased from the mean neutrophil count prior to treatment with the anticancer agent, e.g., by at least 20%, 30%, 40% or 50% after administration of the anticancer agent.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human, having a proliferative disorder, e.g., cancer, for treatment with a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein, the method comprising:

determining whether a subject having a proliferative disorder, e.g., cancer, has experienced a cardiac disorder from treatment with an anticancer agent, e.g., bortezomib or has or is at risk for having a cardiac disorder to treatment with an anticancer agent (e.g., bortezomib); and
selecting the subject for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein.

In one embodiment, the method further comprising administering a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein, in an amount effective to treat the disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the cardiac disorder is acute development or exacerbation of congestive heart failure. In another embodiment, the cardiac disorder is new onset or decreased left ventricular ejection fraction. In another embodiment, the cardiac disorder is one or more heart failure events selected from acute pulmonary edema, cardiac failure, congestive cardiac failure, cardigenic shock and pulmonary edema.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, with a proliferative disorder, e.g., cancer, the method comprising:

selecting a subject having a proliferative disease who has experienced a cardiac disorder from treatment with an anticancer agent, e.g., bortezomib or has or is at risk for having a cardiac disorder to treatment with an anticancer agent (e.g., bortezomib); and
administering a CDP-proteasome inhibitor (such as a boronic acid containing proteasome inhibitor) conjugate, particle (e.g., nanoparticle) or composition, e.g., a CDP-bortezomib conjugate, particle (e.g., nanoparticle) or composition described herein, to the subject in an amount effective to treat the proliferative disorder, to thereby treat the disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the cardiac disorder is acute development or exacerbation of congestive heart failure. In another embodiment, the cardiac disorder is new onset or decreased left ventricular ejection fraction. In another embodiment, the cardiac disorder is one or more heart failure events selected from acute pulmonary edema, cardiac failure, congestive cardiac failure, cardigenic shock and pulmonary edema.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human, having a proliferative disorder, e.g., cancer, for treatment with a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein, the method comprising:

determining whether a subject having a proliferative disorder, e.g., cancer, has experienced a pulmonary disorder from treatment with an anticancer agent, e.g., bortezomib or has or is at risk for having a pulmonary disorder to treatment with an anticancer agent (e.g., bortezomib); and
selecting the subject for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein.

In one embodiment, the method further comprising administering a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein, in an amount effective to treat the disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the pulmonary disorder is acute infiltrative pulmonary disease. In another embodiment, the pulmonary disorder is selected from pneumonitis, interstitial pneumonia, lung infiltration and acute respiratory distress syndrome (ARDS).

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, with a proliferative disorder, e.g., cancer, the method comprising:

selecting a subject having a proliferative disease who has experienced a pulmonary disorder from treatment with an anticancer agent, e.g., bortezomib or has or is at risk for having a pulmonary disorder to treatment with an anticancer agent (e.g., bortezomib); and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition described herein, to the subject in an amount effective to treat the proliferative disorder, to thereby treat the disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the pulmonary disorder is acute infiltrative pulmomary disease. In another embodiment, the pulmonary disorder is selected from pneumonitis, interstitial pneumonia, lung infiltration and acute respiratory distress syndrome (ARDS).

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human, having a proliferative disorder, e.g., cancer, for treatment with a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein, the method comprising:

determining whether a subject having a proliferative disorder, e.g., cancer, has experienced reversible posterior leukoencephalopath syndrome (RPLS) from treatment with an anticancer agent, e.g., bortezomib or has or is at risk for having RPLS to treatment with an anticancer agent (e.g., bortezomib); and
selecting the subject for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein.

In one embodiment, the method further comprising administering a CDP-proteasome inhibitor (such as a boronic acid containing proteasome inhibitor) conjugate, particle (e.g., nanoparticle) or composition, e.g., a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein, in an amount effective to treat the disorder.

In an embodiment, the CDP-proteasome inhibitor (such as a boronic acid containing proteasome inhibitor) conjugate comprises proteasome inhibitor molecules (such as a boronic acid containing proteasome inhibitor molecules, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the subject has experienced one or more symptoms of RPLS to treatment with an anticancer agent, e.g., bortezomib. Symptoms of RPLS includes seizure, hypertension, headache, lethargy, confusion, blindness and other visual and neurological disturbances. Brain imaging, such as MRI (magnetic resonance imaging) can be used to confirm the diagnosis.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, with a proliferative disorder, e.g., cancer, the method comprising:

selecting a subject having a proliferative disease who has experienced a pulmonary disorder from treatment with an anticancer agent, e.g., bortezomib or has or is at risk for having RPLS to treatment with an anticancer agent (e.g., bortezomib); and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject in an amount effective to treat the proliferative disorder, to thereby treat the disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the subject has experienced one or more symptoms of RPLS to treatment with an anticancer agent, e.g., bortezomib. Symptoms of RPLS includes seizure, hypertension, headache, lethargy, confusion, blindness and other visual and neurological disturbances. Brain imaging, such as MRI (magnetic resonance imaging) can be used to confirm the diagnosis.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of selecting a subject, e.g., a human with a proliferative disorder, e.g., cancer, for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, comprising:

determining whether a subject having a proliferative disorder have experienced gastrointestinal adverse events for treatment with an anticancer agent, e.g., bortezomib, or has or is at risk of having gastrointestinal adverse events to treatment with an anticancer agent, e.g., bortezomib; and
selecting the subject with a proliferative disorder, e.g., cancer, for treatment with a CDP-proteasome inhibitor (such as a boronic acid containing proteasome inhibitor) conjugate, particle (e.g., nanoparticle) or composition described herein, e.g., a CDP-bortezomib conjugate, particle (e.g., nanoparticle) or composition described herein.

In an embodiment, the CDP-proteasome inhibitor (such as a boronic acid containing proteasome inhibitor) conjugate comprises proteasome inhibitor molecules (such as a boronic acid containing proteasome inhibitor molecules, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In one embodiment, the subject has experienced one or more gastrointestinal adverse events to treatment with an anticancer agent, e.g., bortezomib. Gastrointestinal adverse events includes nausea, diarrhea, constipation and vomiting.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method of treating a subject, e.g., a human, with a proliferative disorder, e.g., cancer, the method comprising:

selecting a subject having a proliferative disease who has experienced gastrointestinal adverse events for treatment with an anticancer agent, e.g., bortezomib, or has or is at risk of having gastrointestinal adverse events to treatment with an anticancer agent, e.g., bortezomib; and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition described herein, to the subject in an amount effective to treat the proliferative disorder, to thereby treat the disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, is administered in combination with one or more additional chemotherapeutic agent, e.g., a chemotherapeutic agent or combination of chemotherapeutic agents described herein.

In one embodiment, the subject has experienced one or more gastrointestinal adverse events to treatment with an anticancer agent, e.g., bortezomib. Gastrointestinal adverse events includes nausea, diarrhea, constipation and vomiting.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for selecting a subject, e.g., a human, with a proliferative disorder, e.g., cancer, for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, comprising:

determining whether a subject with a proliferative disorder, e.g., cancer, has experienced neuropathy from treatment with an anticancer agent, e.g., bortezomib, a taxane, a vinca alkaloid, an alkylating agent, a platinum-based agent or an epothilone; and
selecting a subject for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, on the basis that the subject has experienced neuropathy from treatment with a chemotherapeutic agent, e.g., bortezomib, a taxane, a vinca alkaloid, an alkylating agent, a platinum-based agent or an epothilone.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein. In one embodiment, the dosing schedule is not changed between doses. For example, when the initial dosing schedule is twice a week, an additional dose is also administered twice a week. In one embodiment, the dose does not change or is decreased for an additional dose (or doses). For example, when a dose of the CDP-bortezomib inhibitor conjugate is administered in an amount such that the conjugate includes 1.3 mg/m² of bortezomib, an additional dose is administered in an amount such that the conjugate includes 1.3 mg/m² or less of bortezomib. In another embodiment, the dose does not change or is increased for an additional dose (or doses). For example, when a dose of the CDP-bortezomib conjugate is administered in an amount such that the conjugate includes 1.3 mg/m² of bortezomib, an additional dose is administered in an amount such that the conjugate includes 1.3 mg/m² or more of bortezomib.

In one embodiment, the neuropathy is peripheral neuropathy. In one embodiment, the neuropathy is sensory neuropathy, motor neuropathy or both.

In one embodiment, the cancer is a cancer described herein, e.g., a solid tumor, a liquid tumor or a semi-solid tumor.

In one embodiment, the cancer is multiple myeloma or mantle cell lymphoma. In one embodiment, the subject is selected for treatment with the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, in combination with one or more additional chemotherapeutic agent, e.g., a chemotherapeutic agent or combination of chemotherapeutic agents described herein.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method for treating a subject, e.g., a human, with a proliferative disorder, e.g., cancer, comprising:

selecting a subject with a proliferative disorder, e.g., cancer, who has experienced one or more symptom of neuropathy from treatment with a chemotherapeutic agent, e.g., a proteasome inhibitor (such as a boronic acid containing proteasome inhibitor, e.g. bortezomib), a taxane, a vinca alkaloid, an alkylating agent, a platinum-based agent or an epothilone; and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject in an amount effective to treat the disorder, to thereby treat the proliferative disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-bortezomib conjugate is a CDP-bortezomib conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein. In one embodiment, the dosing schedule is not changed between doses. For example, when the initial dosing schedule is twice a week, additional doses can also be administered twice a week. In one embodiment, the dose does not change or is decreased for an additional dose (or doses). For example, when a dose of the CDP-bortezomib conjugate is administered in an amount such that the conjugate includes 1.3 mg/m² of bortezomib, an additional dose is administered in an amount such that the conjugate includes 1.3 mg/m² or less of bortezomib. In another embodiment, the dose does not change or is increased for an additional dose (or doses). For example, when a dose of the CDP-bortezomib conjugate is administered in an amount such that the conjugate includes 1.3 mg/m² of bortezomib, an additional dose is administered in an amount such that the conjugate includes 1.3 mg/m² or more of bortezomib.

In one embodiment, the subject experienced moderate to severe neuropathy from treatment with a chemotherapeutic agent. In one embodiment, the neuropathy is peripheral neuropathy. In one embodiment, the neuropathy is sensory neuropathy, motor neuropathy or both.

In one embodiment, the subject has experienced neuropathy after two, three fours, five cycles of treatment with an anticancer agent.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor conjugate, particle (e.g., nanoparticle) or composition, e.g., CDP-bortezomib conjugate, particle (e.g., nanoparticle) or composition, is administered in combination with one or more additional chemotherapeutic agent, e.g., a chemotherapeutic agent or combination of chemotherapeutic agents described herein.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features a method for selecting a subject, e.g., a human, with a proliferative disorder, e.g., cancer, for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition described herein, comprising:

determining whether a subject with a proliferative disorder, e.g., cancer, has experienced a hypotension or has or is at risk for having hypotension to treatment with an anticancer agent (e.g., bortezomib), and
selecting a subject for treatment with a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, on the basis that the subject is has experienced hypotension associated with or caused by the treatment with an anticancer agent (e.g., bortezomib) or the subject has or is at risk for having hypotension to treatment with an anticancer agent (e.g., bortezomib).

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-proteasome inhibitor conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the subject has exhibited one or more symptom of hypotension to a previous treatment with the anticancer agent (e.g., bortezomib). Symptoms of hypotension include low blood pressure, lightheadedness, dizziness, seizure, chest pain, shortness of breath, irregular heartbeat, fever, headache, stiff neck, severe upper back pain, cough with phlegm, prolonged diarrhea or vomiting, dysphagia, dysuria, loss of consciousness and fatigue, temporary blurring or loss of vision.

In one embodiment, the hypotension is postural hypotension. In one embodiment, the hypotension is orthostatic hypotension. In another embodiment, the hypotension is hypotension NOS.

In one embodiment, the cancer is a cancer described herein, e.g., a solid tumor, a liquid tumor or a semi-solid tumor. In one embodiment, the cancer is multiple myeloma or mantle cell lymphoma. In one embodiment, the CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, is selected for administration in combination with one or more additional chemotherapeutic agent, e.g., a chemotherapeutic agent or combination of chemotherapeutic agents described herein.

In an embodiment, the CDP-proteasome inhibitor (such as a boronic acid containing proteasome inhibitor) conjugate (e.g., a CDP-bortezomib conjugate) forms a particle or nanoparticle having a conjugate number described herein. By way of example, a CDP-proteasome inhibitor (such as a boronic acid containing proteasome inhibitor) conjugate (e.g., a CDP-bortezomib conjugate) forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In yet another aspect, the invention features a method of treating a subject, e.g., a human, with a proliferative disorder, e.g., cancer, comprising:

selecting a subject with a proliferative disorder, e.g., cancer, who has experienced hypotension to treatment with an anticancer agent (e.g., bortezomib) or has or is at risk for having hypotension to an anticancer agent (e.g., bortezomib); and
administering a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, described herein, to the subject in an amount effective to treat the disorder, to thereby treat the proliferative disorder.

In an embodiment, the CDP-proteasome inhibitor (such as a boronic acid containing proteasome inhibitor) conjugate comprises proteasome inhibitor molecules (such as a boronic acid containing proteasome inhibitor, e.g., bortezomib molecules), coupled, e.g., via linkers, to a to a CDP moiety, e.g., a CDP described herein. In an embodiment, the CDP-proteasome inhibitor (such as a boronic acid containing proteasome inhibitor) conjugate comprises a proteasome inhibitor (such as a boronic acid containing proteasome inhibitor, e.g., bortezomib), coupled via a linker shown in Table 2 to a CDP moiety, e.g., a CDP described herein. In an embodiment, the CDP-proteasome inhibitor (such as a boronic acid containing proteasome inhibitor) conjugate is a CDP-bortezomib conjugate shown in Table 2.

In one embodiment, the CDP-proteasome inhibitor (such as a boronic acid containing proteasome inhibitor) conjugate is a CDP-bortezomib conjugate, e.g., a CDP-bortezomib conjugate described herein, e.g., a CDP-bortezomib conjugate comprising bortezomib, coupled, e.g., via linkers, to a CDP described herein. In an embodiment, the CDP-bortezomib conjugate comprises bortezomib, coupled via a linker shown in Table 2 to a CDP described herein. In an embodiment, the CDP-bortezomib conjugate is a CDP-bortezomib conjugate shown in Table 2.

In one embodiment, the CDP-bortezomib conjugate, particle (e.g., nanoparticle) or composition is administered at a dose and/or dosing schedule described herein.

In one embodiment, the subject has exhibited one or more symptom of hypotension to a previous treatment with the anticancer agent (e.g., bortezomib). Symptoms of hypotension include low blood pressure, lightheadedness, dizziness, seizure, chest pain, shortness of breath, irregular heartbeat, fever, headache, stiff neck, severe upper back pain, cough with phlegm, prolonged diarrhea or vomiting, dysphagia, dysuria, loss of consciousness and fatigue, temporary blurring or loss of vision.

In another aspect, the invention features, a method of evaluating a particle or a preparation of particles, wherein said particles, comprise one or a plurality of CDP-proteasome inhibitor (such as a boronic acid containing proteasome inhibitor) conjugate (e.g., a CDP-bortezomib conjugate) molecules. The method comprises:

providing a sample comprising one or a plurality of said particles;
determining a value for the number of CDP-proteasome inhibitor (such as a boronic acid containing proteasome inhibitor) conjugate (e.g., a CDP-bortezomib conjugate) molecules in a particle in said sample (the conjugate number),
thereby evaluating a preparation of particles.

In an embodiment the method comprises one or both of:

a) comparing said determined value with a reference value, e.g., a range of values, or
b) responsive to said determination, classifying said particles.

In an embodiment the particle is a nanoparticle.

In an embodiment the method further comprises comparing said determined value with a reference standard. In an embodiment the reference value can be selected from a value, e.g., a range, provided herein, e.g., 1 or 2 to 8, 1 or 2 to 7, 1 or 2 to 6, 1 or 2 to 5, or 2-4.

In an embodiment the reference value can be selected from a value, e.g., a range, provided herein, e.g., 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or to 75.

In an embodiment, responsive to said comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment said CDP-proteasome inhibitor (such as a boronic acid containing proteasome inhibitor) conjugate (e.g., a CDP-bortezomib conjugate) is selected from those disclosed in herein.

In an embodiment said proteasome inhibitor (such as a boronic acid containing proteasome inhibitor, e.g., bortezomib) is selected from those disclosed herein.

In an embodiment said particle is selected from those disclosed in herein.

In an embodiment, the determined value for conjugate number is compared with a reference, and responsive to said comparison said particle or preparation of particles is classified, e.g., as suitable for use in human subjects, not suitable for use in human subjects, suitable for sale, meeting a release specification, or not meeting a release specification.

In another aspect, the invention features, a particle, e.g., a nanoparticle, comprising one or more CDP-proteasome inhibitor (such as a boronic acid containing proteasome inhibitor) conjugate (e.g., a CDP-bortezomib conjugate) described herein, having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

The details of one or more embodiments of the invention are set forth in the description below. Other features, objects, and advantages of the invention will be apparent from the description and the drawings, and from the claims.

### BRIEF DESCRIPTION OF THE FIGURES

FIG. 1 depicts an exemplary synthetic scheme (Scheme I) for covalently bonding a derivatized CD to a boronic acid, wherein the boronic acid is complexed with any of the 1,2-diols situated on the rim of the CD (with R representing the remainder of the boronic acid).

FIG. 2 depicts a general strategy (Scheme II) for synthesizing linear, branched or grafted cyclodextrin-containing polymers (CDPs) for loading a boronic acid, and an optional targeting ligand.

FIG. 3 depicts a general scheme for graft polymers (Scheme Ha).

FIG. 4 depicts a general scheme of preparing linear CDPs (Scheme IIb).

FIG. 5 depicts the dependence of a 66.5 kDa conjugate particle size on conjugate number.

### DETAILED DESCRIPTION OF THE INVENTION

The present invention relates to novel compositions of therapeutic cyclodextrin-containing polymers conjugated to a proteasome inhibitor (such as a boronic acid containing proteasome inhibitor, e.g., bortezomib), and methods of use thereof. In certain embodiments, these cyclodextrin-containing polymers improve stability and/or solubility, and/or reduce toxicity, and/or improve efficacy of the proteasome inhibitor (such as a boronic acid containing proteasome inhibitor, e.g., bortezomib) when used in vivo.

By selecting from a variety of linker groups used to link a proteasome inhibitor (such as a boronic acid containing proteasome inhibitor, e.g., bortezomib) to a CDP, the rate of release of proteasome inhibitor (such as a boronic acid containing proteasome inhibitor, e.g., bortezomib) from the CDP can be attenuated for controlled delivery. The invention also relates to methods of treating subjects, e.g., humans, with a CDP-proteasome inhibitor (such as a boronic acid containing proteasome inhibitor) conjugate, e.g., a CDP-bortezomib conjugate described herein. The invention further relates to methods for conducting a pharmaceutical business comprising manufacturing, licensing, or distributing kits containing or relating to the CDP-proteasome inhibitor (such as a boronic acid containing proteasome inhibitor) conjugate, e.g., a CDP-bortezomib conjugate, described herein.

More generally, the present invention provides water-soluble, biocompatible polymer conjugates comprising a water-soluble, biocompatible cyclodextrin containing polymer covalently attached to a proteasome inhibitor (such as a boronic acid containing proteasome inhibitor, e.g., bortezomib) through attachments that are cleaved under biological conditions to release the proteasome inhibitor (such as a boronic acid containing proteasome inhibitor, e.g., bortezomib).

Polymeric conjugates featured in the present invention may be useful to improve solubility and/or stability of a bioactive/therapeutic agent, such as a proteasome inhibitor (such as a boronic acid containing proteasome inhibitor, e.g., bortezomib), reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues. Poorly soluble and/or toxic compounds may benefit particularly from incorporation into polymeric compounds of the invention.

An “effective amount” or “an amount effective” refers to an amount of the CDP-proteasome inhibitor (such as a boronic acid containing proteasome inhibitor, e.g., bortezomib) conjugate which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition is outweighed by the therapeutically beneficial effects. Unless specified otherwise, the CDP-proteasome inhibitor (such as a boronic acid containing proteasome inhibitor) conjugate, e.g., a CDP-bortezomib conjugate, and other pharmaceutically active agent described herein are administered in an effective amount.

“Pharmaceutically acceptable carrier or adjuvant,” as used herein, refers to a carrier or adjuvant that may be administered to a patient, together with a CDP-proteasome inhibitor (such as a boronic acid containing proteasome inhibitor) conjugate, e.g., a CDP-bortezomib conjugate, described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

In one embodiment, the composition of the present invention comprises a CDP-proteasome inhibitor (such as a boronic acid containing proteasome inhibitor) conjugate or particle (e.g., nanoparticle), e.g., CDP-bortezomib conjugate or particle (e.g., nanoparticle) described herein, and mannitol.

As used herein the term “low aqueous solubility” refers to water insoluble compounds having poor solubility in water, that is <5 mg/ml at physiological pH (6.5-7.4). Preferably, their water solubility is <1 mg/ml, more preferably <0.1 mg/ml. It is desirable that the drug is stable in water as a dispersion; otherwise a lyophilized or spray-dried solid form may be desirable.

As used herein, the term “prevent” or “preventing” as used in the context of the administration of a conjugate, particle (e.g., nanoparticle), or composition to a subject, refers to subjecting the subject to a regimen, e.g., the administration of a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, such that the onset of at least one symptom of the disorder is delayed as compared to what would be seen in the absence of the regimen.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a CDP-proteasome inhibitor (such as a boronic acid containing proteasome inhibitor) conjugate, particle (e.g., nanoparticle) or composition, e.g., CDP-bortezomib conjugate, particle (e.g., nanoparticle) or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “alkenyl” refers to an aliphatic group containing at least one double bond.

The terms “alkoxyl” or “alkoxy” refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure.

The term “alkynyl” refers to an aliphatic group containing at least one triple bond.

The term “aralkyl” or “arylalkyl” refers to an alkyl group substituted with an aryl group (e.g., a phenyl or naphthyl).

The term “aryl” includes 5-14 membered single-ring or bicyclic aromatic groups, for example, benzene, naphthalene, and the like. The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. Each ring can contain, e.g., 5-7 members. The term “arylene” refers to a divalent aryl, as defined herein.

The term “arylalkenyl” refers to an alkenyl group substituted with an aryl group.

The terms “halo” and “halogen” means halogen and includes chloro, fluoro, bromo, and iodo.

The terms “hetaralkyl”, “heteroaralkyl” or “heteroarylalkyl” refers to an alkyl group substituted with a heteroaryl group.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylene” refers to a divalent heteroaryl, as defined herein.

The term “heteroarylalkenyl” refers to an alkenyl group substituted with a heteroaryl group.

### CDP-Proteasome Inhibitor Conjugates

Described herein are cyclodextrin containing polymer (“CDP”)-proteasome inhibitor (such as a boronic acid containing proteasome inhibitor) conjugates, e.g., CDP-bortezomib conjugates, wherein one or more proteasome inhibitors (such as a boronic acid containing proteasome inhibitor, e.g., bortezomib) are covalently attached to the CDP (e.g., either directly or through a linker). The CDP-proteasome inhibitor (such as a boronic acid containing proteasome inhibitor) conjugates, e.g., a CDP-bortezomib conjugates, include linear or branched cyclodextrin-containing polymers and polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952.

Accordingly, in one embodiment the CDP-proteasome inhibitor (such as a boronic acid containing proteasome inhibitor) conjugate, e.g., a CDP-bortezomib conjugate, is represented by Formula (1):

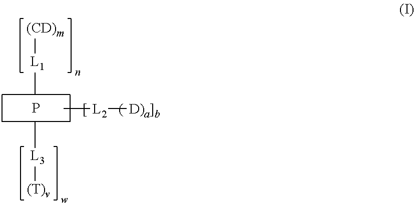

wherein

P represents a linear or branched polymer chain;

CD represents a cyclic moiety such as a cyclodextrin moiety;

L₁, L₂ and L₃, independently for each occurrence, may be absent or represent a linker group;

D, independently for each occurrence, represents a proteasome inhibitor (such as a boronic acid containing proteasome inhibitor, e.g., bortezomib) or a prodrug thereof;

T, independently for each occurrence, represents a targeting ligand or precursor thereof;

a, m, and v, independently for each occurrence, represent integers in the range of 1 to 10 (preferably 1 to 8, 1 to 5, or even 1 to 3);

n and w, independently for each occurrence, represent an integer in the range of 0 to about 30,000 (preferably <25,000, <20,000, <15,000, <10,000, <5,000, <1,000, <500, <100, <50, <25, <10, or even <5); and

b represents an integer in the range of 1 to about 30,000 (preferably <25,000, <20,000, <15,000, <10,000, <5,000, <1,000, <500, <100, <50, <25, <10, or even <5),

wherein either P comprises cyclodextrin moieties or n is at least 1.

In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate, e.g., a CDP-bortezomib conjugate, can be replaced with another therapeutic agent, e.g., another anticancer agent or anti-inflammatory agent. Examples of other anticancer agents are described herein. Examples of anti-inflammatory agents include a steroid, e.g., prednisone, and a NSAID.

In certain embodiments, P contains a plurality of cyclodextrin moieties within the polymer chain as opposed to the cyclodextrin moieties being grafted onto pendant groups off of the polymeric chain. Thus in certain embodiments, the polymer chain of formula I further comprises n′ units of U′, wherein n′ represents an integer in the range of 1 to about 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5); and U′ is represented by one of the general formulae below:

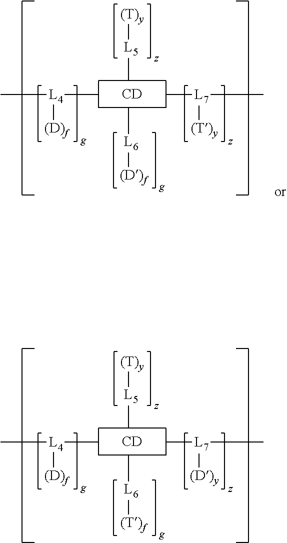

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different proteasome inhibitor (such as a boronic acid containing proteasome inhibitor, e.g., bortezomib) or prodrug forms thereof;

T and T′, independently for each occurrence, represent the same or different targeting ligand or precursor thereof;

f and y, independently for each occurrence, represent an integer in the range of 1 and 10; and

g and z, independently for each occurrence, represent an integer in the range of 0 and 10.

Preferably the polymer has a plurality of D or D′ moieties. In some embodiments, at least 50% of the U′ units have at least one D or D′. In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate, e.g., a CDP-bortezomib conjugate, can be replaced with another therapeutic agent, e.g., another anticancer agent or anti-inflammatory agent.

In preferred embodiments, L₄ and L₇ represent linker groups.

The CDP may include a polycation, polyanion, or non-ionic polymer. A polycationic or polyanionic polymer has at least one site that bears a positive or negative charge, respectively. In certain such embodiments, at least one of the linker moiety and the cyclic moiety comprises such a charged site, so that every occurrence of that moiety includes a charged site. In some embodiments, the CDP is biocompatible.

In certain embodiments, the CDP may include polysaccharides, and other non-protein biocompatible polymers, and combinations thereof, that contain at least one terminal hydroxyl group, such as polyvinylpyrrollidone, poly(oxyethylene)glycol (PEG), polysuccinic anhydride, polysebacic acid, PEG-phosphate, polyglutamate, polyethylenimine, maleic anhydride divinylether (DIVMA), cellulose, pullulans, inulin, polyvinyl alcohol (PVA), N-(2-hydroxypropyl)methacrylamide (HPMA), dextran and hydroxyethyl starch (HES), and have optional pendant groups for grafting therapeutic agents, targeting ligands and/or cyclodextrin moieties. In certain embodiments, the polymer may be biodegradable such as poly(lactic acid), poly(glycolic acid), poly(alkyl 2-cyanoacrylates), polyanhydrides, and polyorthoesters, or bioerodible such as polylactide-glycolide copolymers, and derivatives thereof, non-peptide polyaminoacids, polyiminocarbonates, poly alpha-amino acids, polyalkyl-cyano-acrylate, polyphosphazenes or acyloxymethyl poly aspartate and polyglutamate copolymers and mixtures thereof.

In another embodiment the CDP-proteasome inhibitor (such as a boronic acid containing proteasome inhibitor) conjugate, e.g., a CDP-bortezomib conjugate, is represented by Formula (II):

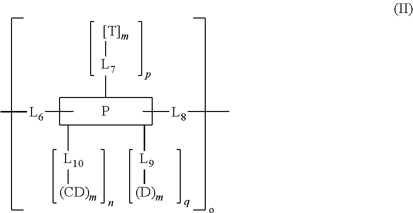

wherein

P represents a monomer unit of a polymer that comprises cyclodextrin moieties;

T, independently for each occurrence, represents a targeting ligand or a precursor thereof;

L₆, L₇, L₈, L₉, and L₁₀, independently for each occurrence, may be absent or represent a linker group;

CD, independently for each occurrence, represents a cyclodextrin moiety or a derivative thereof;

D, independently for each occurrence, represents a proteasome inhibitor (such as a boronic acid containing proteasome inhibitor, e.g., bortezomib) or a prodrug form thereof;

m, independently for each occurrence, represents an integer in the range of 1 to 10 (preferably 1 to 8, 1 to 5, or even 1 to 3);

o represents an integer in the range of 1 to about 30,000 (preferably <25,000, <20,000, <15,000, <10,000, <5,000, <1,000, <500, <100, <50, <25, <10, or even <5); and

p, n, and q, independently for each occurrence, represent an integer in the range of 0 to 10 (preferably 0 to 8, 0 to 5, 0 to 3, or even 0 to about 2),

wherein CD and D are preferably each present at least 1 location (preferably at least 5, 10, 25, or even 50 or 100 locations) in the compound.

In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate, e.g., a CDP-bortezomib conjugate, can be replaced with another therapeutic agent, e.g., another anticancer agent or anti-inflammatory agent. Examples of an anticancer agent are described herein. Examples of anti-inflammatory agents include a steroid, e.g., prednisone, or a NSAID.

In another embodiment the CDP-proteasome inhibitor (such as a boronic acid containing proteasome inhibitor) conjugate, e.g., a CDP-bortezomib conjugate, is represented formula (III) or (IV) below:

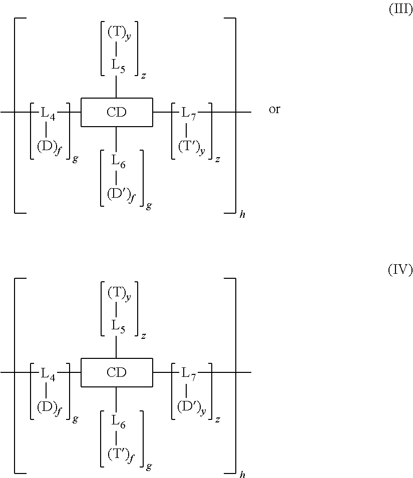

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different proteasome inhibitor (such as a boronic acid containing proteasome inhibitor, e.g., bortezomib) or prodrug thereof;

T and T′, independently for each occurrence, represent the same or different targeting ligand or precursor thereof;

f and y, independently for each occurrence, represent an integer in the range of 1 and 10 (preferably 1 to 8, 1 to 5, or even 1 to 3);

g and z, independently for each occurrence, represent an integer in the range of 0 and 10 (preferably 0 to 8, 0 to 5, 0 to 3, or even 0 to about 2); and

h represents an integer in the range of 1 and 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5),

wherein at least one occurrence (and preferably at least 5, 10, or even at least 20, 50, or 100 occurrences) of g represents an integer greater than 0.

Preferably the polymer has a plurality of D or D′ moieties. In some embodiments, at least 50% of the polymer repeating units have at least one D or D′. In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate, e.g., a CDP-bortezomib conjugate, can be replaced with another therapeutic agent, e.g., another anticancer agent or anti-inflammatory agent.

In preferred embodiments, L₄ and L₇ represent linker groups.

In certain such embodiments, the CDP comprises cyclic moieties alternating with linker moieties that connect the cyclic structures, e.g., into linear or branched polymers, preferably linear polymers. The cyclic moieties may be any suitable cyclic structures, such as cyclodextrins, crown ethers (e.g., 18-crown-6,15-crown-5,12-crown-4, etc.), cyclic oligopeptides (e.g., comprising from 5 to 10 amino acid residues), cryptands or cryptates (e.g., cryptand [2.2.2], cryptand-2,1,1, and complexes thereof), calixarenes, or cavitands, or any combination thereof. Preferably, the cyclic structure is (or is modified to be) water-soluble. In certain embodiments, e.g., for the preparation of a linear polymer, the cyclic structure is selected such that under polymerization conditions, exactly two moieties of each cyclic structure are reactive with the linker moieties, such that the resulting polymer comprises (or consists essentially of) an alternating series of cyclic moieties and linker moieties, such as at least four of each type of moiety. Suitable difunctionalized cyclic moieties include many that are commercially available and/or amenable to preparation using published protocols. In certain embodiments, conjugates are soluble in water to a concentration of at least 0.1 g/mL, preferably at least 0.25 g/mL.

Thus, in certain embodiments, the invention relates to novel compositions of therapeutic cyclodextrin-containing polymeric compounds designed for drug delivery of a proteasome inhibitor (such as a boronic acid containing proteasome inhibitor, e.g., bortezomib). In certain embodiments, these CDPs improve drug stability and/or solubility, and/or reduce toxicity, and/or improve efficacy of the proteasome inhibitor (such as a boronic acid containing proteasome inhibitor, e.g., bortezomib) when used in vivo. Furthermore, by selecting from a variety of linker groups, and/or targeting ligands, the rate of release for the proteasome inhibitor(s) (such as a boronic acid containing proteasome inhibitor, e.g., bortezomib) from the CDP can be attenuated for controlled delivery.

In certain embodiments, the CDP comprises a linear cyclodextrin-containing polymer, e.g., the polymer backbone includes cyclodextrin moieties. For example, the polymer may be a water-soluble, linear cyclodextrin polymer produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety covalently linking two cyclodextrin moieties. In yet another embodiment, the polymer is a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

Described herein are CDP-proteasome inhibitor (such as a boronic acid containing proteasome inhibitor, e.g., bortezomib) conjugates, wherein one or more proteasome inhibitor (such as a boronic acid containing proteasome inhibitor, e.g., bortezomib) is covalently attached to the CDP. The CDP can include linear or branched cyclodextrin-containing polymers and/or polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952, which are incorporated herein by reference in their entirety.

In some embodiments, the CDP-proteasome inhibitor (such as a boronic acid containing proteasome inhibitor) conjugate, e.g., a CDP-bortezomib conjugate, comprises a water soluble linear polymer conjugate comprising: cyclodextrin moieties; comonomers which do not contain cyclodextrin moieties (comonomers); and a plurality of proteasome inhibitors (such as a boronic acid containing proteasome inhibitor, e.g., bortezomib);

wherein the CDP-proteasome inhibitor (such as a boronic acid containing proteasome inhibitor) conjugate, e.g., the CDP-bortezomib conjugate, comprises at least four, five, six, seven, eight, etc., cyclodextrin moieties and at least four, five, six, seven, eight, etc., comonomers. In some embodiments, the proteasome inhibitor (such as a boronic acid containing proteasome inhibitor, e.g., bortezomib) is a proteasome inhibitor (such as a boronic acid containing proteasome inhibitor, e.g., bortezomib) described herein, for example, the proteasome inhibitor (such as a boronic acid containing proteasome inhibitor, e.g., bortezomib) is a compound comprising a boronic acid or a boronic acid derivative described herein. In some embodiments, the proteasome inhibitor is bortezomib. The proteasome inhibitor (such as a boronic acid containing proteasome inhibitor, e.g., bortezomib) can be attached to the CDP via a linker group comprising a functional group such as an amino group.

In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate, e.g., CDP-bortezomib conjugate, can be replaced with another therapeutic agent, e.g., another anticancer agent or anti-inflammatory agent.

In some embodiments, the least four cyclodextrin moieties and at least four comonomers alternate in the CDP-proteasome inhibitor (such as a boronic acid containing proteasome inhibitor) conjugate, e.g., CDP-bortezomib conjugate. In some embodiments, said proteasome inhibitor (such as a boronic acid containing proteasome inhibitor, e.g., bortezomib) are cleaved from said CDP-proteasome inhibitor (such as a boronic acid containing proteasome inhibitor, e.g., bortezomib) conjugate under biological conditions to release proteasome inhibitor (such as a boronic acid containing proteasome inhibitor, e.g., bortezomib). In some embodiments, the cyclodextrin moieties comprise linkers to which the proteasome inhibitors (such as a boronic acid containing proteasome inhibitor, e.g., bortezomib) are linked. In some embodiments, the proteasome inhibitors (such as a boronic acid containing proteasome inhibitor, e.g., bortezomib) are attached via linkers.

In some embodiments, the comonomer comprises residues of at least two functional groups through which reaction and linkage of the cyclodextrin monomers was achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a proteasome inhibitor (such as a boronic acid containing proteasome inhibitor, e.g., bortezomib) was achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C1-C10 alkyl, or arylalkyl optionally containing one or more heteroatoms within the chain or ring. In some embodiments, the cyclodextrin moiety comprises an alpha, beta, or gamma cyclodextrin moiety. In some embodiments, the proteasome inhibitor (such as a boronic acid containing proteasome inhibitor, e.g., bortezomib) is at least 5%, 10%, 15%, 20%, 25%, 30%, or 35% by weight of the CDP-proteasome inhibitor (such as a boronic acid containing proteasome inhibitor) conjugate, e.g., a CDP-bortezomib conjugate.

In some embodiments, the comonomer comprises polyethylene glycol of molecular weight 3,400 Da, the cyclodextrin moiety comprises beta-cyclodextrin, the theoretical maximum loading of the proteasome inhibitor (such as a boronic acid containing proteasome inhibitor, e.g., bortezomib) on the CDP-proteasome inhibitor (such as a boronic acid containing proteasome inhibitor) conjugate, e.g., a CDP-bortezomib conjugate, is 13% by weight, and the proteasome inhibitor (such as a boronic acid containing proteasome inhibitor, e.g., bortezomib) is 6-10% by weight of the CDP-proteasome inhibitor (such as a boronic acid containing proteasome inhibitor) conjugate, e.g., a CDP-bortezomib conjugate. In some embodiments, the proteasome inhibitor (such as a boronic acid containing proteasome inhibitor, e.g., bortezomib) is poorly soluble in water. In some embodiments, the solubility of the proteasome inhibitor (such as a boronic acid containing proteasome inhibitor, e.g., bortezomib) is <5 mg/ml at physiological pH. In some embodiments, the proteasome inhibitor (such as a boronic acid containing proteasome inhibitor, e.g., bortezomib) is a hydrophobic compound with a log P>0.4, >0.6, >0.8, >1, >2, >3, >4, or >5.

In some embodiments, the proteasome inhibitor (such as a boronic acid containing proteasome inhibitor, e.g., bortezomib) is attached to the CDP via a second compound.

In some embodiments, administration of the CDP-proteasome inhibitor (such as a boronic acid containing proteasome inhibitor) conjugate, e.g., a CDP-bortezomib conjugate to a subject results in release of the proteasome inhibitor (such as a boronic acid containing proteasome inhibitor, e.g., bortezomib) over a period of at least 6 hours. In some embodiments, administration of the CDP-proteasome inhibitor (such as a boronic acid containing proteasome inhibitor) conjugate, e.g., a CDP-bortezomib conjugate, to a subject results in release of the proteasome inhibitor (such as a boronic acid containing proteasome inhibitor, e.g., bortezomib) over a period of 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month. In some embodiments, upon administration of the CDP-proteasome inhibitor (such as a boronic acid containing proteasome inhibitor) conjugate, e.g., a CDP-bortezomib conjugate, to a subject the rate of proteasome inhibitor (such as a boronic acid containing proteasome inhibitor, e.g., bortezomib) release is dependent primarily upon the rate of hydrolysis as opposed to enzymatic cleavage.

In some embodiments, the CDP-proteasome inhibitor (such as a boronic acid containing proteasome inhibitor) conjugate, e.g., a CDP-bortezomib conjugate, has a molecular weight of 10,000-500,000. In some embodiments, the cyclodextrin moieties make up at least about 2%, 5%, 10%, 20%, 30%, 50% or 80% of the CDP-proteasome inhibitor (such as a boronic acid containing proteasome inhibitor) conjugate, e.g., a CDP-bortezomib conjugate, by weight.

In some embodiments, the CDP-proteasome inhibitor (such as a boronic acid containing proteasome inhibitor) conjugate, e.g., a CDP-bortezomib conjugate, is made by a method comprising providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced. In some embodiments, the cyclodextrin moiety precursors are in a composition, the composition being substantially free of cyclodextrin moieties having other than two positions modified to bear a reactive site (e.g., cyclodextrin moieties having 1, 3, 4, 5, 6, or 7 positions modified to bear a reactive site).

In some embodiments, a comonomer of the CDP-proteasome inhibitor (such as a boronic acid containing proteasome inhibitor) conjugate, e.g., a CDP-bortezomib conjugate, comprises a moiety selected from the group consisting of: an alkylene chain, polysuccinic anhydride, poly-L-glutamic acid, poly(ethyleneimine), an oligosaccharide, and an amino acid chain. In some embodiments, a CDP-proteasome inhibitor (such as a boronic acid containing proteasome inhibitor) conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1—C(NR₁)—NR₁—, and —B(OR₁)—; and R₁, independently for each occurrence, represents H or a lower alkyl.

In some embodiments, the CDP-proteasome inhibitor (such as a boronic acid containing proteasome inhibitor) conjugate is a polymer having attached thereto a plurality of D moieties of the following formula:

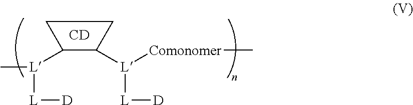

wherein each L′ is independently a first linker connecting CD and Comonomer and comprising a functional group bonded to linker L, L is a linker connecting L′ and D; and each D is independently a proteasome inhibitor (such as a boronic acid containing proteasome inhibitor, e.g., bortezomib), a prodrug derivative thereof, or absent; and each comonomer is independently a comonomer described herein, and n is at least 4, 5, 6, 7, 8, 9, 10, 11, 12, 13, 14, 15, 16, 17, 18, 19 or 20, provided that the polymer comprises at least one proteasome inhibitor (such as a boronic acid containing proteasome inhibitor, e.g., bortezomib) and in some embodiments, at least two proteasome inhibitor (such as a boronic acid containing proteasome inhibitor, e.g., bortezomib) moieties. In some embodiments, the molecular weight of the comonomer is from about 2000 to about 5000 Da (e.g., from about 3000 to about 4000 Da (e.g., about 3400 Da).

In some embodiments, the proteasome inhibitor (such as a boronic acid containing proteasome inhibitor, e.g., bortezomib) is a proteasome inhibitor described herein, for example, the proteasome inhibitor is a boronic acid containing proteasome inhibitor. As used herein, a boronic acid containing proteasome inhibitor is a proteasome inhibitor comprising a boronic acid moiety or a boronic acid derivative described herein. In some embodiments, the proteasome inhibitor is bortezomib. The proteasome inhibitor (such as a boronic acid containing proteasome inhibitor, e.g., bortezomib) can be attached to the CDP via a linker group comprising a functional group such as an amino group. In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate can be replaced with another therapeutic agent, e.g., another anticancer agent or anti-inflammatory agent.

In some embodiments, the CDP-proteasome inhibitor (such as a boronic acid containing proteasome inhibitor) conjugate is a polymer having attached thereto a plurality of D moieties of the following formula:

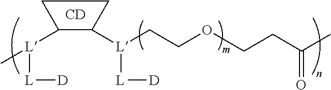

wherein each L′ is independently a first linker connecting CD and the

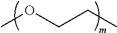

group and comprising a functional group bonded to linker L, each L is independently a linker connecting L′ and D, and each D is independently proteasome inhibitor (such as a boronic acid containing proteasome inhibitor, e.g., bortezomib), a prodrug derivative thereof, or absent, provided that the polymer comprises at least one proteasome inhibitor (such as a boronic acid containing proteasome inhibitor, e.g., bortezomib) and in some embodiments, at least two proteasome inhibitor (such as a boronic acid containing proteasome inhibitor, e.g., bortezomib) moieties; and

wherein the group

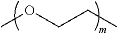

has a Mw of 3.4 kDa or less and n is at least 4, 5, 6, 7, 8, 9, 10, 11, 12, 13, 14, 15, 16, 17, 18, 19 or 20.

In some embodiments, the proteasome inhibitor is a proteasome inhibitor described herein, for example, the proteasome inhibitor is a boronic acid containing proteasome inhibitor described herein. In some embodiments, the proteasome inhibitor is bortezomib. The proteasome inhibitor (such as a boronic acid containing proteasome inhibitor, e.g., bortezomib) can be attached to the CDP via a linker group comprising a functional group such as an amino group. In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate can be replaced with another therapeutic agent, e.g., another anticancer agent or anti-inflammatory agent.

In some embodiments, less than all of the L moieties are attached to D moieties, meaning in some embodiments, at least one D is absent. In some embodiments, the loading of the D moieties on the CDP-proteasome inhibitor (such as a boronic acid containing proteasome inhibitor) conjugate is from about 1 to about 50% (e.g., from about 1 to about 25%, from about 5 to about 20% or from about 5 to about 15%). In some embodiments, each L independently comprises an amino acid or a derivative thereof. In some embodiments, each L independently comprises a plurality of amino acids or derivatives thereof. In some embodiments, each L is independently a dipeptide or derivative thereof.

In some embodiments, the CDP-proteasome inhibitor (such as a boronic acid containing proteasome inhibitor) conjugate is a polymer having attached thereto a plurality of L-D moieties of the following formula:

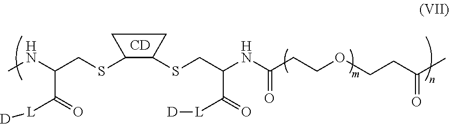

wherein each L is independently a linker or absent and each D is independently-U-proteasome inhibitor (such as a boronic acid containing proteasome inhibitor, e.g., bortezomib), a prodrug derivative thereof, or absent and wherein the group

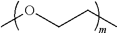

has a Mw of 3.4 kDa or less and n is at least 4, 5, 6, 7, 8, 9, 10, 11, 12, 13, 14, 15, 16, 17, 18, 19 or 20, provided that the polymer comprises at least one proteasome inhibitor (such as a boronic acid containing proteasome inhibitor, e.g., bortezomib) and in some embodiments, at least two proteasome inhibitor (such as a boronic acid containing proteasome inhibitor, e.g., bortezomib) moieties.

In some embodiments, less than all of the C(═O) moieties are attached to L-D moieties, meaning in some embodiments, at least one L and/or D is absent. In some embodiments, the loading of the L, D and/or L-D moieties on the CDP-proteasome inhibitor (such as a boronic acid containing proteasome inhibitor) conjugate, e.g., a CDP-bortezomib conjugate, is from about 1 to about 50% (e.g., from about 1 to about 25%, from about 5 to about 20% or from about 5 to about 15%). In some embodiments, each L is independently an amino acid or derivative thereof. In some embodiments, each L is glycine or a derivative thereof.

In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate, e.g., a CDP-bortezomib conjugate, can be replaced with another therapeutic agent, e.g., another anticancer agent or anti-inflammatory agent.

In some embodiments, the CDP-proteasome inhibitor (such as a boronic acid containing proteasome inhibitor) conjugate, e.g., a CDP-bortezomib conjugate, is a polymer having the following formula:

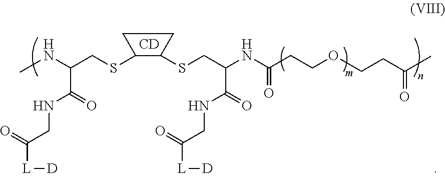

In some embodiments, less than all of the C(═O) moieties are attached to

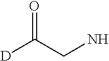

moieties, meaning in some embodiments,

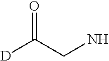

is absent, provided that the polymer comprises at least one proteasome inhibitor (such as a boronic acid containing proteasome inhibitor, e.g., bortezomib) and in some embodiments, at least two proteasome inhibitor (such as a boronic acid containing proteasome inhibitor, e.g., bortezomib) moieties. In some embodiments, the loading of the

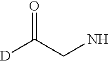

moieties on the CDP-proteasome inhibitor (such as a boronic acid containing proteasome inhibitor) conjugate is from about 1 to about 50% (e.g., from about 1 to about 25%, from about 5 to about 20% or from about 5 to about 15%).

In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor, e.g., bortezomib) conjugate can be replaced with another therapeutic agent, e.g., another anticancer agent or anti-inflammatory agent.

In some embodiments, the CDP-proteasome inhibitor (such as a boronic acid containing proteasome inhibitor) conjugate will contain a proteasome inhibitor (such as a boronic acid containing proteasome inhibitor, e.g., bortezomib) and at least one additional therapeutic agent. For instance, a proteasome inhibitor (such as a boronic acid containing proteasome inhibitor, e.g., bortezomib) and one more different cancer drugs, an immunosuppressant, an antibiotic or an anti-inflammatory agent may be grafted on to the polymer via optional linkers. By selecting different linkers for different drugs, the release of each drug may be attenuated to achieve maximal dosage and efficacy.

### Cyclodextrins

In certain embodiments, the cyclodextrin moieties make up at least about 2%, 5% or 10% by weight, up to 20%, 30%, 50% or even 80% of the CDP by weight. In certain embodiments, the proteasome inhibitor (such as a boronic acid containing proteasome inhibitor, e.g., bortezomib), or targeting ligands make up at least about 1%, 5%, 10% or 15%, 20%, 25%, 30% or even 35% of the CDP by weight. Number-average molecular weight (Mₙ) may also vary widely, but generally fall in the range of about 1,000 to about 500,000 daltons, preferably from about 5000 to about 200,000 daltons and, even more preferably, from about 10,000 to about 100,000. Most preferably, Mₙ varies between about 12,000 and 65,000 daltons. In certain other embodiments, Mₙ varies between about 3000 and 150,000 daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha ((α)-cyclodextrins, beta (β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

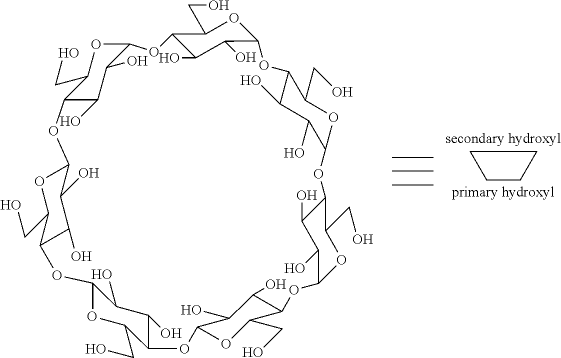

The side on which the secondary hydroxyl groups are located has a wider diameter than the side on which the primary hydroxyl groups are located. The present invention contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332 (1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

In certain embodiments, the compounds comprise cyclodextrin moieties and wherein at least one or a plurality of the cyclodextrin moieties of the CDP-proteasome inhibitor (such as a boronic acid containing proteasome inhibitor, e.g., bortezomib) conjugate is oxidized. In certain embodiments, the cyclodextrin moieties of P alternate with linker moieties in the polymer chain.

### Comonomers

In addition to a cyclodextrin moiety, the CDP can also include a comonomer, for example, a comonomer described herein. In some embodiments, a comonomer of the CDP-proteasome inhibitor (such as a boronic acid containing proteasome inhibitor) conjugate, e.g., a CDP-bortezomib conjugate, comprises a moiety selected from the group consisting of: an alkylene chain, polysuccinic anhydride, poly-L-glutamic acid, poly(ethyleneimine), an oligosaccharide, and an amino acid chain. In some embodiments, a CDP-proteasome inhibitor (such as a boronic acid containing proteasome inhibitor) conjugate, e.g., a CDP-bortezomib conjugate, comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, a comonomer can be and/or can comprise a linker such as a linker described herein.

### Linkers/Tethers

The CDPs described herein can include one or more linkers. In some embodiments, a linker, such as a linker described herein, can link a cyclodextrin moiety to a comonomer. In some embodiments, a linker can link a proteasome inhibitor (such as a boronic acid containing proteasome inhibitor, e.g., bortezomib) to a CDP. In some embodiments, for example, when referring to a linker that links a proteasome inhibitor (such as a boronic acid containing proteasome inhibitor, e.g., bortezomib) to the CDP, the linker can be referred to as a tether.

In certain embodiments, a plurality of the linker moieties are attached to a proteasome inhibitor (such as a boronic acid containing proteasome inhibitor, e.g., bortezomib) or prodrug thereof and are cleaved under biological conditions.

Described herein are CDP-proteasome inhibitor (such as a boronic acid containing proteasome inhibitor) conjugates that comprise a CDP covalently attached to proteasome inhibitor (such as a boronic acid containing proteasome inhibitor, e.g., bortezomib) through attachments that are cleaved under biological conditions to release the proteasome inhibitor (such as a boronic acid containing proteasome inhibitor, e.g., bortezomib). In certain embodiments, a CDP-proteasome inhibitor (such as a boronic acid containing proteasome inhibitor) conjugate comprises a proteasome inhibitor (such as a boronic acid containing proteasome inhibitor, e.g., bortezomib) covalently attached to a polymer, preferably a biocompatible polymer, through a tether, e.g., a linker, wherein the tether comprises a selectivity-determining moiety and a self-cyclizing moiety which are covalently attached to one another in the tether, e.g., between the polymer and the proteasome inhibitor (such as a boronic acid containing proteasome inhibitor, e.g., bortezomib).

In some embodiments, such proteasome inhibitors are covalently attached to CDPs through functional groups comprising one or more heteroatoms, for example, hydroxy, thiol, carboxy, amino, and amide groups. Such groups may be covalently attached to the subject polymers through linker groups as described herein, for example, biocleavable linker groups, and/or through tethers, such as a tether comprising a selectivity-determining moiety and a self-cyclizing moiety which are covalently attached to one another.

In certain embodiments, the CDP-proteasome inhibitor (such as a boronic acid containing proteasome inhibitor) conjugate comprises a proteasome inhibitor covalently attached to the CDP through a tether, wherein the tether comprises a self-cyclizing moiety. In some embodiments, the tether further comprises a selectivity-determining moiety. Thus, one aspect of the invention relates to a polymer conjugate comprising a therapeutic agent covalently attached to a polymer, preferably a biocompatible polymer, through a tether, wherein the tether comprises a selectivity-determining moiety and a self-cyclizing moiety which are covalently attached to one another.

In some embodiments, the selectivity-determining moiety is bonded to the self-cyclizing moiety between the self-cyclizing moiety and the CDP.

In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, the invention contemplates any combination of the foregoing. Those skilled in the art will recognize that, for example, any CDP of the invention in combination with any linker (e.g., self-cyclizing moiety, any selectivity-determining moiety, and/or any proteasome inhibitor) are within the scope of the invention.

In certain embodiments, the selectivity-determining moiety is selected such that the bond is cleaved under acidic conditions.

In certain embodiments where the selectivity-determining moiety is selected such that the bond is cleaved under basic conditions, the selectivity-determining moiety is an aminoalkylcarbonyloxyalkyl moiety. In certain embodiments, the selectivity-determining moiety has a structure

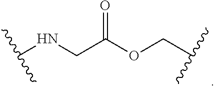

In certain embodiments where the selectivity-determining moiety is selected such that the bond is cleaved enzymatically, it may be selected such that a particular enzyme or class of enzymes cleaves the bond. In certain preferred such embodiments, the selectivity-determining moiety may be selected such that the bond is cleaved by a cathepsin, preferably cathepsin B.

In certain embodiments the selectivity-determining moiety comprises a peptide, preferably a dipeptide, tripeptide, or tetrapeptide. In certain such embodiments, the peptide is a dipeptide is selected from KF and FK, In certain embodiments, the peptide is a tripeptide is selected from GFA, GLA, AVA, GVA, GIA, GVL, GVF, and AVF. In certain embodiments, the peptide is a tetrapeptide selected from GFYA and GFLG, preferably GFLG.

In certain such embodiments, a peptide, such as GFLG, is selected such that the bond between the selectivity-determining moiety and the self-cyclizing moiety is cleaved by a cathepsin, preferably cathepsin B.

In certain embodiments, the selectivity-determining moiety is represented by

Formula A:

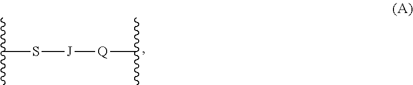

wherein

S a sulfur atom that is part of a disulfide bond;

J is optionally substituted hydrocarbyl; and

Q is O or NR¹³, wherein R¹³ is hydrogen or alkyl.

In certain embodiments, J may be polyethylene glycol, polyethylene, polyester, alkenyl, or alkyl. In certain embodiments, J may represent a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl. In certain embodiments, J may be substituted or unsubstituted lower alkylene, such as ethylene. For example, the selectivity-determining moiety may be

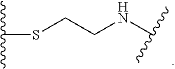

In certain embodiments, the selectivity-determining moiety is represented by Formula B:

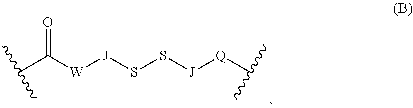

wherein

W is either a direct bond or selected from lower alkyl, NR¹⁴, S, O;

S is sulfur;

J, independently and for each occurrence, is hydrocarbyl or polyethylene glycol;

Q is O or NR¹³, wherein R¹³ is hydrogen or alkyl; and

R¹⁴ is selected from hydrogen and alkyl.

In certain such embodiments, J may be substituted or unsubstituted lower alkyl, such as methylene. In certain such embodiments, J may be an aryl ring. In certain embodiments, the aryl ring is a benzo ring. In certain embodiments W and S are in a 1,2-relationship on the aryl ring. In certain embodiments, the aryl ring may be optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, the aryl ring is optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{X}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, J, independently and for each occurrence, is polyethylene glycol, polyethylene, polyester, alkenyl, or alkyl.

In certain embodiments, independently and for each occurrence, the linker comprises a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl.

In certain embodiments, J, independently and for each occurrence, is substituted or unsubstituted lower alkylene. In certain embodiments, J, independently and for each occurrence, is substituted or unsubstituted ethylene.

In certain embodiments, the selectivity-determining moiety is selected from

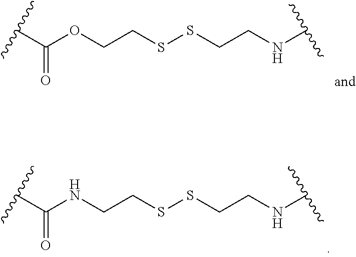

The selectivity-determining moiety may include groups with bonds that are cleavable under certain conditions, such as disulfide groups. In certain embodiments, the selectivity-determining moiety comprises a disulfide-containing moiety, for example, comprising aryl and/or alkyl group(s) bonded to a disulfide group. In certain embodiments, the selectivity-determining moiety has a structure

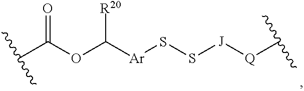

wherein

Ar is a substituted or unsubstituted benzo ring;

J is optionally substituted hydrocarbyl; and

Q is O or NR¹³,

wherein R¹³ is hydrogen or alkyl.

In certain embodiments, Ar is unsubstituted. In certain embodiments, Ar is a 1,2-benzo ring. For example, suitable moieties within Formula B include

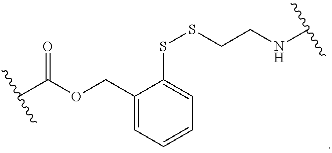

In certain embodiments, the self-cyclizing moiety is selected such that upon cleavage of the bond between the selectivity-determining moiety and the self-cyclizing moiety, cyclization occurs thereby releasing the therapeutic agent. Such a cleavage-cyclization-release cascade may occur sequentially in discrete steps or substantially simultaneously. Thus, in certain embodiments, there may be a temporal and/or spatial difference between the cleavage and the self-cyclization. The rate of the self-cyclization cascade may depend on pH, e.g., a basic pH may increase the rate of self-cyclization after cleavage. Self-cyclization may have a half-life after introduction in vivo of 24 hours, 18 hours, 14 hours, 10 hours, 6 hours, 3 hours, 2 hours, 1 hour, 30 minutes, 10 minutes, 5 minutes, or 1 minute.

In certain such embodiments, the self-cyclizing moiety may be selected such that, upon cyclization, a five- or six-membered ring is formed, preferably a five-membered ring. In certain such embodiments, the five- or six-membered ring comprises at least one heteroatom selected from oxygen, nitrogen, or sulfur, preferably at least two, wherein the heteroatoms may be the same or different. In certain such embodiments, the heterocyclic ring contains at least one nitrogen, preferably two. In certain such embodiments, the self-cyclizing moiety cyclizes to form an imidazolidone.

In certain embodiments, the self-cyclizing moiety has a structure

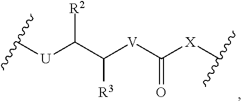

wherein

U is selected from NR¹ and S;

X is selected from O, NR^{S}, and S, preferably O or S;

V is selected from O, S and NR⁴, preferably O or NR⁴;

R² and R³ are independently selected from hydrogen, alkyl, and alkoxy; or R² and

R³ together with the carbon atoms to which they are attached form a ring; and

R¹, R⁴, and R⁵ are independently selected from hydrogen and alkyl.

In certain embodiments, U is NR¹ and/or V is NR⁴, and R¹ and R⁴ are independently selected from methyl, ethyl, propyl, and isopropyl. In certain embodiments, both R¹ and R⁴ are methyl. On certain embodiments, both R² and R³ are hydrogen. In certain embodiments R² and R³ are independently alkyl, preferably lower alkyl. In certain embodiments, R² and R³ together are —(CH₂)ₙ— wherein n is 3 or 4, thereby forming a cyclopentyl or cyclohexyl ring. In certain embodiments, the nature of R² and R³ may affect the rate of cyclization of the self-cyclizing moiety. In certain such embodiments, it would be expected that the rate of cyclization would be greater when R² and R³ together with the carbon atoms to which they are attached form a ring than the rate when R² and R³ are independently selected from hydrogen, alkyl, and alkoxy. In certain embodiments, U is bonded to the self-cyclizing moiety.

In certain embodiments, the self-cyclizing moiety is selected from

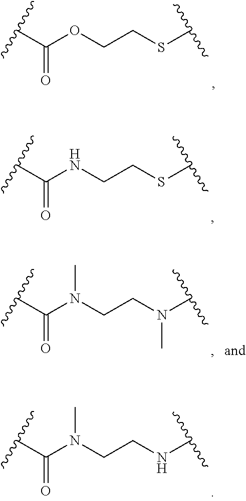

In certain embodiments, the selectivity-determining moiety may connect to the self-cyclizing moiety through carbonyl-heteroatom bonds, e.g., amide, carbamate, carbonate, ester, thioester, and urea bonds.

In certain embodiments, a proteasome inhibitor (such as a boronic acid containing proteasome inhibitor) is covalently attached to a polymer through a tether,

wherein the tether comprises a selectivity-determining moiety and a self-cyclizing moiety which are covalently attached to one another. In certain embodiments, the self-cyclizing moiety is selected such that after cleavage of the bond between the selectivity-determining moiety and the self-cyclizing moiety, cyclization of the self-cyclizing moiety occurs, thereby releasing the therapeutic agent. As an illustration, ABC may be a selectivity-determining moiety, and DEFGH maybe be a self-cyclizing moiety, and ABC may be selected such that enzyme Y cleaves between C and D. Once cleavage of the bond between C and D progresses to a certain point, D will cyclize onto H, thereby releasing therapeutic agent X, or a prodrug thereof.

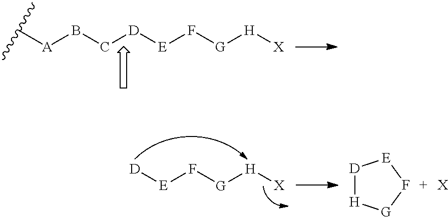

In certain embodiments, X may further comprise additional intervening components, including, but not limited to another self-cyclizing moiety or a leaving group linker, such as CO₂ or methoxymethyl, that spontaneously dissociates from the remainder of the molecule after cleavage occurs.

In some embodiments, a linker may be and/or comprise an alkylene chain, a polyethylene glycol (PEG) chain, polysuccinic anhydride, poly-L-glutamic acid, poly(ethyleneimine), an oligosaccharide, an amino acid (e.g., glycine or cysteine), an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, the linker group(s) of the present invention represent a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, the linker group represents a derivatized or non-derivatized amino acid (e.g., glycine or cysteine). In certain embodiments, linker groups with one or more terminal carboxyl groups may be conjugated to the polymer. In certain embodiments, one or more of these terminal carboxyl groups may be capped by covalently attaching them to a therapeutic agent, a targeting moiety, or a cyclodextrin moiety via an (thio)ester or amide bond. In still other embodiments, linker groups with one or more terminal hydroxyl, thiol, or amino groups may be incorporated into the polymer. In preferred embodiments, one or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. In certain embodiments, these (thio)ester, amide, (thio)carbonate or (thio)carbamates bonds may be biohydrolyzable, i.e., capable of being hydrolyzed under biological conditions.

In certain embodiments, a linker group represents a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, a linker group, e.g., between a proteasome inhibitor (such as a boronic acid containing proteasome inhibitor) and the CDP, comprises a self-cyclizing moiety. In certain embodiments, a linker group, e.g., between a proteasome inhibitor and the CDP, comprises a selectivity-determining moiety.

In certain embodiments as disclosed herein, a linker group, e.g., between a proteasome inhibitor (such as a boronic acid containing proteasome inhibitor) and the CDP, comprises a self-cyclizing moiety and a selectivity-determining moiety.

In certain embodiments as disclosed herein, the proteasome inhibitor (such as a boronic acid containing proteasome inhibitor) or targeting ligand is covalently bonded to the linker group via a biohydrolyzable bond (e.g., an ester, amide, carbonate, carbamate, or a phosphate).

In certain embodiments as disclosed herein, the CDP comprises cyclodextrin moieties that alternate with linker moieties in the polymer chain.

In certain embodiments, the linker moieties are attached to proteasome inhibitors (such as a boronic acid containing proteasome inhibitors) or prodrugs thereof that are cleaved under biological conditions.

In certain embodiments, at least one linker that connects the proteasome inhibitor (such as a boronic acid containing proteasome inhibitor) or prodrug thereof to the polymer comprises a group represented by the formula

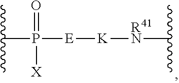

wherein

P is phosphorus;

O is oxygen;

E represents oxygen or NR⁴⁰;

K represents hydrocarbyl;

X is selected from OR⁴² or NR⁴³R⁴⁴; and

R⁴⁰, R⁴¹, R⁴², R⁴³, and R⁴⁴ independently represent hydrogen or optionally substituted alkyl.

In certain embodiments, E is NR⁴⁰ and R⁴⁰ is hydrogen.

In certain embodiments, K is lower alkylene (e.g., ethylene).

In certain embodiments, at least one linker comprises a group selected from

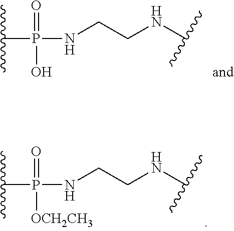

In certain embodiments, X is OR⁴².

In certain embodiments, the linker group comprises an amino acid or peptide, or derivative thereof (e.g., a glycine or cysteine).

In certain embodiments as disclosed herein, the linker is connected to the proteasome inhibitor (such as a boronic acid containing proteasome inhibitor) through a hydroxyl group. In certain embodiments as disclosed herein, the linker is connected to the proteasome inhibitor (such as a boronic acid containing proteasome inhibitor) through an amino group.

In certain embodiments, the linker group that connects to the proteasome inhibitor (such as a boronic acid containing proteasome inhibitor) may comprise a self-cyclizing moiety, or a selectivity-determining moiety, or both. In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, any of the linker groups may comprise a self-cyclizing moiety or a selectivity-determining moiety, or both. In certain embodiments, the selectivity-determining moiety may be bonded to the self-cyclizing moiety between the self-cyclizing moiety and the polymer.

In certain embodiments, any of the linker groups may independently be or include an alkyl chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkyl chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, any of the linker groups may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR¹, O or S), —OC(O)—, —C(═O)O—, —NR¹—, —NR¹CO—, —C(O)NR¹—, —S(O)ₙ— (wherein n is 0, 1, or 2), —OC(O)—NR¹—, —NR¹—C(O)—NR¹—, NR¹—C(NR¹)—NR¹—, and —B(OR¹)—; and R¹, independently for each occurrence, is H or lower alkyl.

In certain embodiments, the present invention contemplates a CDP, wherein a plurality of proteasome inhibitors (such as a boronic acid containing proteasome inhibitor, e.g., bortezomib) are covalently attached to the polymer through attachments that are cleaved under biological conditions to release the therapeutic agents as discussed above, wherein administration of the polymer to a subject results in release of the therapeutic agent over a period of at least 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month.

In some embodiments, the conjugation of the proteasome inhibitor (such as a boronic acid containing proteasome inhibitor, e.g., bortezomib) to the CDP improves the aqueous solubility of the proteasome inhibitor and hence the bioavailability. Accordingly, in one embodiment of the invention, the proteasome inhibitor (such as a boronic acid containing proteasome inhibitor, e.g., bortezomib) has a log P>0.4, >0.6, >0.8, >1, >2, >3, >4, or even >5.

The CDP-proteasome inhibitor (such as a boronic acid containing proteasome inhibitor, e.g., bortezomib) of the present invention preferably has a molecular weight in the range of 10,000 to 500,000; 30,000 to 200,000; or even 70,000 to 150,000 amu.

In certain embodiments, the present invention contemplates attenuating the rate of release of the proteasome inhibitor (such as a boronic acid containing proteasome inhibitor, e.g., bortezomib) by introducing various tether and/or linking groups between the therapeutic agent and the polymer. Thus, in certain embodiments, the CDP-proteasome inhibitor (such as a boronic acid containing proteasome inhibitor) conjugates, e.g., a CDP-bortezomib conjugate, of the present invention are compositions for controlled delivery of the proteasome inhibitor, such as a boronic acid containing proteasome inhibitor, e.g., bortezomib.

### Proteasome Inhibitor

The proteasome inhibitors in the CDP-proteasome inhibitor conjugate described herein, e.g., CDP-proteasome inhibitor conjugate represented by formulas (I)-(VIII), include pharmaceutically active agents, preferably a proteasome inhibitor comprising a boronic acid moiety or a derivative thereof described herein, e.g., RB(OH)₂ or its boronic acid derivative described herein. Additionally, proteasome inhibitors include peptide aldehyde proteasome inhibitors such as those disclosed in Stein et al. U.S. Pat. No. 5,693,617 (1997), International patent publications WO 95/24914, published Sep. 21, 1995 and Siman et al. WO 91/13904 published Sep. 19, 1991; Iqbal et al. J. Med. Chem. 38:2276-2277 (1995), as well as Bouget et al. Bioorg. Med. Chem. 17:4881-4889 (2003), each of which is hereby incorporated by reference in its entirety, including all compounds and formulae disclosed therein.

Further, proteasome inhibitors include lactacystin and lactacycstin analogs which have been disclosed in Fentany et al., U.S. Pat. No. 5,756,764 (1998), and U.S. Pat. No. 6,147,223 (2000), Schreiber et al U.S. Pat. No. 6,645,999 (2003), and Fenteany et al. Proc. Natl. Acad. Sci. USA (1994) 91:3358, each of which is hereby incorporated by reference in its entirety, including all compounds and formulae disclosed therein. Additionally, synthetic peptide vinyl sulfone proteasome inhibitors and epoxyketone proteasome inhibitors are also included in the present invention. See, e.g., Bogyo et al., Proc. Natl. Acad. Sci. 94:6629 (1997); Spaltenstein et al. Tetrahedron Lett. 37:1343 (1996); Meng L; Proc. Natl. Acad Sci 96: 10403 (1999); and Meng L H, Cancer Res 59: 2798 (1999), each of which is hereby incorporated by reference in its entirety.

Still further, natural compounds have been recently shown to have proteasome inhibition activity are included in the present invention. For example, TMC-95A, a cyclic peptide, or Gliotoxin, both fungal metabolites or polyphenols compounds found in green tea have been identified as proteasome inhibitors. See, e.g., Koguchi Y, Antibiot (Tokyo) 53:105. (2000); Kroll M, Chem Biol 6:689 (1999); and Nam S, J. Biol Chem 276: 13322 (2001), each of which is hereby incorporated by reference in its entirety.

### Exemplary CDP-Proteasome Inhibitor Conjugates

The CDP-proteasome inhibitor conjugate (such as a boronic acid containing proteasome inhibitor) of the invention comprises a proteasome inhibitor (such as a boronic acid containing proteasome inhibitor, e.g., bortezomib) covalently linked to a CDP described herein. In one embodiment, the proteasome inhibitor is a pharmaceutically active agent, preferably comprises a boronic acid moiety or a boronic acid derivative described herein.

As used herein, a boronic acid derivative is represented by R—B(Y)₂, wherein each Y is a group that is readily displaced by an amine or alcohol group on the liker L to form a covalent bond. Examples of boronic acid derivatives include boronic ester (e.g., RB(O-alkyl)₂), boronic amides (e.g., RB(N(alkyl)₂)₂), alkoxyboranamine (e.g., RB(O-alkyl)(N(alkyl)₂); and boronic acid anhydride. Mixed boronic acid derivatives are also included, such as RB(O-alkyl)(N(alkyl)₂).

In another embodiment, for the CDP-proteasome inhibitor conjugate represented by formulas (I)-(VIII), D is —B—R, wherein R is as described in RB(OH)₂ or RB(Y)₂ described herein; RB(OH)₂ is a pharmaceutically active agent, preferably a proteasome inhibitor comprising a boronic acid moiety (e.g., bortezomib); and L is a linker comprising two functional groups that bind to the boron atom and upon binding to RB(OH)₂ or RB(Y)₂, the two functional groups displace the two —OH groups in RB(OH)₂ or the two —Y groups in RB(Y)₂ that are attached to the boron atom to form a moiety represented by the following formula:

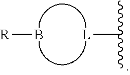

In a first embodiment, the CDP-proteasome conjugate is represented by a formula selected from:

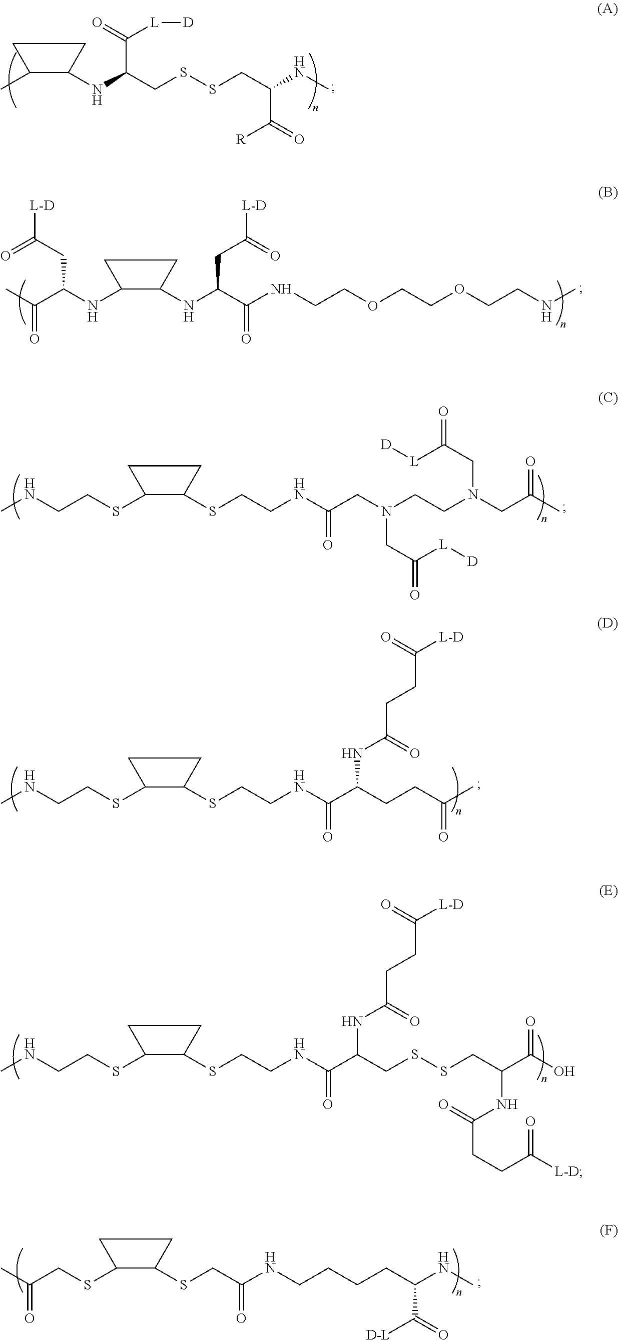

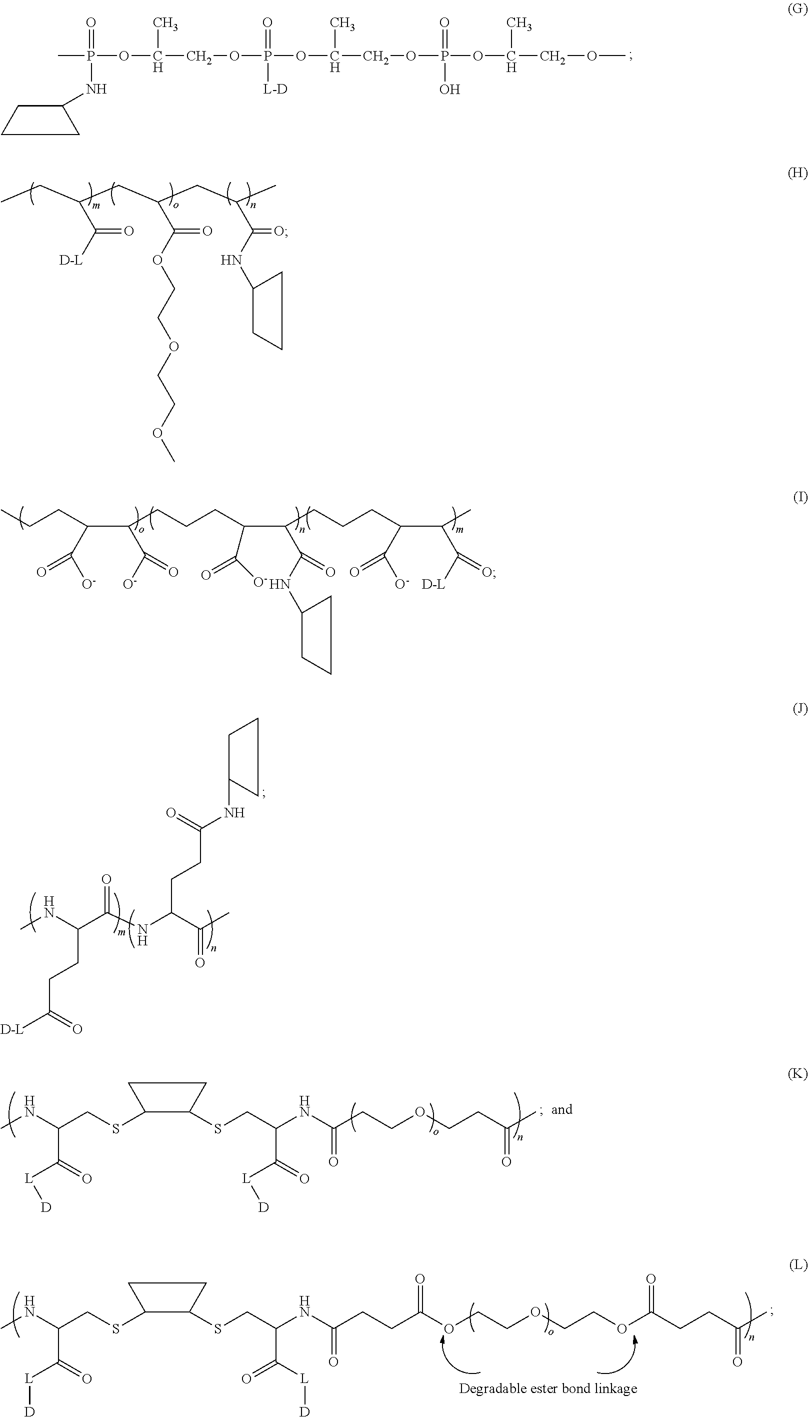

wherein:

each m and n is independent an integer from 1 to 100;

o is an integer from 1 to 1000;

L is a linker described in Formulas (I)-(VIII); and

D is —B—R, wherein R is as described in RB(OH)₂ or RB(Y)₂ described above.

In another embodiment, the L-D moiety in formulas (A)-(L) is represented by the following formula:

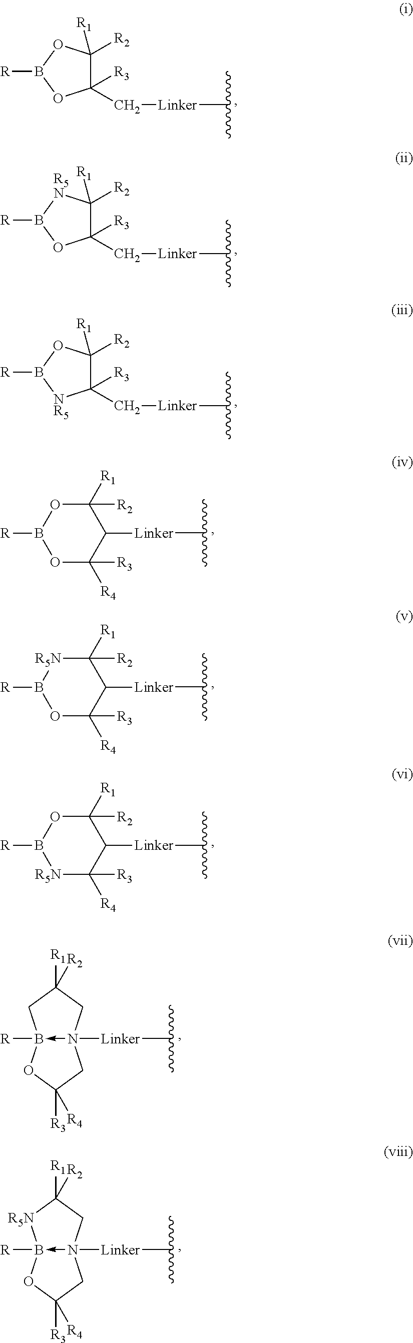

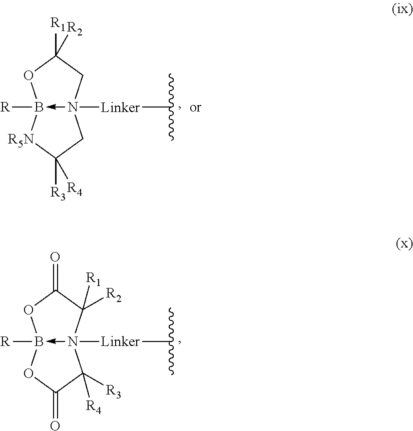

wherein:

R is the non-boronic acid moiety in R—B(OH)₂ or R is as described in a boronic acid derivative RB(Y)₂ described herein;

RB(OH)₂ is a pharmaceutically active agent, preferably a proteasome inhibitor comprising a boronic acid moiety, such as bortezomib;

RB(Y)₂ is a pharmaceutically active agent, preferably a proteasome inhibitor such as a proteosome inhibitor comprising a boronic acid derivative;

R₁, R₂, R₃, R₄ and R₅ are each independently —H or a (C₁-C₅)alkyl; and

linker is a linker group comprising an amino terminal group.

In a second embodiment, for CDP-proteasome inhibitor conjugate represented by formulas (A)-(L), the L-D moiety is represented by a formula selected from:

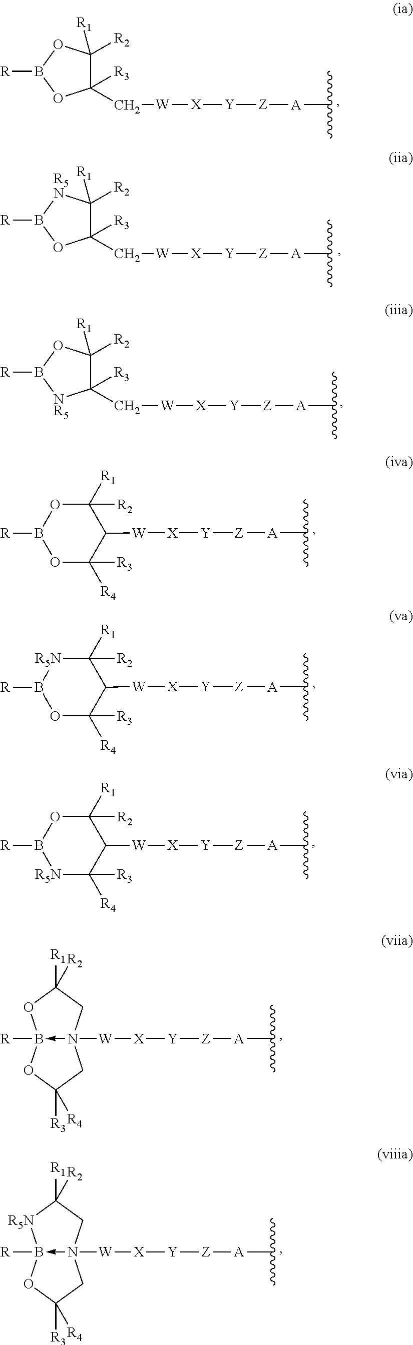

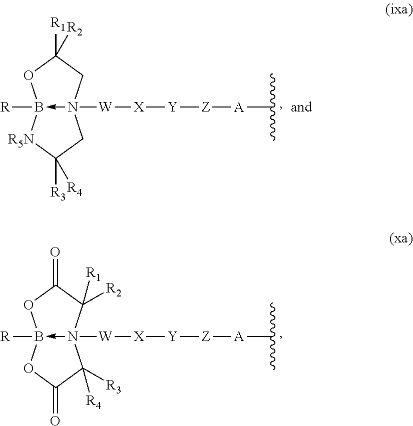

wherein:

R₁, R₂, R₃, R₄ and R₅ are each independently —H or a (C₁-C₅)alkyl;

R is as described in RB(OH)₂ or RB(Y)₂ described above;

W is —(CH₂)ₘ—, —O— or —N(R₅′)—, when the polymer-agent conjugate is represented by structural formulas (ia)-(via); or

W is —(CH₂)ₘ—, when the polymer-agent conjugate is represented by structural formulas (viia)-(xa);

X is a bond when W is —(CH₂)ₘ— and X is —C(═O)— when W is —O—, or —N(R₅′);

Y is a bond, —O—, or —N(R₅′)—;

Z is represented by the following structural formula:

—(CH₂)ₚ-Q—(CH₂)_{q}-E-;

E is a bond, aryl (e.g., phenyl) or heteroaryl (e.g., pyridyl, furyl or furanyl, imidazolyl, benzimidazolyl, pyrimidinyl, thiophenyl or thienyl, quinolinyl, indolyl and thiazolyl);

Q is a bond, —O—, —N(R₅′)—, —N(R₅′)—C(═O)—O—, —O—C(═O)—N(R₅′)—, —OC(═O)—, —C(═O)—O—, —S—S—, —(O—CH₂—CH₂)ₙ— or

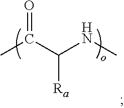

Rₐ is a side chain of a naturally occurring amino acid or an analog thereof;

A is —N(R₅′)—, or A is a bond when Q is

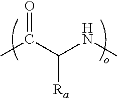

and q is 0;

R₅′ is —H or (C₁-C₆)alkyl;

m, p, q are each an integer from 0 to 10;

n is an integer from 1 to 10; and

o is an integer from 1 to 10, provided when Y is —O— or —N(R₅′)— and Q is —O—, —N(R₅′)—, —(O—CH₂—CH₂)ₙ—, —N(R₅′)—C(═O)—O—, —O—C(═O)—N(R₅′)—, —OC(═O)— or —S—S—, then p is an integer from 2 to 10; when Q is —O—, —N(R₅′)—, —N(R₅′)—C(═O)—O—, —O—C(═O)—N(R₅′)—, —OC(═O)—, —C(═O)—O—, or —S—S— and E is a bond, then q is an integer from 2 to 10; when Y is —O— or —N(R₅′)—, Q and E are both a bond, then p+q≧2; when W is —O— or —N(R₅′)—, Y, Q and E are all bond, then p+q≧1; and when W is —O— or —N(R₅′)—, Y is a bond, and Q is —N(R₅′)—C(═O)—O—, —O—C(═O)—N(R₅′)—, —OC(═O)—, —C(═O)—O—, —S—S— or —(O—CH₂—CH₂)ₙ—, then p is an integer from 2 to 10.

In one embodiment, Z is a bond or —(CH₂)ₙ—, wherein r is an integer from 1 to 10.

In a third embodiment, for CDP-proteasome inhibitor conjugate described in the second embodiment, the linker (i.e. —W—X—Y—Z-A) is represented by any one of the following formula:

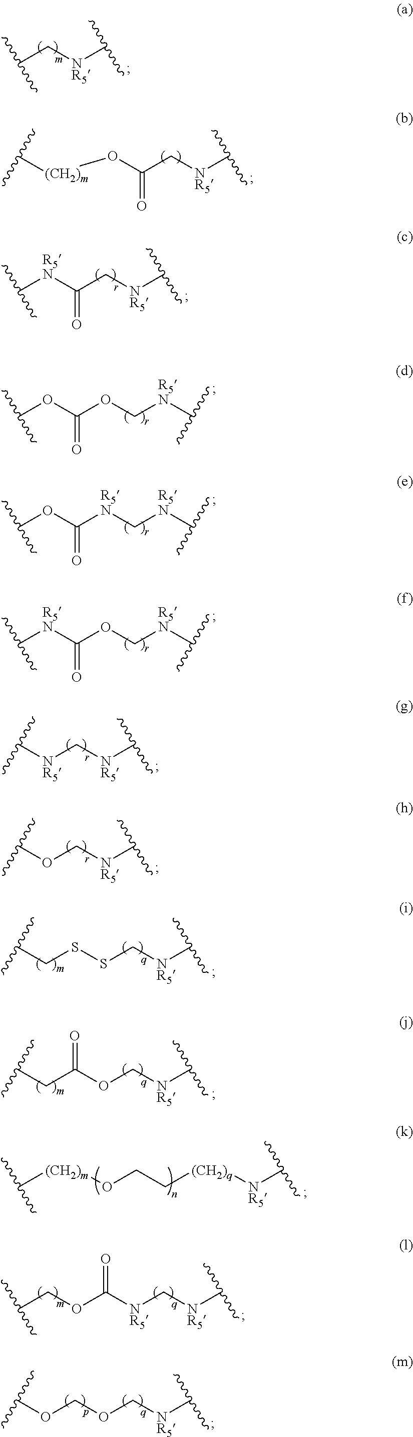

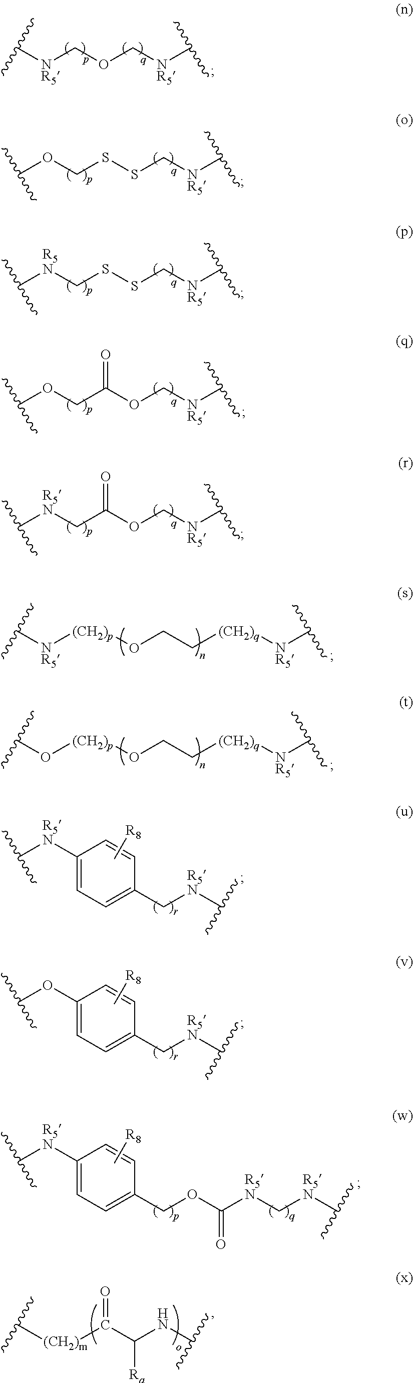

wherein R₅′ is —H or (C₁-C₆)alkyl; Rₐ is a side chain of a naturally occurring amino acid or an analog thereof; R₈ is a substituent; n is an integer from 1 to 10; r is an integer from 1 to 10; m, p and q are each an integer from 0 to 10; and o is an integer from 1 to 10. For formulas (d)-(h), r is an integer from 2 to 10. For formulas (i), (j) and (1), q is an integer from 2 to 10. For formulas (m)-(p), p and q are each an integer from 2 to 10. For formulas (q) and (r), p is an integer from 1 to 10 and q is an integer from 2 to 10. For formulas (s) and (t), p is an integer from 2 to 10. For formula (w), q is an integer from 2 to 10. More specifically, R₈ is selected from H, halo, —CN, —NO₂, —OH, (C₁-C₆)alkyl, halo(C₁-C₆)alkyl, hydroxy(C₁-C₆)alkyl, (C₁-C₆)alkoxy, halo(C₁-C₆)alkoxy, (C₁-C₃)alkoxy(C₁-C₃)alkyl and —NR₉R₁₀; wherein R₉ and R₁₀ are each independently H, (C₁-C₆)alkyl, halo(C₁-C₆)alkyl, (C₁-C₆)alkoxy, halo(C₁-C₆)alkoxy, (C₁-C₃)alkoxy(C₁-C₃)alkyl.

In a fourth embodiment, for CDP-proteasome inhibitor conjugate described in the third embodiment, the linker (i.e., —W—X—Y—Z-A) is represented by any one of the following formulas:

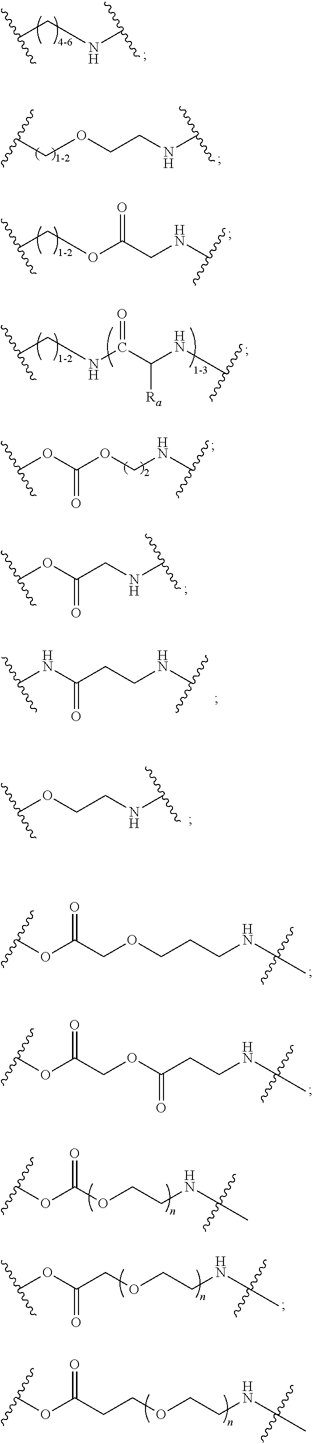

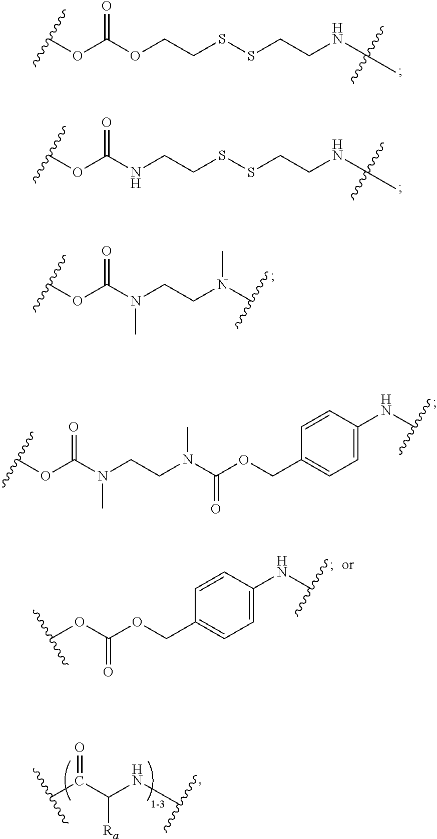

wherein n is an integer from 2 to 5; and Rₐ is a side chain of a naturally occurring amino acid or an analog thereof.

In a fifth embodiment, for the CDP-proteasome inhibitor conjugate described in the first embodiment, the linker is represented by formulas (AA1), (BB1) or (CC1):

—(CH₂)ₘ—O—CH₂—O—(CH₂)_{q}—N(R₅)— (AA1),

—(CH₂)ₘ—O—(CH₂)ₚ—O—CH₂—N(R₅)— (BB1)

—(CH₂)ₘ—(CH₂)ₚ—O—CH₂—N(R₅)— (CC1)

wherein m is an integer from 0 to 10; q is an integer from 2 to 10; p is an integer from 0 to 10 for structural formula (CC1) and p is an integer from 2 to 10 for structural formula (BB1).

In a sixth embodiment, for CDP-proteasome inhibitor conjugate of formulas (A)-(L) described in the first embodiment, the L-D moiety is as described in Table 2.

In a seventh embodiment, the CDP-proteasome inhibitor conjugate is represented by the following formula:

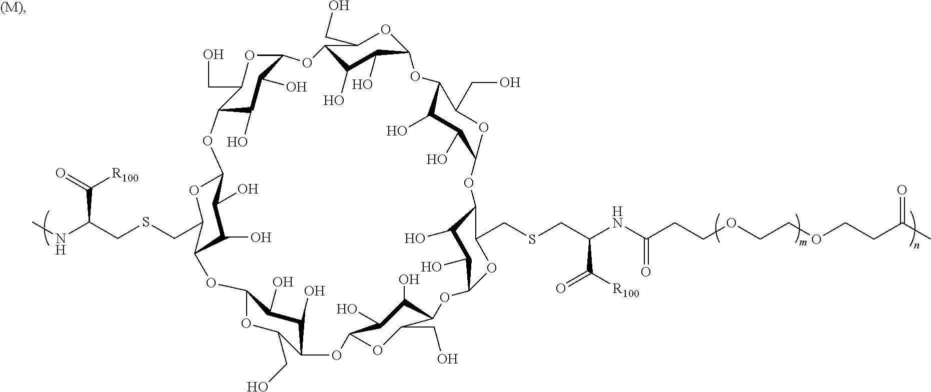

wherein n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); and R₁₀₀ is —OH or a group comprising a —B—R moiety, wherein R is as described in RB(OH)₂ or RB(Y)₂ described above. At least one R₁₀₀ in the conjugate is a group comprising a —B—R moiety. Alternatively, the conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a group comprising a —B—R moiety per repeat unit. In one embodiment, at least one R₁₀₀ in the conjugate is a group comprising a —B—R moiety and R is represented by the following structural formula:

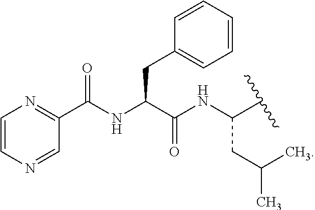

Alternatively, the conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a group comprising a —B—R moiety per repeat unit and R is represented by the following structural formula:

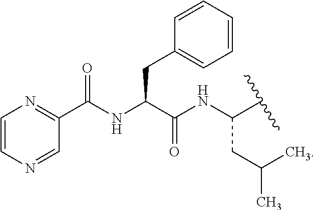

In an eighth embodiment, the CDP-proteasome inhibitor conjugate is represented by formula (M):

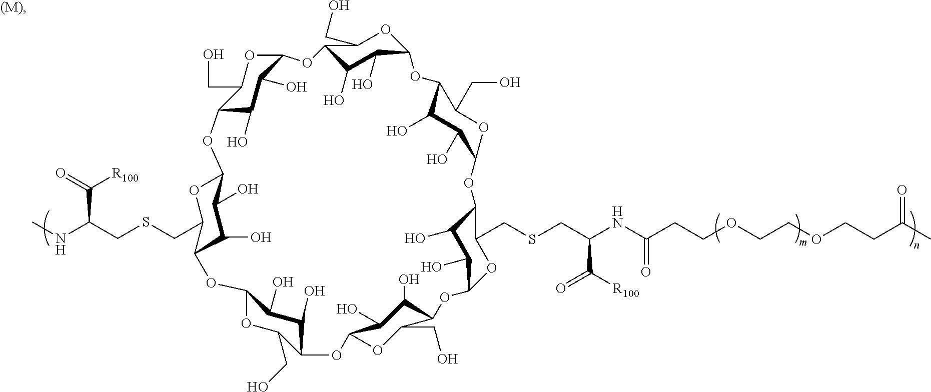

wherein n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by a formula selected from formulas (i)-(x). At least one R₁₀₀ group in the conjugate is a group represented by a formula selected from formulas (i)-(x). Alternatively, the conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a formula selected from formulas (i)-(x) per repeat unit.

In a ninth embodiment, for the CDP-proteasome inhibitor conjugate represented by formula (M), n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by a formula selected from formulas (i)-(x). At least one R₁₀₀ group in the conjugate is a group represented by a formula selected from formulas (i)-(x); and R in formulas (i)-(x) is as described in RB(OH)₂ or RB(Y)₂ described above. More specifically, at least one R₁₀₀ group in the conjugate is a group represented by a formula selected from formulas (i)-(x); and R in formulas (i)-(x) is represented by the following structural formula:

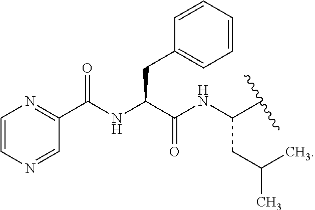

Alternatively, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a formula selected from formulas (i)-(x) per repeat unit; and R in formulas (i)-(x) is as described in RB(OH)₂ or RB(Y)₂ described above. More specifically, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a formula selected from formulas (i)-(x) per repeat unit; and R in formulas (i)-(x) is represented by the following structural formula:

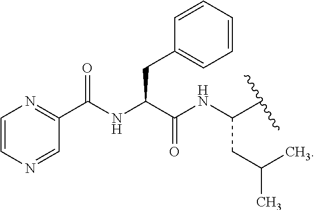

In a tenth embodiment, for the CDP-proteasome inhibitor conjugate represented by formula (M), n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by a formula selected from formulas (ia)-(xa). At least one R₁₀₀ group in the conjugate is a group represented by a formula selected from formulas (ia)-(xa). Alternatively, the conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a formula selected from formulas (ia)-(xa) per repeat unit.

In an eleventh embodiment, for the CDP-proteasome inhibitor conjugate represented by formula (M), n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by a formula selected from formulas (ia)-(xa). At least one R₁₀₀ group in the conjugate is a group represented by a formula selected from formulas (ia)-(xa); and R in formulas (ia)-(xa) is as described in RB(OH)₂ or RB(Y)₂ described above. More specifically, at least one R₁₀₀ group in the conjugate is a group represented by a formula selected from formulas (ia)-(xa); and R in formulas (i)-(x) is represented by the following structural formula:

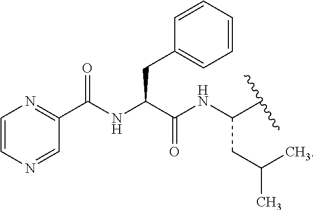

Alternatively, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a formula selected from formulas (ia)-(xa) per repeat unit; and R in formulas (ia)-(xa) is as described in RB(OH)₂ or RB(Y)₂ described above. More specifically, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by a formula selected from formulas (ia)-(xa) per repeat unit; and R in formulas (ia)-(xa) is represented by the following structural formula:

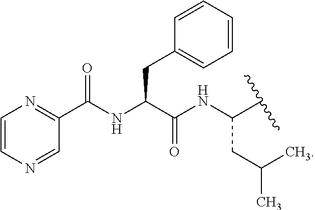

In a twelfth embodiment, for the CDP-proteasome inhibitor conjugate represented by formula (M), n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by formula (ia). At least one R₁₀₀ group in the conjugate is a group represented by formula (1a) and the group —W—X—Y—Z-A in R₁₀₀ represented by formula (ia) is represented by a formula selected from formulas (a)-(x) described in the 3^{rd} embodiment and formulas (AA1), (BB1) and (CC1) described in the 5^{th} embodiment. Alternatively, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by formula (ia) per repeat unit; and the group —W—X—Y—Z-A in R₁₀₀ represented by formula (ia) is represented by a formula selected from formulas (a)-(x) described in the third embodiment and formulas (AA1), (BB1) and (CC1) described in the fifth embodiment.

Alternatively, in the twelfth embodiment described above, R₁₀₀ is represented by formula (iia) instead of formula (ia). Alternatively, in the twelfth embodiment described above, R₁₀₀ is represented by formula (iiia) instead of formula (ia). Alternatively, in the twelfth embodiment above, R₁₀₀ is represented by formula (iva) instead of formula (ia). Alternatively, in the twelfth embodiment described above, R₁₀₀ is represented by formula (va) instead of formula (ia). Alternatively, in the 12^{th} embodiment described above, R₁₀₀ is represented by formula (via) instead of formula (ia). Alternatively, in the twelfth embodiment described above, R₁₀₀ is represented by formula (viia) instead of formula (ia). Alternatively, in the twelfth embodiment described above, R₁₀₀ is represented by formula (viiia) instead of formula (ia). Alternatively, in the 12^{th embodiment described above, R}₁₀₀ is represented by formula (ixa) instead of formula (ia). Alternatively, in the twelfth embodiment described above, R₁₀₀ is represented by formula (xa) instead of formula (ia).

In a thirteenth embodiment, for the CDP-proteasome inhibitor conjugate represented by formula (M), n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by (ia). At least one R₁₀₀ group in the conjugate is a group represented by (ia); the group —W—X—Y—Z-A in formula (ia) is represented by a formula selected from formulas (a)-(x) described in the 3^{rd} embodiment and formulas (AA1), (BB1) and (CC1) described in the 5^{th} embodiment; and R in R₁₀₀ represented by formula (ia) is as describe in RB(OH)₂ or RB(Y)₂ described above. More specifically, at least one R₁₀₀ group in the conjugate is a group represented by formula (ia); the group —W—X—Y—Z-A in R₁₀₀ represented by formula (ia) is represented by a formula selected from formulas (a)-(x) described in the third embodiment and formulas (AA1), (BB1) and (CC1) described in the fifth embodiment; and R in R₁₀₀ represented by formula (ia) is represented by the following structural formula:

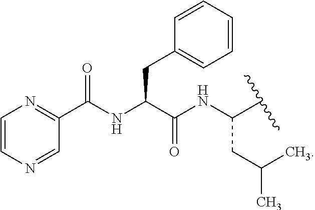

Alternatively, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by formula (ia) per repeat unit; the group —W—X—Y—Z-A in R₁₀₀ represented by formula (ia) is represented by a formula selected from formulas (a)-(x) described in the third embodiment and formulas (AA1), (BB1) and (CC1) described in the fifth embodiment; and R in R₁₀₀ represented by formula (ia) is as described in RB(OH)₂ or RB(Y)₂ described above. More specifically, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by formula (ia) per repeat unit; the group —W—X—Y—Z-A in R₁₀₀ represented by formula (ia) is represented by a formula selected from formulas (a)-(x) described in the third embodiment and formulas (AA1), (BB1) and (CC1) described in the fifth embodiment; and R in R₁₀₀ represented by formula (ia) is represented by the following structural formula:

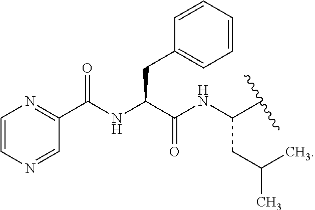

Alternatively, in the thirteenth embodiment described above, R₁₀₀ is represented by formula (iia) instead of formula (ia). Alternatively, in the thirteenth embodiment described above, R₁₀₀ is represented by formula (iiia) instead of formula (ia). Alternatively, in the thirteenth embodiment above, R₁₀₀ is represented by formula (iva) instead of formula (ia). Alternatively, in the thirteenth embodiment described above, R₁₀₀ is represented by formula (va) instead of formula (ia). Alternatively, in the thirteenth embodiment described above, R₁₀₀ is represented by formula (via) instead of formula (ia). Alternatively, in the thirteenth embodiment described above, R₁₀₀ is represented by formula (viia) instead of formula (ia). Alternatively, in the thirteenth embodiment described above, R₁₀₀ is represented by formula (viiia) instead of formula (ia). Alternatively, in the thirteenth embodiment described above, R₁₀₀ is represented by formula (ixa) instead of formula (ia). Alternatively, in the thirteenth embodiment described above, R₁₀₀ is represented by formula (xa) instead of formula (ia).

In a fourteenth embodiment, for the CDP-proteasome inhibitor conjugate represented by formula (M), n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by formula (ia). At least one R₁₀₀ group in the conjugate is a group represented by formula (ia) and the group —W—X—Y—Z-A in R₁₀₀ represented by formula (ia) is represented by a formula selected from the formulas described in the fourth embodiment. Alternatively, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by formula (ia) per repeat unit; and the group —W—X—Y—Z-A in R₁₀₀ represented by formula (ia) is represented by a formula selected from the formulas described in the 4^{th} embodiment.

Alternatively, in the fourteenth embodiment described above, R₁₀₀ is represented by formula (iia) instead of formula (ia). Alternatively, in the fourteenth embodiment described above, R₁₀₀ is represented by formula (iiia) instead of formula (ia). Alternatively, in the fourteenth embodiment above, R₁₀₀ is represented by formula (iva) instead of formula (ia). Alternatively, in the fourteenth embodiment described above, R₁₀₀ is represented by formula (va) instead of formula (ia). Alternatively, in the fourteenth embodiment described above, R₁₀₀ is represented by formula (via) instead of formula (ia). Alternatively, in the fourteenth embodiment described above, R₁₀₀ is represented by formula (viia) instead of formula (ia). Alternatively, in the fourteenth embodiment described above, R₁₀₀ is represented by formula (viiia) instead of formula (ia). Alternatively, in the fourteenth embodiment described above, R₁₀₀ is represented by formula (ixa) instead of formula (ia). Alternatively, in the fourteenth embodiment described above, R₁₀₀ is represented by formula (xa) instead of formula (ia).

In a fifteenth embodiment, for the CDP-proteasome inhibitor conjugate represented by formula (M), n is an integer from 1 to 100 (e.g., n is an integer from 4 to 80, from 4 to 50, from 4 to 30 or from 4 to 20, or n is 4, 5, 6, 7, 8, 9, 10, 11, 12, 13, 14, 15, 16, 17, 18, 19 or 20); m is an integer from 1 to 1000 (e.g., m is an integer from 1 to 200, from 1 to 100, from 1 to 80, from 2 to 80, from 5 to 70, from 10 to 50, or from 20 to 40); R₁₀₀ is —OH or a group represented by formula (ia). At least one R₁₀₀ group in the conjugate is a group represented by formula (ia); the group —W—X—Y—Z-A in R₁₀₀ represented by formula (ia) is represented by a formula selected from the formulas described in the fourth embodiment; and R in R₁₀₀ represented by formula (ia) is as described in RB(OH)₂ or RB(Y)₂ described above. More specifically, at least one R₁₀₀ group in the conjugate is a group represented by formula (ia); the group —W—X—Y—Z-A in R₁₀₀ represented by formula (ia) is represented by a formula selected from the formulas described in the fourth embodiment; and R in R₁₀₀ represented by formulas (ia) is represented by the following structural formula:

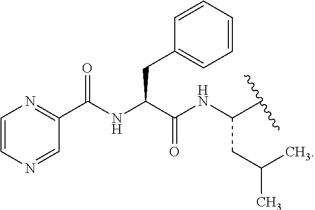

Alternatively, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by formula (ia) per repeat unit; the group —W—X—Y—Z-A in R₁₀₀ represented by formula (ia) is represented by a formula selected from the formulas described in the fourth embodiment; and R in R₁₀₀ represented by formula (ia) is as described in RB(OH)₂ or RB(Y)₂ described above. More specifically, the CDP-proteasome inhibitor conjugate represented by formula (M) comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 R₁₀₀ groups represented by formula (ia) per repeat unit; the group —W—X—Y—Z-A in R₁₀₀ represented by formula (ia) is represented by a formula selected from the formulas described in the fourth embodiment; and R in R₁₀₀ represented by formula (ia) is represented by the following structural formula:

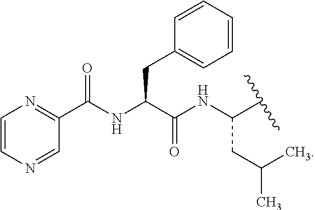

Alternatively, in the fifteenth embodiment described above, R₁₀₀ is represented by formula (iia) instead of formula (ia). Alternatively, in the fifteenth embodiment described above, R₁₀₀ is represented by formula (iiia) instead of formula (ia). Alternatively, in the fifteenth embodiment above, R₁₀₀ is represented by formula (iva) instead of formula (ia). Alternatively, in the fifteenth embodiment described above, R₁₀₀ is represented by formula (va) instead of formula (ia). Alternatively, in the fifteenth embodiment described above, R₁₀₀ is represented by formula (via) instead of formula (ia). Alternatively, in the fifteenth embodiment described above, R₁₀₀ is represented by formula (viia) instead of formula (ia). Alternatively, in the fifteenth embodiment described above, R₁₀₀ is represented by formula (viiia) instead of formula (ia). Alternatively, in the fifteenth embodiment described above, R₁₀₀ is represented by formula (ixa) instead of formula (ia). Alternatively, in the fifteenth embodiment described above, R₁₀₀ is represented by formula (xa) instead of formula (ia).

In the seventh through the fifteenth embodiments, n is preferably an integer from 4 to 20 and m is an integer from 1 to 1000; n is an integer from 4 to 80 and m is an integer from 1 to 200; n is an integer from 4 to 50 and m is an integer from 1 to 100; n is an integer from 4 to 30 and m is an integer from 1 to 80; n is an integer from 4 to 20 and m is an integer from 2 to 80; n is an integer from 4 to 20 and m is an integer from 5 to 70; n is an integer from 4 to 20 and m is an integer from 10 to 50; or n is an integer from 4 to 20 and m is an integer from 20-40.

In one embodiment, for the CDP-proteasome inhibitor conjugate described in any one of first to fifteenth embodiments, R in formulas (i)-(x) and (ia)-(xa) is represented by the following structural formula:

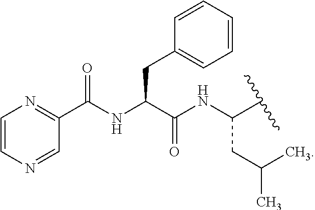

In one embodiment, for the CDP-proteasome inhibitor conjugate described in any one of first to fifteenth embodiments, RB(OH)₂ or RB(Y)₂ is as described in WO 91/13904, U.S. Pat. Nos. 5,780,454, 6,066,730, 6,083,903, 6,297,217, 6,465,433, 6,548,668, 6,617,317, 6,699,835, 6,713,446, 6,747,150, 6,958,319, 7,109,323, 7,119,080, 7,442,830, 7,531,526 and U.S. Published Applications 2009/0247731, 2009/099132, 2009/0042836, 2008/0132678, 2007/0282100, 2006/0122390, 2005/0282742, 2005/0240047, 2004/0167332, 2004/0138411, 2003/0199561, 2002/0188100 and 2002/0173488. Each of these patent documents is incorporated by reference in its entirety.

In one embodiment, for the CDP-proteasome inhibitor conjugates described in any one of first to fifteenth embodiments, RB(OH)₂ or RB(Y)₂ is represented by formula (1a):

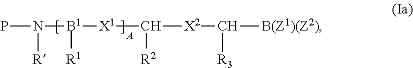

or a pharmaceutically acceptable salts thereof, wherein:

P is hydrogen or an amino-group-protecting moiety;

B¹, at each occurrence, is independently one of N or CH;
X¹, at each occurrence, is independently one of —C(O)—NH—, —CH₂—NH—, —CH(OH)—CH₂—, —CH(OH)—CH(OH)—, —CH(OH)—CH₂—NH—, —CH═CH—, —C(O)CH₂—, —SO₂—NH—, —SO₂—CH₂— or —CH(OH)—CH₂—C(O)—NH—, provided that when B¹ is N, then the X¹ attached to said B¹ is —C(O)—NH—;
X² is one of —C(O)—NH—, —CH(OH)—CH₂—, —CH(OH)—CH(OH)—, —C(O)—CH₂—, —SO₂—NH—, —SO₂—CH₂— or —CH(OH)—CH₂—C(O)—NH—;
R′ is hydrogen or alkyl, or R forms together with the adjacent R¹, or when A is zero, forms together with the adjacent R², a nitrogen-containing mono-, bi- or tri-cyclic, saturated or partially saturated ring system having 4-14 ring members, that can be optionally substituted by one or two of keto, hydroxy, alkyl, aryl, aralkyl, alkoxy or aryloxy;
R¹, at each occurrence, is independently one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle can be optionally substituted;
R² is one of hydrogen, alkyl, cycloalkyl, aryl, a 5-10 membered saturated, partially unsaturated or aromatic heterocycle or —CH—R⁵, where the ring portion of any of said aryl, aralkyl, alkaryl or heterocycle can be optionally substituted;
R³ is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle can be optionally substituted;
R⁵, in each instance, is one of aryl, aralkyl, alkaryl, cycloalkyl, a 5-10 membered saturated, partially unsaturated or aromatic heterocycle or —W—R⁶, where W is a chalcogen and R⁶ is alkyl, where the ring portion of any of said aryl, aralkyl, alkaryl or heterocycle can be optionally substituted;
Z¹ and Z² are independently one of alkyl, hydroxy, alkoxy, or aryloxy, or together Z¹ and Z² form a moiety derived from a dihydroxy compound having at least two hydroxy groups separated by at least two connecting atoms in a chain or ring, said chain or ring comprising carbon atoms, and optionally, a heteroatom or heteroatoms which can be N, S, or O; and A is 0, 1, or 2.

In one embodiment, for formula (1a):

P is R′ or R⁷—C(═O)— or R⁷—SO₂—, wherein R⁷ selected from the group consisting of

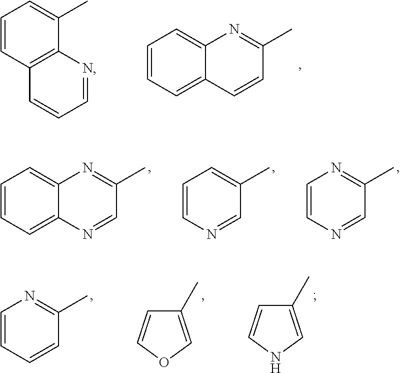

or P is

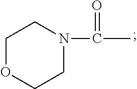

X₂ is selected from the group consisting of

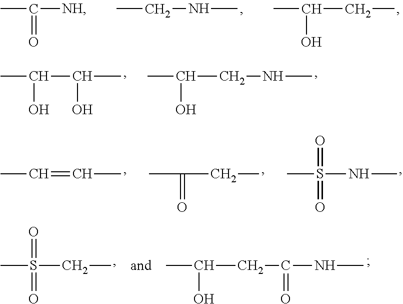

R′ is hydrogen or alkyl;

R₂ and R₃ are independently selected from the group consisting of hydrogen, alkyl, cycloalkyl, aryl, heterocycle and —CH₂—R₅, where R₅ is aryl, aralkyl, alkaryl, cycloalkyl, heterocycle or —Y—R₆,

where Y is a chalcogen, and R₆ is alkyl;
Z₁ and Z₂ are independently alkyl, hydroxy, alkoxy, aryloxy, or together form a dihydroxy compound having at least two hydroxy groups separated by at least two connecting atoms in a chain or ring, said chain or ring comprising carbon atoms, and optionally, a heteroatom or heteroatoms which can be N, S, or O; and A is 0.

In another embodiment, for structural formula (1a):

P is R₇—C(O)— or R₇—SO₂—, where R₇ is pyrazinyl;

X₂ is —C(O)—NH—;

R′ is hydrogen or alkyl;

R₂ and R₃ are independently hydrogen, alkyl, cycloalkyl, aryl, or —CH₂—R₅;

R₅ in each instance, is one of aryl, aralkyl, alkaryl, cycloalkyl, or —W—R₆, where W is a chalcogen and R₆ is alkyl;

where the ring portion of any of said aryl, aralkyl, or alkaryl in R₂, R₃ and R₅ can be optionally substituted by one or two substituents independently selected from the group consisting of C₁₋₆ alkyl, C₃₋₈ cycloalkyl, C₁₋₆ alkyl(C₃₋₈)cycloalkyl, C₂₋₈ alkenyl, C₂₋₈ alkynyl, cyano, amino, C₁₋₆ alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀ aryl(C₁₋₆)alkyl, C₆₋₁₀ aryl(C₁₋₆)alkoxy, hydroxy, C₁₋₆ alkylthio, C₁₋₆alkylsulfinyl, C₁₋₆ alkylsulfonyl, C₆₋₁₀ arylthio, C₆₋₁₀ arylsulfinyl, C₆₋₁₀ arylsulfonyl, C₆₋₁₀ aryl, C₁₋₆ alkyl(C₆₋₁₀) aryl, and halo(C₆₋₁₀)aryl;
Z₁ and Z₂ are independently one of hydroxy, alkoxy, or aryloxy, or together Z₁ and Z₂ form a moiety derived from a dihydroxy compound having at least two hydroxy groups separated by at least two connecting atoms in a chain or ring, said chain or ring comprising carbon atoms, and optionally, a heteroatom or heteroatoms which can be N, S, or O; and
A is zero.

In one embodiment, for CDP-proteasome inhibitor conjugates described in any one of first to fifteenth embodiments, RB(OH)₂ or its analog is represented by formula

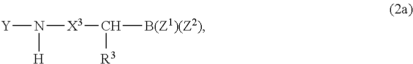

or a pharmaceutically acceptable salts thereof, wherein:

Y is one of R⁸—C(O)—, R⁸—SO₂—, R⁸—NH—C(O)— or R⁸—O—C(O)—, where R⁸ is one of alkyl, aryl, alkaryl, aralkyl, any of which can be optionally substituted, or when Y is R⁸—C(O)— or R⁸—SO₂—, then R⁸ can also be an optionally substituted 5-10 membered, saturated, partially unsaturated or aromatic heterocycle;

X³ is a covalent bond or —C(O)—CH₂—;
R³ is one of hydrogen, alkyl, cycloalkyl, aryl, a 5-10 membered saturated, partially unsaturated or aromatic heterocycle or —CH₂—R⁵, where the ring portion of any of said aryl, aralkyl, alkaryl or heterocycle can be optionally substituted;
R⁵, in each instance, is one of aryl, aralkyl, alkaryl, cycloalkyl, a 5-10 membered saturated, partially unsaturated or aromatic heterocycle or —W—R⁶, where W is a chalcogen and R⁶ is alkyl, where the ring portion of any of said aryl, aralkyl, alkaryl or heterocycle can be optionally substituted; and
Z¹ and Z² are independently alkyl, hydroxy, alkoxy, aryloxy, or together form a moiety derived from dihydroxy compound having at least two hydroxy groups separated by at least two connecting atoms in a chain or ring, said chain or ring comprising carbon atoms, and optionally, a heteroatom or heteroatoms which can be N, S, or O;
provided that when Y is R⁸—C(O)—, R⁸ is other than phenyl, benzyl or C₁-C₃ alkyl.

Alternatively, the group Y in formula (2a) above, can be:

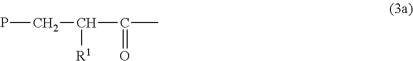

P is one of R⁷—C(O)—, R⁷—SO₂—, R⁷—NH—C(O)— or R⁷—O—C(O)—;

R⁷ is one of alkyl, aryl, alkaryl, aralkyl, any of which can be optionally substituted, or when Y is R⁷—C(O)— or R⁷—SO₂—, R⁷ can also be an optionally substituted 5-10 membered saturated, partially unsaturated or aromatic heterocycle; and
R¹ is defined above as for formula (1a).

In one embodiments, compounds of formula (1a) or (2a) described above are compounds depicted in Table 1.

TABLE 1
Inhibition of the 20S Proteasome by Boronic Ester and Acid Compounds
P—AA¹—AA²—AA³—B(Z¹)(Z²)

| Com- | | | | | |
|---|---|---|---|---|---|
| pound | P^{a} | AA¹ | AA^{2b} | AA^{3c} | Z¹, Z² |
| MG-261 | Cbz | L-Leu | L-Leu | L-Leu | pinane diol |
| MG-262 | Cbz | L-Leu | L-Leu | L-Leu | (OH)₂ |
| MG-264 | Cbz | — | L-Leu | L-Leu | pinane diol |
| MG-267 | Cbz | — | L-Nal | L-Leu | pinane diol |
| MG-268 | Cbz(N—Me) | — | L-Leu | L-Leu | (OH)₂ |
| MG-270 | Cbz | — | L-Nal | L-Leu | (OH)₂ |
| MG-272 | Cbz | — | D-(2-Nal) | L-Leu | (OH)₂ |
| MG-273 | Morph | — | L-Nal | L-Leu | (OH)₂ |
| MG-274 | Cbz | — | L-Leu | L-Leu | (OH)₂ |
| MG-278 | Morph | L-Leu | L-Leu | L-Leu | (OH)₂ |
| MG-282 | Cbz | — | L-His | L-Leu | (OH)₂ |
| MG-283 | Ac | L-Leu | L-Leu | L-Leu | (OH)₂ |
| MG-284 | 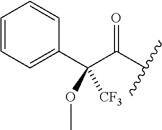 | — | — | L-Leu | (OH)₂ |
| MG-285 | Morph | — | L-Trp | L-Leu | (OH)₂ |
| MG-286 | Morph | — | L-Nal | L-Leu | diethanol- |
| | | | | | amine |
| MG-287 | Ac | — | L-Nal | L-Leu | (OH)₂ |
| MG-288 | Morph | — | L-Nal | D-Leu | (OH)₂ |
| MG-289 | Ms | — | L-(3-Pal) | L-Leu | (OH)₂ |
| MG-290 | Ac | — | L-(3-Pal) | L-Leu | (OH)₂ |
| MG-291 | Ms | — | L-Nal | L-Leu | diethanol- |
| | | | | | amine |
| MG-292 | Morph | — | 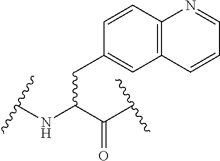 | L-Leu | (OH)₂ |
| MG-293 | Morph | — | D-Nal | D-Leu | (OH)₂ |
| MG-294 | H | — | L-(3-Pal) | L-Leu | (OH)₂ |
| MG-295 | Ms | — | L-Trp | L-Leu | (OH)₂ |
| MG-296 | (8-Quin)-SO₂ | — | L-Nal | L-Leu | (OH)₂ |
| MG-297 | Ts | — | L-Nal | L-Leu | (OH)₂ |
| MG-298 | (2-Quin)-C(O) | — | L-Nal | L-Leu | (OH)₂ |
| MG-299 | (2-quinoxalinyl)-C(O) | — | L-Nal | L-Leu | (OH)₂ |
| MG-300 | Morph | — | L-(3-Pal) | L-Leu | (OH)₂ |
| MG-301 | Ac | — | L-Trg | L-Leu | (OH)₂ |
| MG-302 | H | — | L-Nal | L-Leu | (OH)₂ |
| MG-303 | H•HCl | — | L-Nal | L-Leu | (OH)₂ |
| MG-304 | Ac | L-Leu | L-Nal | L-Leu | (OH)₂ |
| MG-305 | Morph | — | D-Nal | L-Leu | (OH)₂ |
| MG-306 | Morph | — | L-Tyr-(O-Benzyl) | L-Leu | (OH)₂ |
| MG-307 | Morph | — | L-Tyr | L-Leu | (OH)₂ |
| MG-308 | Morph | — | L-(2-Nal) | L-Leu | (OH)₂ |
| MG-309 | Morph | — | L-Phe | L-Leu | (OH)₂ |
| MG-310 | Ac | — | 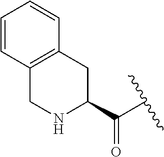 | L-Leu | (OH)₂ |
| MG-312 | Morph | — | L-(2-Pal) | L-Leu | (OH)₂ |
| MG-313 | Phenethyl-C(O) | — | — | L-Leu | (OH)₂ |
| MG-314 | (2-Quin)-C(O) | — | L-Phe | L-Leu | (OH)₂ |
| MG-315 | Morph | — | 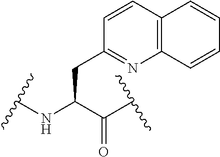 | L-Leu | (OH)₂ |
| MG-316 | H•HCl | — | 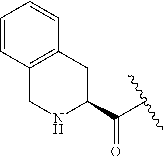 | L-Leu | (OH)₂ |
| MG-317 | Morph | — | L-Nal | L-Leu | (OH)(CH₃) |
| MG-318 | Morph | — | L-Nal | L-Leu | (CH₃)₂ |
| MG-319 | H•HCl | — | L-Pro | L-Leu | (OH)₂ |
| MG-321 | Morph | — | L-Nal | L-Phe | (OH)₂ |
| MG-322 | Morph | — | L-homoPhe | L-Leu | (OH)₂ |
| MG-323 | Ac | — | — | L-Leu | (OH)₂ |
| MG-324 | 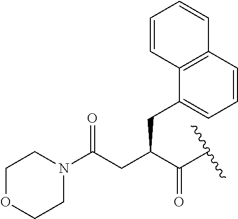 | — | — | L-Leu | H |
| MG-325 | (2-Quin)-C(O) | — | L-homoPhe | L-Leu | (OH)₂ |
| MG-328 | Bz | — | L-Nal | L-Leu | (OH)₂ |
| MG-329 | Cyclohexyl-C(O) | — | L-Nal | L-Leu | (OH)₂ |
| MG-332 | Cbz(N—Me) | — | L-Nal | L-Leu | (OH)₂ |
| MG-333 | H•HCl | — | L-Nal | L-Leu | (OH)₂ |
| MG-334 | H•HCl(N—Me) | — | L-Nal | L-Leu | (OH)₂ |
| MG-336 | (3-Pyr)—C(O) | — | L-Phe | L-Leu | (OH)₂ |
| MG-337 | H•HCl | — | 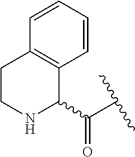 | L-Leu | (OH)₂ |
| MG-338 | (2-Quin)-C(O) | — | L-(2-Pal) | L-Leu | (OH)₂ |
| MG-339 | H•HCl | — | 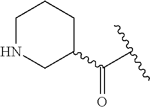 | L-Leu | (OH)₂ |
| MG-340 | H | — | 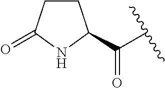 | L-Leu | (OH)₂ |
| MG-341 | (2-Pyz)—C(O) | — | L-Phe | L-Leu | (OH)₂ |
| MG-342 | Bn | — | 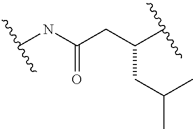 | — | (OH)₂ |
| MG-343 | (2-Pyr)—C(O) | — | L-Phe | L-Leu | (OH)₂ |
| MG-344 | Ac | — | 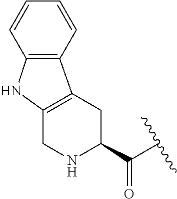 | L-Leu | (OH)₂ |
| MG-345 | Bz | — | L-(2-Pal) | L-Leu | (OH)₂ |
| MG-346 | Cyclohexyl-C(O) | — | L-(2-Pal) | L-Leu | (OH)₂ |
| MG-347 | (8-Quin)-SO₂ | — | L-(2-Pal) | L-Leu | (OH)₂ |
| MG-348 | H•HCl | — | 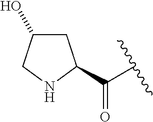 | L-Leu | (OH)₂ |
| MG-349 | H•HCl | — | 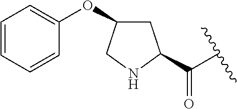 | L-Leu | (OH)₂ |
| MG-350 | 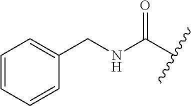 | — | L-Phe | L-Leu | (OH)₂ |
| MG-351 | H•HCl | — | L-(2-Pal) | L-Leu | (OH)₂ |
| MG-352 | Phenylethyl-C(O) | — | L-Phe | L-Leu | (OH)₂ |
| MG-353 | Bz | — | L-Phe | L-Leu | (OH)₂ |
| MG-354 | (8-Quin)-SO₂ | — | 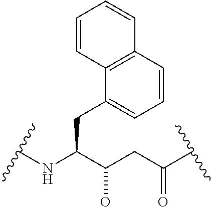 | L-Leu | (OH)₂ |
| MG-356 | Cbz | — | L-Phe | L-Leu | (OH)₂ |
| MG-357 | H•HCl | — | 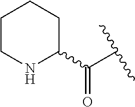 | L-Leu | (OH)₂ |
| MG-358 | (3-Furanyl)-C(O) | — | L-Phe | L-Leu | (OH)₂ |
| MG-359 | H•HCl | — | 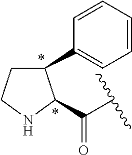 | L-Leu | (OH)₂ |
| MG-361 | (3-Pyrrolyl)-C(O) | — | L-Phe | L-Leu | (OH)2 |
| MG-362 | 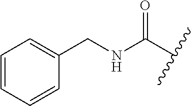 | — | — | L-Leu | (OH)₂ |
| MG-363 | H•HCl | — | 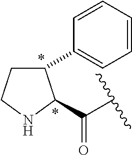 | L-Leu | (OH)₂ |
| MG-364 | Phenethyl-C(O) | — | — | L-Leu | (OH)₂ |
| MG-366 | H•HCl | — | 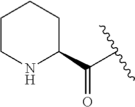 | L-Leu | (OH)₂ |
| MG-368 | (2-Pyz)—C(O) | — | L-(2-Pal) | L-Leu | (OH)₂ |
| MG-369 | H•HCl | — | 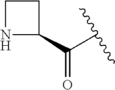 | L-Leu | (OH)₂ |
| MG-380 | (8-Quin)SO₂ | — | L-Phe | L-Leu | (OH)₂ |
| MG-382 | (2-Pyz)—C(O) | — | L-(4-F)-Phe | L-Leu | (OH)₂ |
| MG-383 | (2-Pyr)—C(O) | — | L-(4-F)-Phe | L-Leu | (OH)₂ |
| MG-385 | H•HCl | — | 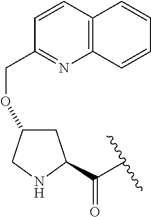 | L-Leu | (OH)₂ |
| MG-386 | H•HCl | — | 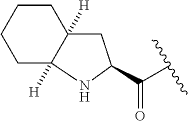 | L-Leu | (OH)₂ |
| MG-387 | Morph | — | 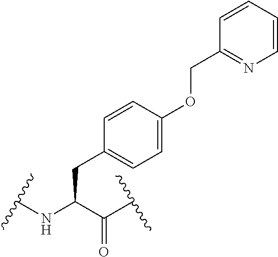 | L-Leu | (OH)₂ |

^{a}Cbz = carbobenzyloxy; MS = methylsulfonyl; Morph = 4-morpholinecarbonyl; (8-Quin)—SO₂ = 8-quinolinesulfonyl; (2-Quin)—C(O) = 2-quinolinecarbonyl; Bz = benzoyl; (2-Pyr)—C(O) = 2-pyridinecarbonyl; (3-Pyr)—C(O) = 3-pyridinecarbonyl; (2-Pyz)—C(O) = 2-pyrazinecarbonyl.
^{b}Nal = β-(1-naphthyl)alanine; (2-Nal) = β-(2-naphthyl)alanine; (2-Pal) = β-(2-pyridyl)alanine; (3-Pal) = β-(3-pyridyl)alanine; homoPhe = homophenylalanine; (4-F)-Phe = (4-flurophenyl)alanine.
^{c}B(Z¹)(Z²) takes the place of the carboxyl group of AA³.

In another embodiment, compounds of formula (1a) or (2a) described above are selected from the following compounds as well as pharmaceutically acceptable salts and boronate esters thereof:
N-(4-morpholine)carbonyl-β-(1-naphthyl)-L-alanine-L-leucine boronic acid,
N-(8-quinoline)sulfonyl-β-(1-naphthyl)-L-alanine-L-leucine boronic acid,
N-(2-pyrazine)carbonyl-L-phenylalanine-L-leucine boronic acid,
L-proline-L-leucine boronic acid,
N-(2-quinoline)carbonyl-L-homophenylalanine-L-leucine boronic acid,
N-(3-pyridine)carbonyl-L-phenylalanine-L-leucine boronic acid,
N-(3-phenylpropionyl)-L-phenylalanine-L-leucine boronic acid,
N-(4-morpholine)carbonyl-L-phenylalanine-L-leucine boronic acid,
N-(4-morpholine)carbonyl-(O-benzyl)-L-tyrosine-L-leucine boronic acid,
N-(4-morpholine)carbonyl-L-tyrosine-L-leucine boronic acid, and
N-(4-morpholine)carbonyl-[O-(2-pyridylmethyl)]-L-tyrosine-L-leucine boronic acid.

In one embodiment, for the CDP-proteasome inhibitor conjugates described in any one of first to fifteenth embodiments, RB(OH)₂ or RB(Y)₂ is represented by the formula (3a):

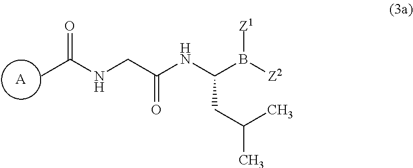

or a pharmaceutically acceptable salt or boronic acid anhydride thereof, wherein:

Z¹ and Z² are each independently hydroxy, alkoxy, aryloxy, or aralkoxy; or Z¹ and Z² together form a moiety derived from a boronic acid completing agent; and

Ring A is selected from the group consisting of:

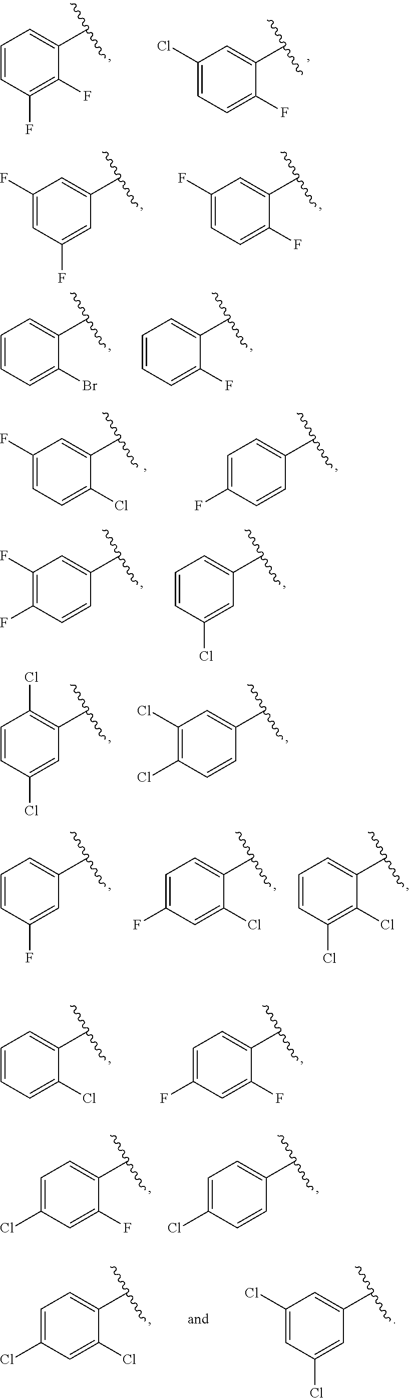

More specifically, compounds of formula (3a) are referred to by the following chemical names:
I-1 [(1R)-1-({[(2,3-difluorobenzoyl)amino]acetyl}amino)-3-methylbutyl]boronic acid
I-2 [(1R)-1-({[(5-chloro-2-fluorobenzoyl)amino]acetyl}amino)-3-methylbutyl]boronic acid
I-3 [(1R)-1-({[(3,5-difluorobenzoyl)amino]acetyl}amino)-3-methylbutyl]boronic acid
I-4 [(1R)-1-({[(2,5-difluorobenzoyl)amino]acetyl}amino)-3-methylbutyl]boronic acid
I-5 [(1R)-1-({[(2-bromobenzoyl)amino]acetyl}amino)-3-methylbutyl]boronic acid
I-6 [(1R)-1-({[(2-fluorobenzoyl)amino]acetyl}amino)-3-methylbutyl]boronic acid
I-7 [(1R)-1-({[(2-chloro-5-fluorobenzoyl)amino]acetyl}amino)-3-methylbutyl]boronic acid
I-8 [(1R)-1-({[(4-fluorobenzoyl)amino]acetyl}amino)-3-methylbutyl]boronic acid
I-9 [(1R)-1-({[(3,4-difluorobenzoyl)amino]acetyl}amino)-3-methylbutyl]boronic acid
I-10 [(1R)-1-({[(3-chlorobenzoyl)amino]acetyl}amino)-3-methylbutyl]boronic acid
I-11 [(1R)-1-({[(2,5-dichlorobenzoyl)amino]acetyl}amino)-3-methylbutyl]boronic acid
I-12 [(1R)-1-({[(3,4-dichlorobenzoyl)amino]acetyl}amino)-3-methylbutyl]boronic acid
I-13 [(1R)-1-({[(3-fluorobenzoyl)amino]acetyl}amino)-3-methylbutyl]boronic acid
I-14 [(1R)-1-({[(2-chloro-4-fluorobenzoyl)amino]acetyl}amino)-3-methylbutyl]boronic acid
I-15 [(1R)-1-({[(2,3-dichlorobenzoyl)amino]acetyl}amino)-3-methylbutyl]boronic acid
I-16 [(1R)-1-({[(2-chlorobenzoyl)amino]acetyl}amino)-3-methylbutyl]boronic acid
I-17 [(1R)-1-({[(2,4-difluorobenzoyl)amino]acetyl}amino)-3-methylbutyl]boronic acid
I-18 [(1R)-1-({[(4-chloro-2-fluorobenzoyl)amino]acetyl}amino)-3-methylbutyl]boronic acid
I-19 [(1R)-1-({[(4-chlorobenzoyl)amino]acetyl}amino)-3-methylbutyl]boronic acid
I-20 [(1R)-1-({[(2,4-dichlorobenzoyl)amino]acetyl}amino)-3-methylbutyl]boronic acid
I-21 [(1R)-1-({[(3,5-dichlorobenzoyl)amino]acetyl}amino)-3-methylbutyl]boronic acid.

In another embodiment, for the CDP-proteasome inhibitor conjugates described in any one of first to fifteenth embodiments, RB(OH)₂ or RB(Y)₂ is represented by formula (4a):

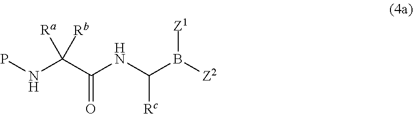

or a pharmaceutically acceptable salt or boronic acid anhydride thereof, wherein:

P is hydrogen or an amino-group-blocking moiety;

R^{a} is a C₁₋₄ aliphatic or C₁₋₄ fluoroaliphatic group that is substituted with 0-1 R^{A}; or R^{a} and R^{b} taken together with the carbon atom to which they are attached, form a substituted or unsubstituted 3- to 6-membered cycloaliphatic group;

R^{A} is a substituted or unsubstituted aromatic or cycloaliphatic ring;

R^{b} is a C₁₋₄ aliphatic or C₁₋₄ fluoroaliphatic group; or R^{a} and R^{b}, taken together with the carbon atom to which they are attached, form a substituted or unsubstituted 3- to 6-membered cycloaliphatic group;

R^{c} is a C₁₋₄ aliphatic or C₁₋₄ fluoroaliphatic group that is substituted with 0-1 R^{C}; R^{C} is a substituted or unsubstituted aromatic or cycloaliphatic ring; and

Z¹ and Z² are each independently hydroxy, alkoxy, aryloxy, or aralkoxy; or Z¹ and Z² together form a moiety derived from a boronic acid complexing agent.

Representative examples of compounds of formula (4a), wherein Z¹ and Z² are each —OH are shown as the following:

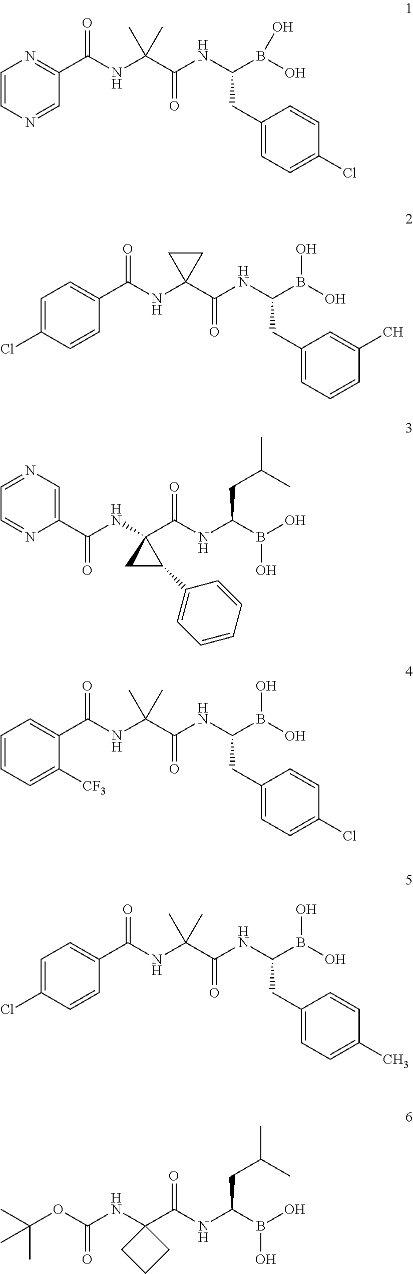

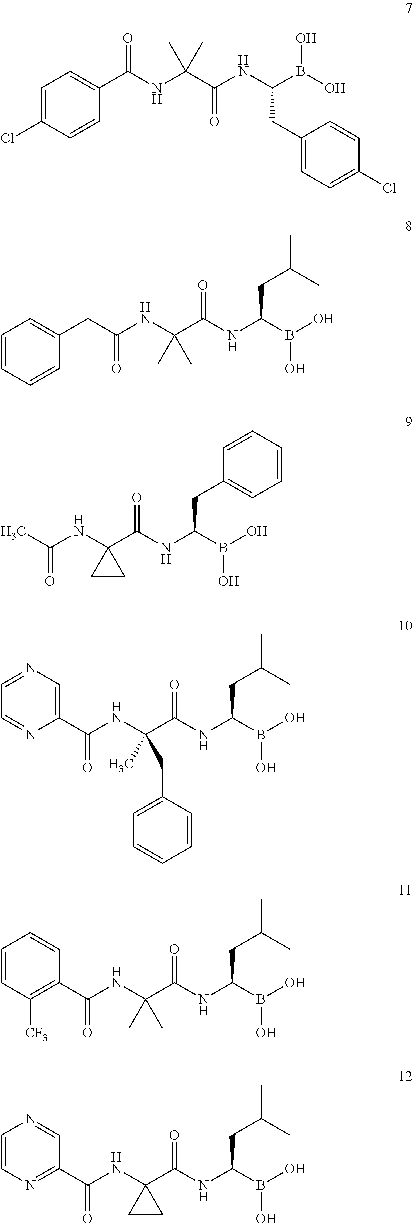

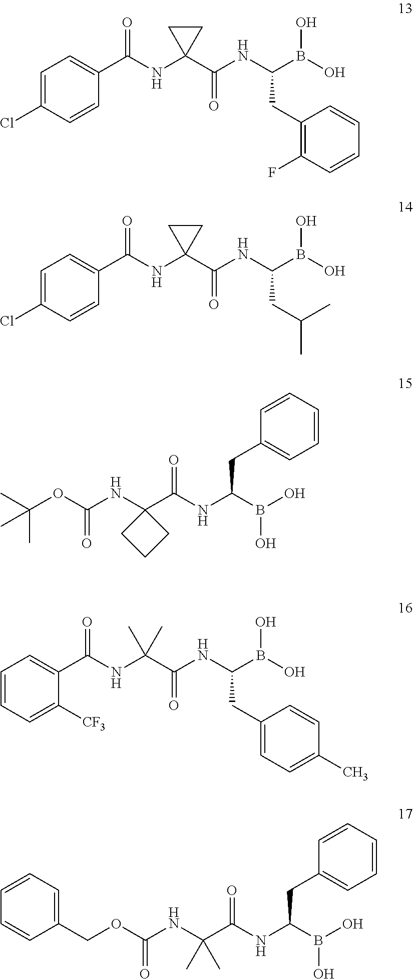

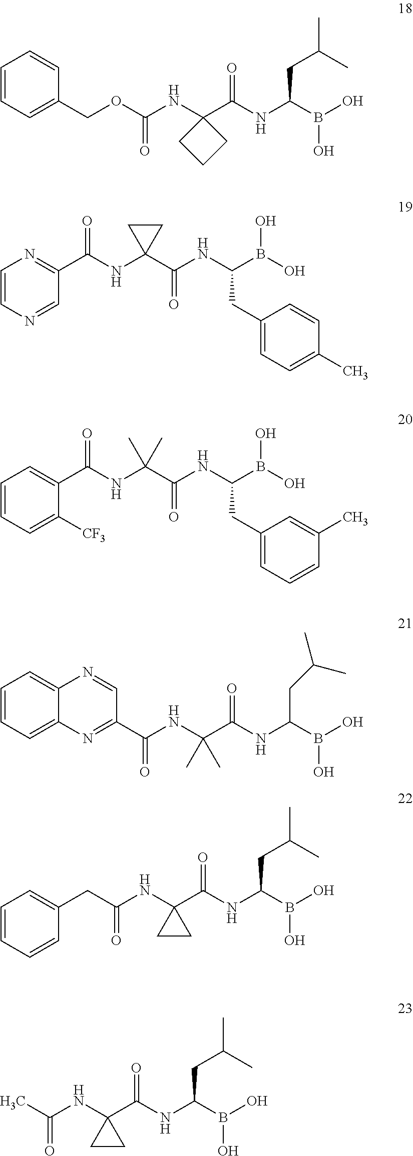

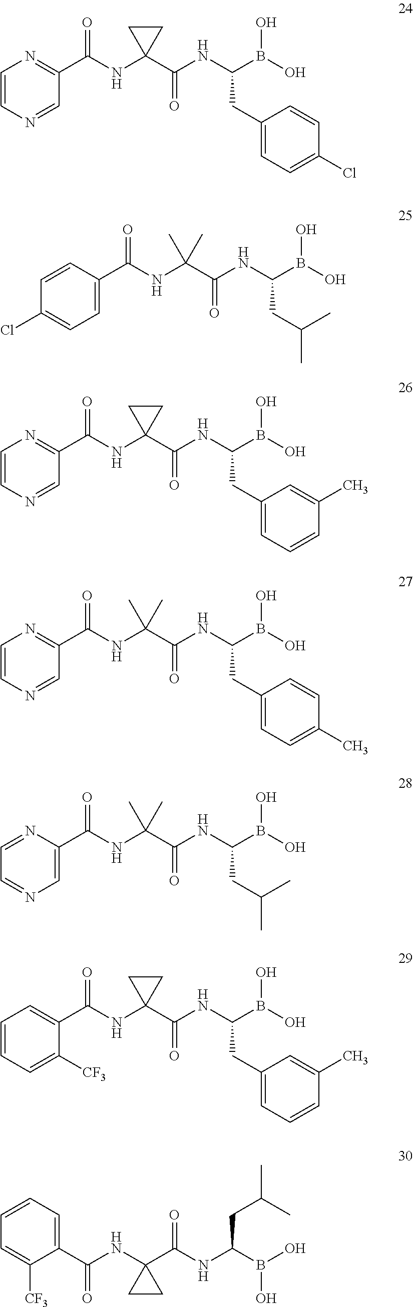

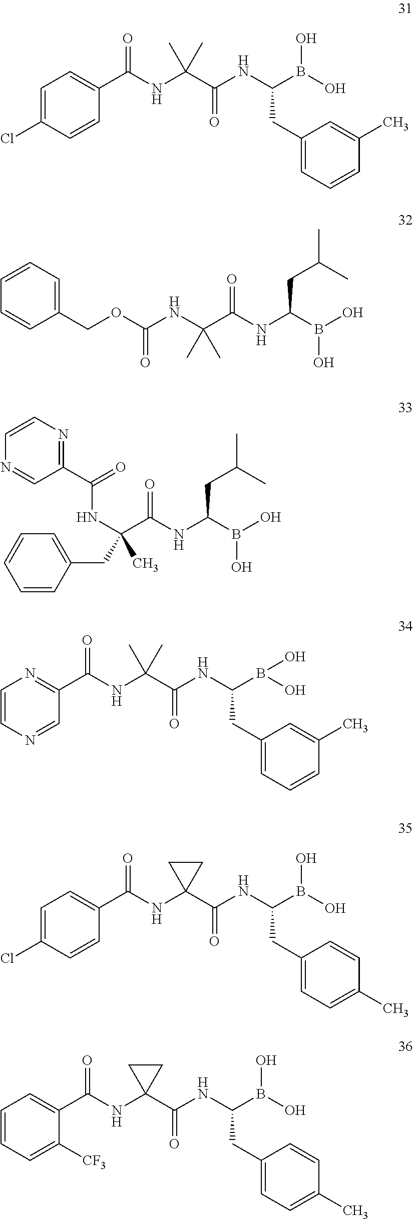

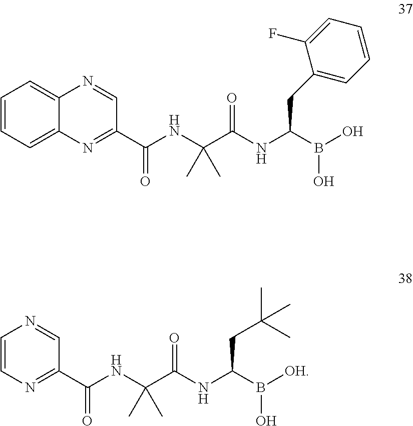

CDP-proteasome inhibitor (such as a boronic acid containing proteasome inhibitor, e.g., bortezomib) conjugates can be made using many different combinations of components described herein. For example, various combinations of cyclodextrins (e.g., beta-cyclodextrin), comonomers (e.g., PEG containing comonomers), linkers linking the cyclodextrins and comonomers, and/or linkers tethering the proteasome inhibitor (such as a boronic acid containing proteasome inhibitor, e.g., bortezomib) to the CDP are described herein.

Table 2 is a table depicting examples of different CDP-proteasome inhibitor conjugates. The CDP-proteasome inhibitor conjugates in Table 2 are represented by the following formula:

CDP-CO-L-D

In this formula,

CDP is the cyclodextrin-containing polymer shown below:

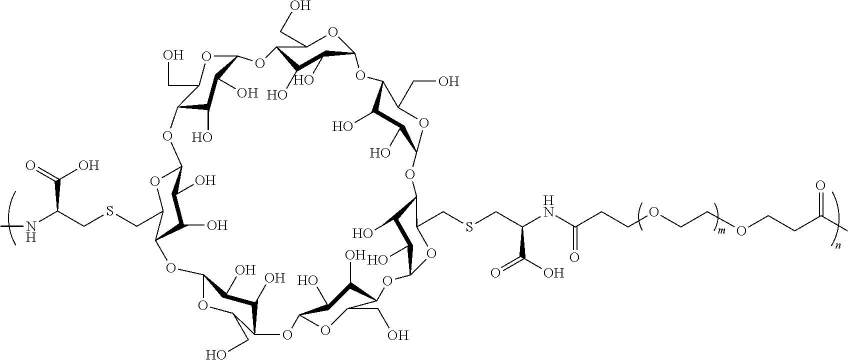

wherein the group

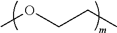

has a Mw of 3.4 kDa or less and n is at least 4, 5, 6, 7, 8, 9, 10, 11, 12, 13, 14, 15, 16, 17, 18, 19 or 20;

D is —B—R, wherein R is the non-boronic acid moiety in bortezomib;

CO represents the carbonyl group of the cysteine residue of the CDP; and

L represents a linker group between the CDP and the boronic acid.

Note that the proteasome inhibitor (such as a boronic acid containing proteasome inhibitor, e.g., bortezomib) is conjugated to the CDP through the carboxylic acid moieties of the polymer as provided above. Full loading of the proteasome inhibitor (such as a boronic acid containing proteasome inhibitor, e.g., bortezomib) onto the CDP is not required. In some embodiments, at least one, e.g., at least 2, 3, 4, 5, 6 or 7, of the carboxylic acid moieties remains unreacted with the proteasome inhibitor (such as a boronic acid containing proteasome inhibitor, e.g., bortezomib) after conjugation (e.g., a plurality of the carboxylic acid moieties remain unreacted);

L has a terminal amino group that is bonded to the cysteine acid carbonyl of CDP. The other terminal of L comprises two functional groups that bind to the boron atom in bortezomib and upon binding to bortezomib, the two functional groups displace the two —OH groups in bortezomib that are bonded to the boron atom.

As provided in Table 2, the column with the heading “Boronic Acid” indicates which pharmaceutically active agent, preferably a proteasome inhibitor, comprising a boronic acid that is included in the CDP-proteasome inhibitor conjugate.

The two columns on the right of Table 2 indicate respectively, the process for producing the CDP-proteasome inhibitor conjugate, and the final product of the process for producing the CDP-proteasome inhibitor conjugate.

The processes referred to in Table 2 are given a letter representation, e.g., Process A and Process B, as seen in the second column from the right. The steps for each these processes respectively are provided below.

Process A: Couple the optionally protected L to CDP; deprotect L-CDP if protected; and conjugate the boronic acid.

Process B: Conjugate the optionally protected L to boronic acid; deprotect L-boronic acid; and couple L-boronic acid to CDP.

As shown specifically in Table 2, the CDP-proteasome inhibitor conjugates can be prepared using a variety of methods known in the art, including those described herein.

For Table 2, the structures for the CDP-proteasome inhibitor are represented by CDP-L-boronic acid, wherein Z¹ and Z² each represent bonds to the boron atom of the conjugated drug. For example, the CDP-bortezomib conjugate is represented by CDP-L-B-(L)-CH(CH₂CH(CH₃)₂)NH-(L)-Phe-CO-pyrazine.

TABLE 2

| | | | Process for | |
|---|---|---|---|---|
| | | | Preparation of | |
| | | | boronic acid linker | |
| Example | | Boronic Acid | CDP molecules | Final Product |
| | —NH(CH₂)₆C(CH₃)(OZ¹)C(CH₃)₂OZ² | bortezomib | Process A | bortezomib |
| | | | | linked to CDP |
| | —NH(CH₂)₄CH(CH₂OZ¹)CH₂OZ² | bortezomib | Process A | bortezomib |
| | | | | linked to CDP |
| | —NH(CH₂)₆C(CH₃)(OZ¹)CH₂NH(CH₃)Z² | bortezomib | Process A | bortezomib |
| | | | | linked to CDP |
| | —NH(CH₂)₆N(CH₂CH₂OZ¹)CH₂CH₂OZ² | bortezomib | Process A | bortezomib |
| | | | | linked to CDP |
| | —NH(CH₂)₆N(CH₂CO₂Z¹)CH₂CO₂Z² | bortezomib | Process A | bortezomib |
| | | | | linked to CDP |
| | —NH(CH₂)₆N((CH₂)₂OZ¹)(CH₂)₂N(CH₃)Z² | bortezomib | Process A | bortezomib |
| | | | | linked to CDP |
| | —NH(CH₂)₆C(CH₃)(OZ¹)C(CH₃)₂OZ² | bortezomib | Process B | bortezomib |
| | | | | linked to CDP |
| | —NH(CH₂)₄CH(CH₂OZ¹)CH₂OZ² | bortezomib | Process B | bortezomib |
| | | | | linked to CDP |
| | —NH(CH₂)₆C(CH₃)(OZ¹)CH₂NH(CH₃)Z² | bortezomib | Process B | bortezomib |
| | | | | linked to CDP |
| | —NH(CH₂)₆N(CH₂CH₂OZ¹)CH₂CH₂OZ² | bortezomib | Process B | bortezomib |
| | | | | linked to CDP |
| | —NH(CH₂)₆N(CH₂CO₂Z¹)CH₂CO₂Z² | bortezomib | Process B | bortezomib |
| | | | | linked to CDP |
| | —NH(CH₂)₆N((CH₂)₂OZ¹)(CH₂)₂N(CH₃)Z² | bortezomib | Process B | bortezomib |
| | | | | linked to CDP |
| | —NHCH₂CH₂OCH₂CH₂C(CH₃)(OZ¹)C(CH₃)₂OZ² | bortezomib | Process A | bortezomib |
| | | | | linked to CDP |
| | —NHCH₂CH₂OCH₂CH₂CH(CH₂OZ¹)CH₂OZ² | bortezomib | Process A | bortezomib |
| | | | | linked to CDP |
| | —NHCH₂CH₂OCH₂CH₂C(CH₃)(OZ¹)CH₂NH(CH₃)Z² | bortezomib | Process A | bortezomib |
| | | | | linked to CDP |
| | —NHCH₂CH₂OCH₂CH₂N(CH₂CH₂OZ¹)CH₂CH₂OZ² | bortezomib | Process A | bortezomib |
| | | | | linked to CDP |
| | —NHCH₂CH₂OCH₂CH₂N(CH₂CO₂Z¹)CH₂CO₂Z² | bortezomib | Process A | bortezomib |
| | | | | linked to CDP |
| | —NHCH₂CH₂OCH₂CH₂N((CH₂)₂OZ¹)(CH₂)₂N(CH₃)Z² | bortezomib | Process A | bortezomib |
| | | | | linked to CDP |
| | —NHCH₂CH₂OCH₂CH₂C(CH₃)(OZ¹)C(CH₃)₂OZ² | bortezomib | Process B | bortezomib |
| | | | | linked to CDP |
| | —NHCH₂CH₂OCH₂CH₂CH(CH₂OZ¹)CH₂OZ² | bortezomib | Process B | bortezomib |
| | | | | linked to CDP |
| | —NHCH₂CH₂OCH₂CH₂C(CH₃)(OZ¹)CH₂NH(CH₃)Z² | bortezomib | Process B | bortezomib |
| | | | | linked to CDP |
| | —NHCH₂CH₂OCH₂CH₂N(CH₂CH₂OZ¹)CH₂CH₂OZ² | bortezomib | Process B | bortezomib |
| | | | | linked to CDP |
| | —NHCH₂CH₂OCH₂CH₂N(CH₂CO₂Z¹)CH₂CO₂Z² | bortezomib | Process B | bortezomib |
| | | | | linked to CDP |
| | —NHCH₂CH₂OCH₂CH₂N((CH₂)₂OZ¹)(CH₂)₂N(CH₃)Z² | bortezomib | Process B | bortezomib |
| | | | | linked to CDP |
| | —NHCH₂COOCH₂CH₂C(CH₃)(OZ¹)C(CH₃)₂OZ² | bortezomib | Process A | bortezomib |
| | | | | linked to CDP |
| | —NHCH₂COOCH₂CH₂CH(CH₂OZ¹)CH₂OZ² | bortezomib | Process A | bortezomib |
| | | | | linked to CDP |
| | —NHCH₂COOCH₂CH₂C(CH₃)(OZ¹)CH₂NH(CH₃)Z² | bortezomib | Process A | bortezomib |
| | | | | linked to CDP |
| | —NHCH₂COOCH₂CH₂N(CH₂CH₂OZ¹)CH₂CH₂OZ² | bortezomib | Process A | bortezomib |
| | | | | linked to CDP |
| | —NHCH₂COOCH₂CH₂N(CH₂CO₂Z¹)CH₂CO₂Z² | bortezomib | Process A | bortezomib |
| | | | | linked to CDP |
| | —NHCH₂COOCH₂CH₂N((CH₂)₂OZ¹)(CH₂)₂N(CH₃)Z² | bortezomib | Process A | bortezomib |
| | | | | linked to CDP |
| | —NHCH₂COOCH₂CH₂C(CH₃)(OZ¹)C(CH₃)₂OZ² | bortezomib | Process B | bortezomib |
| | | | | linked to CDP |
| | —NHCH₂COOCH₂CH₂CH(CH₂OZ¹)CH₂OZ² | bortezomib | Process B | bortezomib |
| | | | | linked to CDP |
| | —NHCH₂COOCH₂CH₂C(CH₃)(OZ¹)CH₂NH(CH₃)Z² | bortezomib | Process B | bortezomib |
| | | | | linked to CDP |
| | —NHCH₂COOCH₂CH₂N(CH₂CH₂OZ¹)CH₂CH₂OZ² | bortezomib | Process B | bortezomib |
| | | | | linked to CDP |
| | —NHCH₂COOCH₂CH₂N(CH₂CO₂Z¹)CH₂CO₂Z² | bortezomib | Process B | bortezomib |
| | | | | linked to CDP |
| | —NHCH₂COOCH₂CH₂N((CH₂)₂OZ¹)(CH₂)₂N(CH₃)Z² | bortezomib | Process B | bortezomib |
| | | | | linked to CDP |
| | —NHZCONHCH₂CH₂C(CH₃)(OZ¹)C(CH₃)₂OZ² | bortezomib | Process A | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂CH(CH₂OZ¹)CH₂OZ² | bortezomib | Process A | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂C(CH₃)(OZ¹)CH₂NH(CH₃)Z² | bortezomib | Process A | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂N(CH₂CH₂OZ¹)CH₂CH₂OZ² | bortezomib | Process A | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂N(CH₂CO₂Z¹)CH₂CO₂Z² | bortezomib | Process A | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂N((CH₂)₂OZ¹)(CH₂)₂N(CH₃)Z² | bortezomib | Process A | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂C(CH₃)(OZ¹)C(CH₃)₂OZ² | bortezomib | Process B | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂CH(CH₂OZ¹)CH₂OZ² | bortezomib | Process B | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂C(CH₃)(OZ¹)CH₂NH(CH₃)Z² | bortezomib | Process B | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂N(CH₂CH₂OZ¹)CH₂CH₂OZ² | bortezomib | Process B | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂N(CH₂CO₂Z¹)CH₂CO₂Z² | bortezomib | Process B | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |
| | —NHZCONHCH₂CH₂N((CH₂)₂OZ¹)(CH₂)₂N(CH₃)Z² | bortezomib | Process B | bortezomib |
| | Z is a mono, di, or tripeptide or other peptide | | | linked to CDP |
| | or derivative thereof where NH and CO | | | |
| | represent the amino and acid terminus of the | | | |
| | amino acid or peptide | | | |

One or more protecting groups can be used in the processes described above to make the CDP-proteasome inhibitor conjugates described herein. In some embodiments, the protecting group is removed and, in other embodiments, the protecting group is not removed. If a protecting group is not removed, then it can be selected so that it is removed in vivo (e.g., acting as a prodrug). An example is hexanoic acid which has been shown to be removed by lipases in vivo if used to protect a hydroxyl group in doxorubicin. Protecting groups are generally selected for both the reactive groups of the proteasome inhibitor and the reactive groups of the linker that are not targeted to be part of the coupling reaction. The protecting group should be removable under conditions which will not degrade the proteasome inhibitor and/or linker material. Examples include t-butyldimethylsilyl (“TBDMS”), TROC (derived from 2,2,2-trichloroethoxy chloroformate), carboxybenzyl (“CBz”) and tert-butyloxycarbonyl (“Boc”). Carboxybenzyl (“CBz”) can also be used in place of TROC if there is selectivity seen for removal over olefin reduction. This can be addressed by using a group which is more readily removed by hydrogenation such as -methoxybenzyl OCO—. Other protecting groups may also be acceptable. One of skill in the art can select suitable protecting groups for the products and methods described herein.

### CDP-Proteasome Inhibitor Conjugate Characteristics

In some embodiments, the CDP and/or CDP-proteasome inhibitor (such as a boronic acid containing proteasome inhibitor) conjugates, e.g., CDP-bortezomib conjugate, as described herein have polydispersities less than about 3, or even less than about 2.

One embodiment of the present invention provides an improved delivery of certain proteasome inhibitor (such as a boronic acid containing proteasome inhibitor, e.g., bortezomib) by covalently conjugating them to a CDP. Such conjugation improves the aqueous solubility and hence the bioavailability of the proteasome inhibitor (such as a boronic acid containing proteasome inhibitor, e.g., bortezomib). Accordingly, in one embodiment of the invention, the proteasome inhibitor is a hydrophobic compound with a log P>0.4, >0.6, >0.8, >1, >2, >3, >4, or even >5. In other embodiments, a proteasome inhibitor (such as a boronic acid containing proteasome inhibitor, e.g., bortezomib) may be attached to another compound, such as an amino acid, prior to covalently attaching the conjugate onto the CDP.

The CDP-proteasome inhibitor (such as a boronic acid containing proteasome inhibitor) conjugates, e.g., CDP-bortezomib conjugates, described herein preferably have molecular weights in the range of 10,000 to 500,000; 30,000 to 200,000; or even 70,000 to 150,000 amu. In certain embodiments as disclosed herein, the compound has a number average (Mₙ) molecular weight between 1,000 to 500,000 amu, or between 5,000 to 200,000 amu, or between 10,000 to 100,000 amu. One method to determine molecular weight is by gel permeation chromatography (“GPC”), e.g., mixed bed columns, CH₂Cl₂ solvent, light scattering detector, and off-line dn/dc. Other methods are known in the art.

In certain embodiments as disclosed herein, the CDP-proteasome inhibitor (such as a boronic acid containing proteasome inhibitor) conjugate, e.g., a CDP-bortezomib conjugate, is biodegradable or bioerodable.

In certain embodiments as disclosed herein, the proteasome inhibitor (such as a boronic acid containing proteasome inhibitor, e.g., bortezomib) or prodrug thereof makes up at least 3% (e.g., at least about 5%) by weight of the compound. In certain embodiments, the therapeutic agent or prodrug thereof makes up at least 20% by weight of the compound. In certain embodiments, the therapeutic agent or prodrug thereof makes up at least 5%, 10%, 15%, or at least 20% by weight of the compound.

In other embodiments, the CDP-proteasome inhibitor (such as a boronic acid containing proteasome inhibitor) conjugate, e.g., a CDP-bortezomib conjugate, may be a flexible or flowable material. When the CDP used is itself flowable, the CDP composition of the invention, even when viscous, need not include a biocompatible solvent to be flowable, although trace or residual amounts of biocompatible solvents may still be present.

When a solvent is used to facilitate mixing or to maintain the flowability of the CDP-proteasome inhibitor (such as a boronic acid containing proteasome inhibitor) conjugate, e.g., a CDP-bortezomib conjugate, it should be non-toxic, otherwise biocompatible, and should be used in relatively small amounts. Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethylsulfoxide, tetrahydrofuran, caprolactam, oleic acid, or 1-dodecylazacylcoheptanone. Preferred solvents include N-methylpyrrolidone, 2-pyrrolidone, dimethylsulfoxide, and acetone because of their solvating ability and their biocompatibility.

In certain embodiments, the CDP-proteasome inhibitor (such as a boronic acid containing proteasome inhibitor) conjugate, e.g., a CDP-bortezomib conjugate, are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methylpyrrolidone, dimethylformamide, and dimethylsulfoxide.

In certain embodiments, the CDP-proteasome inhibitor (such as a boronic acid containing proteasome inhibitor) conjugate, e.g., a CDP-bortezomib conjugate, described herein, upon contact with body fluids, undergo gradual degradation. The life of a biodegradable polymer in vivo depends upon, among other things, its molecular weight, crystallinity, biostability, and the degree of crosslinking In general, the greater the molecular weight, the higher the degree of crystallinity, and the greater the biostability, the slower biodegradation will be.

If a subject composition is formulated with a proteasome inhibitor (such as a boronic acid containing proteasome inhibitor, e.g., bortezomib) or other material, release of the proteasome inhibitor (such as a boronic acid containing proteasome inhibitor, e.g., bortezomib) or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the proteasome inhibitor (such as a boronic acid containing proteasome inhibitor, e.g., bortezomib) or any other material associated with the polymer.

A variety of factors may affect the desired rate of hydrolysis of CDP-proteasome inhibitor (such as a boronic acid containing proteasome inhibitor) conjugates, e.g., a CDP-bortezomib conjugate, the desired softness and flexibility of the resulting solid matrix, rate and extent of bioactive material release. Some of such factors include the selection/identity of the various subunits, the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted.

One protocol generally accepted in the field that may be used to determine the release rate of a therapeutic agent such as a proteasome inhibitor (such as a boronic acid containing proteasome inhibitor, e.g., bortezomib) or other material loaded in the CDP-proteasome inhibitor (such as a boronic acid containing proteasome inhibitor, e.g., bortezomib) conjugates of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different CDP-proteasome (such as a boronic acid containing proteasome inhibitor, e.g., bortezomib) conjugates of the present invention may be compared by subjecting them to such a protocol. In certain instances, it may be necessary to process polymeric systems in the same fashion to allow direct and relatively accurate comparisons of different systems to be made. For example, the present invention teaches several different methods of formulating the CDP-proteasome inhibitor (such as a boronic acid containing proteasome inhibitor) conjugates, e.g., a CDP-bortezomib conjugate. Such comparisons may indicate that any one CDP-proteasome inhibitor (such as a boronic acid containing proteasome inhibitor) conjugate, e.g., a CDP-bortezomib conjugate, releases incorporated material at a rate from about 2 or less to about 1000 or more times faster than another polymeric system.

Alternatively, a comparison may reveal a rate difference of about 3, 5, 7, 10, 25, 50, 100, 250, 500 or 750 times. Even higher rate differences are contemplated by the present invention and release rate protocols.

In certain embodiments, when formulated in a certain manner, the release rate for CDP-proteasome inhibitor (such as a boronic acid containing proteasome inhibitor) conjugates, e.g., a CDP-bortezomib conjugate, of the present invention may present as mono- or bi-phasic.

Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 500 or more ng/day/mg. Alternatively, the release rate may be about 0.05, 0.5, 5, 10, 25, 50, 75, 100, 125, 150, 175, 200, 250, 300, 350, 400, 450, or 500 ng/day/mg. In still other embodiments, the release rate of any incorporated material may be 10,000 ng/day/mg, or even higher. In certain instances, materials incorporated and characterized by such release rate protocols may include therapeutic agents, fillers, and other substances.

In another aspect, the rate of release of any material from any CDP-proteasome inhibitor (such as a boronic acid containing proteasome inhibitor) conjugate, e.g., a CDP-bortezomib conjugate, of the present invention may be presented as the half-life of such material in the matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

### Physical Structures of the CDP-proteasome inhibitor conjugates

The CDP-proteasome inhibitor (such as a boronic acid containing proteasome inhibitor) conjugates, e.g., a CDP-bortezomib conjugate, may be formed in a variety of shapes. For example, in certain embodiments, the CDP-proteasome inhibitor (such as a boronic acid containing proteasome inhibitor) conjugates, e.g., a CDP-bortezomib conjugate, may be presented in the form of a nanoparticle. In one embodiment, the CDP-proteasome inhibitor (such as a boronic acid containing proteasome inhibitor) conjugate, e.g., a CDP-bortezomib conjugate, self assembles into a nanoparticle. In one embodiment, the CDP-proteasome inhibitor (such as a boronic acid containing proteasome inhibitor) conjugate, e.g., a CDP-bortezomib conjugate, self assembles into a nanoparticle in an aqueous solution, e.g., water.

In addition to intracellular delivery of a proteasome inhibitor (such as a boronic acid containing proteasome inhibitor, e.g., bortezomib), it also possible that nanoparticles of the CDP-proteasome inhibitor (such as a boronic acid containing proteasome inhibitor) conjugate, e.g., a CDP-bortezomib conjugate, may undergo endocytosis, thereby obtaining access to the cell. The frequency of such an endocytosis process will likely depend on the size of any nanoparticle.

In one embodiment, the surface charge of the molecule is neutral, or slightly negative. In some embodiments, the zeta potential of the particle surface is from about −80 mV to about 50 mV.

### Conjugate Number

Conjugate number, as used herein, is the number of cyclodextrin containing polymer (“CDP”) therapeutic agent conjugate molecules, present in a particle or nanoparticle. For purposes of determining conjugate number, a particle or nanoparticle is an entity having one, or typically, more than one CDP-proteasome inhibitor conjugate molecules, which, at the concentration suitable for administration to humans, behaves as a single unit in any of water, e.g., water at neutral pH, PBS, e.g., PBS at pH 7.4, or in a formulation in which it will be administered to patients. For purposes of calculating conjugate number, a CDP-proteasome inhibitor conjugate molecule is a single CDP polymer with its covalently linked proteasome inhibitor.

Methods disclosed herein, provide for evaluating a particle, e.g., a nanoparticle, or preparation of particles, e.g., nanoparticles, wherein said particles, e.g., nanoparticles, comprise a CDP-proteasome inhibitor (such as a boronic acid proteasome inhibitor, e.g., bortezomib) conjugate. Generally, the method comprises providing a sample comprising a plurality of said particles, e.g., nanoparticles, determining a value for the number of CDP-proteasome inhibitor conjugates in a particle, e.g., nanoparticle, in the sample, to thereby evaluate a preparation of particles, e.g., nanoparticles.

Typically the value for a particle will be a function of the values obtained for a plurality of particles, e.g., the value will be the average of values determined for a plurality of particles.

In embodiments the method further comprises comparing the determined value with a reference value. The comparison can be used in a number of ways. By way of example, in response to a comparison or determination made in the method, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale. E.g., based on the result of the determination, or upon comparison to a reference standard, the batch from which the sample is taken can be processed, e.g., as just described.

In one embodiment, the CDP-proteasome inhibitor (such as a boronic acid containing proteasome inhibitor) conjugate (e.g., a CDP-bortezomib conjugate) forms or is provided as a particle (e.g., a nanoparticle) having a conjugate number described herein. By way of example, a CDP-proteasome inhibitor (such as a boronic acid containing proteasome inhibitor) conjugate (e.g., a CDP-bortezomib conjugate) forms, or is provided in, a nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g, a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g, a pharmaceutical preparation, wherein at least 60% of the particles in the preparation have a conjugate number of 1-5 or 2-5.

In an embodiment the conjugate number is from 1-100; 25 to 100; 50 to 100; 75-100; 25 to 75, 25 to 50, or 50 to 75; 25 to 40; 25 to 50; 30 to 50; 30 to 40; 30 to 75; 1 to 40; 1 to 30; 1 to 20; 1 to 15; 10 to 40; 10 to 30; 10 to 20; 10 to 15; 20 to 40; 20 to 30; or 20 to 25.

In an embodiment, the CDP-proteasome inhibitor (such as a boronic acid containing proteasome inhibitor) conjugate (e.g., a CDP-bortezomib conjugate) is administered as a nanoparticle or preparation of nanoparticles, e.g, a pharmaceutical preparation, wherein at least 60% of the particles in the preparation have a conjugate number of 1-100; 25 to 100; 50 to 100; 75-100; 25 to 75, 25 to 50, or 50 to 75; 25 to 40; 25 to 50; 30 to 50; 30 to 40; 30 to 75; 1 to 40; 1 to 30; 1 to 20; 1 to 15; 10 to 40; 10 to 30; 10 to 20; 10 to 15; 20 to 40; 20 to 30; or 20 to 25.

In another aspect, the invention features a method of evaluating a particle or a preparation of particles, wherein said particles comprise one or a plurality of CDP therapeutic agent conjugate molecules, e.g., CDP-proteasome inhibitor (such as a boronic acid containing proteasome inhibitor) conjugates (e.g., a CDP-bortezomib conjugate).

The method comprises:

providing a sample comprising one or a plurality of said particles;
determining a value for the number of CDP conjugate molecules in a particle in said sample (the conjugate number); and
thereby evaluating a preparation of particles.

In an embodiment the method comprises one or both of:

a) comparing said determined value with a reference value, e.g., a range of values, or
b) responsive to said determination, classifying said particles.

In an embodiment the particle is a nanoparticle.

In an embodiment the method further comprises comparing said determined value with a reference standard. In an embodiment the reference value can be selected from a value, e.g., a range provided herein, e.g., 1 or 2 to 8, 1 or 2 to 7, 1 or 2 to 6, 1 or 2 to 5, or 2-4.

In an embodiment the reference value can be selected from a value, e.g., a range, provided herein, e.g., 1-100; 25 to 100; 50 to 100; 75-100; 25 to 75, 25 to 50, or 50 to 75; 25 to 40; 25 to 50; 30 to 50; 30 to 40; 30 to 75; 1 to 40; 1 to 30; 1 to 20; 1 to 15; 10 to 40; 10 to 30; 10 to 20; 10 to 15; 20 to 40; 20 to 30; or 20 to 25.

In an embodiment, responsive to said comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment said CDP-proteasome inhibitor (such as a boronic acid containing proteasome inhibitor) conjugate (e.g., a CDP-bortezomib conjugate) is selected from those disclosed herein.

In an embodiment said proteasome inhibitor is selected from those disclosed herein.

In an embodiment said particle is selected from those disclosed herein.

In an embodiment, the determined value for conjugate number is compared with a reference, and responsive to said comparison said particle or preparation of particles is classified, e.g., as suitable for use in human subjects, not suitable for use in human subjects, suitable for sale, meeting a release specification, or not meeting a release specification.

In another aspect, the invention features a particle, e.g., a nanoparticle, comprising one or more CDP-proteasome inhibitor (such as a boronic acid containing proteasome inhibitor) conjugate (e.g., a CDP-bortezomib conjugate) described herein, having a conjugate number of: 2-50, 2-25, 2-10, or 2-5; 2-10, 10-20, 20-30, 40-50; 2-5, 2-4, or 3; or 1-2,2-3, 4-5, or 5-6. In an embodiment, said CDP-proteasome inhibitor conjugate is other than a CDP-boronoic acid conjugate.

As discussed above, conjugate number is defined as the number of CDP-proteasome inhibitor (such as a boronic acid containing proteasome inhibitor) conjugate (e.g., a CDP-bortezomib conjugate) molecules that self-assemble into a particle or nanoparticle, thus

C_{J}=[CDP-proteasome inhibitor conjugate]/P (or NP)

where C_{J} is conjugate number, [CDP-therapeutic agent conjugate]/is the number of CDP-proteasome inhibitor conjugate molecules, and P (or NP) is a single particle (or nanoparticle).

In order to arrive at a conjugate number, one determines the size of a particle, e.g., by dynamic light scattering. The size should be viscosity-adjusted size. The hydrodynamic volume of a CDP-proteasome inhibitor (such as a boronic acid containing proteasome inhibitor) conjugate (e.g., a CDP-bortezomib conjugate), or a molecule of similar molecular weight, is determined, to provide an expected hydrodynamic volume. Comparison of the expected hydrodynamic volume for the CDP-proteasome inhibitor conjugate with the volume for a particle of determined size provides conjugate number.

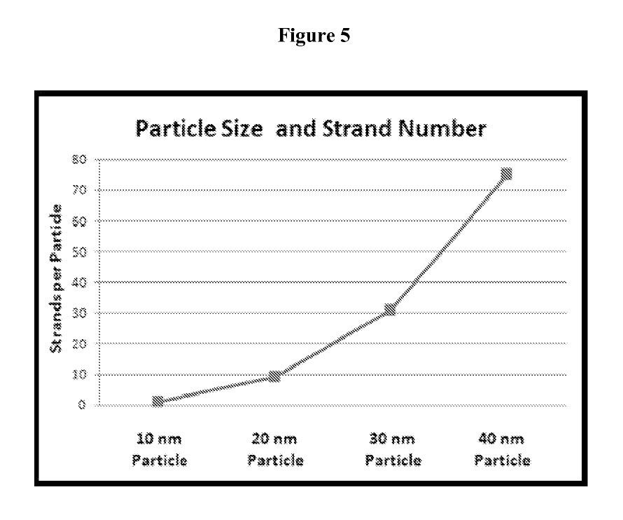

The determination of conjugate number can be illustrated with a conjugate having a molecular weight of 66.5 kDa. In this case, a number of fundamental assumptions are made in postulating nanoparticle characteristics. First, macromolecular volume estimates are based on work done with bovine serum albumin (BSA), a biological macromolecule of similar size to the 66.5 kDa conjugate (BSA MW=67 kDa). It has been demonstrated that a single strand of BSA has a hydrodynamic diameter of 9.5 nm. Simple volume calculations yield a volume of 3589 nm³. Extending this to the 66.5 kDa conjugate (with an average 30 nm particle) gives a volume of 33,485 nm³. With a particle size of 5-40 nm the conjugate number is 1-30. FIG. 5 shows a calculated strand dependence on particle size.

Polymer Polydispersity. Conjugate molecules fall within a range of molecular weights, with molecules of varying weight providing varying contributions to the particle diameter and conjugate number. Particles could form which are made up of strands which are larger and smaller than the average. Strands may also associate to a maximum size which could be shear-limited.

Particle Shape. Particle shape is assumed to be roughly spherical, and driven by either (or both) the hydrophobic region created by the CDP-proteasome inhibitor (such as a boronic acid containing proteasome inhibitor) conjugate (e.g., a CDP-bortezomib conjugate), or by guest-host complexation with pendant therapeutic agent molecules making inclusion complexes with CDs from adjacent strands. One critical point of note is that as a drug product, the nanoparticles are in a somewhat controlled environment as they are characterized. Upon administration, myriad possibilities exist for interaction with endogenous substances: inclusion complexes of circulating small molecules, metal ion complexation with the PEG subunits, etc. Any one of these, or all of them in concert, could dramatically alter the nanoparticle structure and function.

### CDPs, Methods of Making Same, and Methods of Conjugating CDPs to Proteasome Inhibitor

Generally, the CDP-proteasome inhibitor (such as a boronic acid containing proteasome inhibitor) conjugate, e.g., a CDP-bortezomib conjugate, described herein can be prepared in one of two ways: monomers bearing proteasome inhibitor (such as a boronic acid containing proteasome inhibitor, e.g., bortezomib), targeting ligands, and/or cyclodextrin moieties can be polymerized, or polymer backbones can be derivatized with proteasome inhibitor (such as a boronic acid containing proteasome inhibitor, e.g., bortezomib), targeting ligands, and/or cyclodextrin moieties.

Thus, in one embodiment, the synthesis of CDP-proteasome inhibitor (such as a boronic acid containing proteasome inhibitor) conjugates, e.g., a CDP-bortezomib conjugate, can be accomplished by reacting monomers M-L-CD and M-L-D (and, optionally, M-L-T), wherein

CD represents a cyclic moiety, such as a cyclodextrin molecule, or derivative thereof;

L, independently for each occurrence, may be absent or represents a linker group;

D, independently for each occurrence, represents the same or different proteasome inhibitor (such as a boronic acid containing proteasome inhibitor, e.g., bortezomib) or prodrug thereof;

T, independently for each occurrence, represents the same or different targeting ligand or precursor thereof; and

M represents a monomer subunit bearing one or more reactive moieties capable of undergoing a polymerization reaction with one or more other M in the monomers in the reaction mixture, under conditions that cause polymerization of the monomers to take place.

In some embodiments, one or more of the proteasome inhibitor (such as a boronic acid containing proteasome inhibitor, e.g., bortezomib) moieties in the CDP-proteasome inhibitor (such as a boronic acid containing proteasome inhibitor) conjugate, e.g., a CDP-bortezomib conjugate, conjugate can be replaced with another therapeutic agent, e.g., another anticancer agent or anti-inflammatory agent.

In certain embodiments, the reaction mixture may further comprise monomers that do not bear CD, T, or D moieties, e.g., to space the derivatized monomer units throughout the polymer.

In an alternative embodiment, the invention contemplates synthesizing a CDP-proteasome inhibitor (such as a boronic acid containing proteasome inhibitor) conjugate, e.g., a CDP-bortezomib conjugate, by reacting a polymer P (the polymer bearing a plurality of reactive groups, such as carboxylic acids, alcohols, thiols, amines, epoxides, etc.) with grafting agents X-L-CD and/or Y-L-D (and, optionally, Z-L-T), wherein

CD represents a cyclic moiety, such as a cyclodextrin molecule, or derivative thereof;

L, independently for each occurrence, may be absent or represents a linker group;

D, independently for each occurrence, represents the same or different proteasome inhibitor (such as a boronic acid containing proteasome inhibitor, e.g., bortezomib) or prodrug thereof;

T, independently for each occurrence, represents the same or different targeting ligand or precursor thereof;

X, independently for each occurrence, represents a reactive group, such as carboxylic acids, alcohols, thiols, amines, epoxides, etc., capable of forming a covalent bond with a reactive group of the polymer; and

Y and Z, independently for each occurrence, represent inclusion hosts or reactive groups, such as carboxylic acids, alcohols, thiols, amines, epoxides, etc., capable of forming a covalent bond with a reactive group of the polymer or inclusion complexes with CD moieties grafted to the polymer, under conditions that cause the grafting agents to form covalent bonds and/or inclusion complexes, as appropriate, with the polymer or moieties grafted to the polymer.

In some embodiments, one or more of the proteasome inhibitor (such as a boronic acid containing proteasome inhibitor, e.g., bortezomib) moieties in CDP-proteasome inhibitor (such as a boronic acid containing proteasome inhibitor) conjugate, e.g., a CDP-bortezomib conjugate, can be replaced with another therapeutic agent, e.g., another anticancer agent or anti-inflammatory agent.

For example, if the CDP includes alcohols, thiols, or amines as reactive groups, the grafting agents may include reactive groups that react with them, such as isocyanates, isothiocyanates, acid chlorides, acid anhydrides, epoxides, ketenes, sulfonyl chlorides, activated carboxylic acids (e.g., carboxylic acids treated with an activating agent such as PyBrOP, carbonyldiimidazole, or another reagent that reacts with a carboxylic acid to form a moiety susceptible to nucleophilic attack), or other electrophilic moieties known to those of skill in the art. In certain embodiments, a catalyst may be needed to cause the reaction to take place (e.g., a Lewis acid, a transition metal catalyst, an amine base, etc.) as will be understood by those of skill in the art.

In certain embodiments, the different grafting agents are reacted with the polymer simultaneously or substantially simultaneously (e.g., in a one-pot reaction), or are reacted sequentially with the polymer (optionally with a purification and/or wash step between reactions).

Another aspect of the present invention is a method for manufacturing the linear or branched CDPs and CDP-proteasome inhibitor (such as a boronic acid containing proteasome inhibitor) conjugate, e.g., a CDP-bortezomib conjugate, as described herein. While the discussion below focuses on the preparation of linear cyclodextrin molecules, one skilled in the art would readily recognize that the methods described can be adapted for producing branched polymers by choosing an appropriate comonomer precursor.

Accordingly, one embodiment of the invention is a method of preparing a linear CDP. According to the invention, a linear CDP may be prepared by copolymerizing a cyclodextrin monomer precursor disubstituted with one or more appropriate leaving groups with a comonomer precursor capable of displacing the leaving groups. The leaving group, which may be the same or different, may be any leaving group known in the art which may be displaced upon copolymerization with a comonomer precursor. In a preferred embodiment, a linear CDP may be prepared by iodinating a cyclodextrin monomer precursor to form a diiodinated cyclodextrin monomer precursor and copolymerizing the diiodinated cyclodextrin monomer precursor with a comonomer precursor to form a linear CDP having a repeating unit of formula I or II, provided in the section entitles “CDP-proteasome inhibitor conjugates” or a combination thereof, each as described above. In some embodiments, the cyclodextrin moiety precursors are in a composition, the composition being substantially free of cyclodextrin moieties having other than two positions modified to bear a reactive site (e.g., 1, 3, 4, 5, 6, or 7). While examples presented below discuss iodinated cyclodextrin moieties, one skilled in the art would readily recognize that the present invention contemplates and encompasses cyclodextrin moieties wherein other leaving groups such as alkyl and aryl sulfonate may be present instead of iodo groups. In a preferred embodiment, a method of preparing a linear cyclodextrin copolymer of the invention by iodinating a cyclodextrin monomer precursor as described above to form a diiodinated cyclodextrin monomer precursor of formula IVa, IVb, IVc or a mixture thereof:

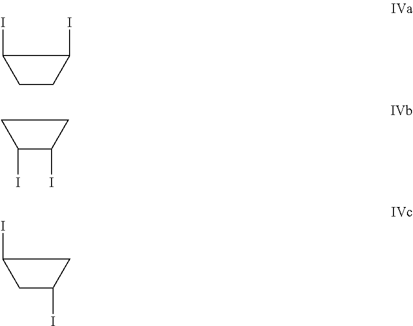

In some embodiments, the iodine moieties as shown on the cyclodextrin moieties are positioned such that the derivatization on the cyclodextrin is on the A and D glucopyranose moieties. In some embodiments, the iodine moieties as shown on the cyclodextrin moieties are positioned in such that the derivatization on the cyclodextrin is on the A and C glucopyranose moieties. In some embodiments, the iodine moieties as shown on the cyclodextrin moieties are positioned in such that the derivatization on the cyclodextrin is on the A and F glucopyranose moieties. In some embodiments, the iodine moieties as shown on the cyclodextrin moieties are positioned in such that the derivatization on the cyclodextrin is on the A and E glucopyranose moieties.

The diiodinated cyclodextrin may be prepared by any means known in the art. (Tabushi et al. J. Am. Chem. 106, 5267-5270 (1984); Tabushi et al. J. Am. Chem. 106, 4580-4584 (1984)). For example, β-cyclodextrin may be reacted with biphenyl-4,4′-disulfonyl chloride in the presence of anhydrous pyridine to form a biphenyl-4,4′-disulfonyl chloride capped β-cyclodextrin which may then be reacted with potassium iodide to produce diiodo-β-cyclodextrin. The cyclodextrin monomer precursor is iodinated at only two positions. By copolymerizing the diiodinated cyclodextrin monomer precursor with a comonomer precursor, as described above, a linear cyclodextrin polymer having a repeating unit of Formula Ia, Ib, or a combination thereof, also as described above, may be prepared. If appropriate, the iodine or iodo groups may be replaced with other known leaving groups.

Also according to the invention, the iodo groups or other appropriate leaving group may be displaced with a group that permits reaction with a comonomer precursor, as described above. For example, a diiodinated cyclodextrin monomer precursor of formula IVa, IVb, IVc or a mixture thereof may be aminated to form a diaminated cyclodextrin monomer precursor of formula Va, Vb, Vc or a mixture thereof:

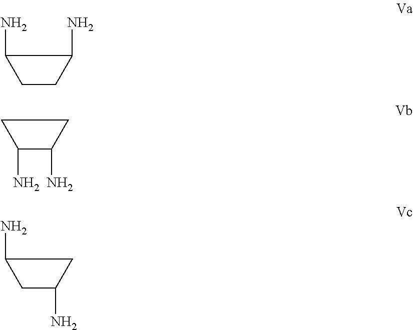

In some embodiments, the amino moieties as shown on the cyclodextrin moieties are positioned such that the derivatization on the cyclodextrin is on the A and D glucopyranose moieties. In some embodiments, the amino moieties as shown on the cyclodextrin moieties are positioned in such that the derivatization on the cyclodextrin is on the A and C glucopyranose moieties. In some embodiments, the amino moieties as shown on the cyclodextrin moieties are positioned in such that the derivatization on the cyclodextrin is on the A and F glucopyranose moieties. In some embodiments, the amino moieties as shown on the cyclodextrin moieties are positioned in such that the derivatization on the cyclodextrin is on the A and E glucopyranose moieties.

The diaminated cyclodextrin monomer precursor may be prepared by any means known in the art. (Tabushi et al. Tetrahedron Lett. 18:11527-1530 (1977); Mungall et al., J. Org. Chem. 16591662 (1975)). For example, a diiodo-β-cyclodextrin may be reacted with sodium azide and then reduced to form a diamino-β-cyclodextrin). The cyclodextrin monomer precursor is aminated at only two positions. The diaminated cyclodextrin monomer precursor may then be copolymerized with a comonomer precursor, as described above, to produce a linear cyclodextrin copolymer having a repeating unit of formula I-II provided in the section entitles “CDP-proteasome inhibitor conjugates” or a combination thereof, also as described above. However, the amino functionality of a diaminated cyclodextrin monomer precursor need not be directly attached to the cyclodextrin moiety. Alternatively, the amino functionality or another nucleophilic functionality may be introduced by displacement of the iodo or other appropriate leaving groups of a cyclodextrin monomer precursor with amino group containing moieties such as, for example, HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine to form a diaminated cyclodextrin monomer precursor of formula Vd, Ve, Vf or a mixture thereof:

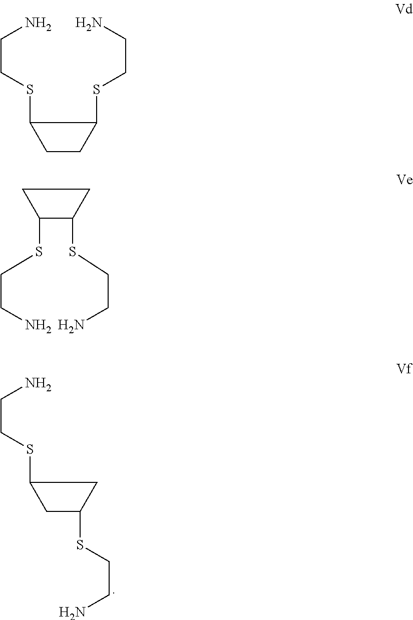

In some embodiments, the —SCH₂CH₂NH₂ moieties as shown on the cyclodextrin moieties are positioned such that the derivatization on the cyclodextrin is on the A and D glucopyranose moieties. In some embodiments, the —SCH₂CH₂NH₂ moieties as shown on the cyclodextrin moieties are positioned in such that the derivatization on the cyclodextrin is on the A and C glucopyranose moieties. In some embodiments, the —SCH₂CH₂NH₂ moieties as shown on the cyclodextrin moieties are positioned in such that the derivatization on the cyclodextrin is on the A and F glucopyranose moieties. In some embodiments, the —SCH₂CH₂NH₂ moieties as shown on the cyclodextrin moieties are positioned in such that the derivatization on the cyclodextrin is on the A and E glucopyranose moieties.

A linear oxidized CDP may also be prepared by oxidizing a reduced linear cyclodextrin-containing copolymer as described below. This method may be performed as long as the comonomer does not contain an oxidation sensitive moiety or group such as, for example, a thiol.

A linear CDP of the invention may be oxidized so as to introduce at least one oxidized cyclodextrin monomer into the copolymer such that the oxidized cyclodextrin monomer is an integral part of the polymer backbone. A linear CDP which contains at least one oxidized cyclodextrin monomer is defined as a linear oxidized cyclodextrin copolymer or a linear oxidized cyclodextrin-containing polymer. The cyclodextrin monomer may be oxidized on either the secondary or primary hydroxyl side of the cyclodextrin moiety. If more than one oxidized cyclodextrin monomer is present in a linear oxidized cyclodextrin copolymer of the invention, the same or different cyclodextrin monomers oxidized on either the primary hydroxyl side, the secondary hydroxyl side, or both may be present. For illustration purposes, a linear oxidized cyclodextrin copolymer with oxidized secondary hydroxyl groups has, for example, at least one unit of formula VIa or VIb:

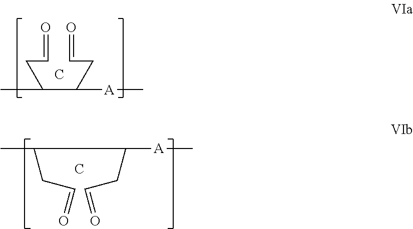

In formulae VIa and VIb, C is a substituted or unsubstituted oxidized cyclodextrin monomer and the comonomer (i.e., shown herein as A) is a comonomer bound, i.e., covalently bound, to the oxidized cyclodextrin C. Also in formulae Via and VIb, oxidation of the secondary hydroxyl groups leads to ring opening of the cyclodextrin moiety and the formation of aldehyde groups.

A linear oxidized CDP copolymer may be prepared by oxidation of a linear cyclodextrin copolymer as discussed above. Oxidation of a linear cyclodextrin copolymer of the invention may be accomplished by oxidation techniques known in the art. (Hisamatsu et al., Starch 44:188-191 (1992)). Preferably, an oxidant such as, for example, sodium periodate is used. It would be understood by one of ordinary skill in the art that under standard oxidation conditions that the degree of oxidation may vary or be varied per copolymer. Thus in one embodiment of the invention, a CDP may contain one oxidized cyclodextrin monomer. In another embodiment, substantially all cyclodextrin monomers of the copolymer would be oxidized.

Another method of preparing a linear oxidized CDP involves the oxidation of a diiodinated or diaminated cyclodextrin monomer precursor, as described above, to form an oxidized diiodinated or diaminated cyclodextrin monomer precursor and copolymerization of the oxidized diiodinated or diaminated cyclodextrin monomer precursor with a comonomer precursor. In a preferred embodiment, an oxidized diiodinated cyclodextrin monomer precursor of formula VIIa, VIIb, VIIc, or a mixture thereof:

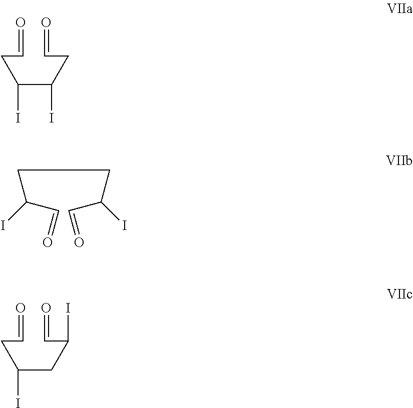

may be prepared by oxidation of a diiodinated cyclodextrin monomer precursor of formulae IVa, IVb, IVc, or a mixture thereof, as described above.

In another preferred embodiment, an oxidized diaminated cyclodextrin monomer precursor of formula VIIIa, VIIIb, VIIIc or a mixture thereof:

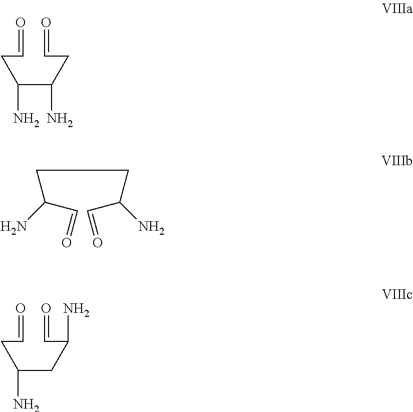

may be prepared by amination of an oxidized diiodinated cyclodextrin monomer precursor of formulae VIIa, VIIb, VIIc, or a mixture thereof, as described above.

In still another preferred embodiment, an oxidized diaminated cyclodextrin monomer precursor of formula IXa, IXb, IXc or a mixture thereof:

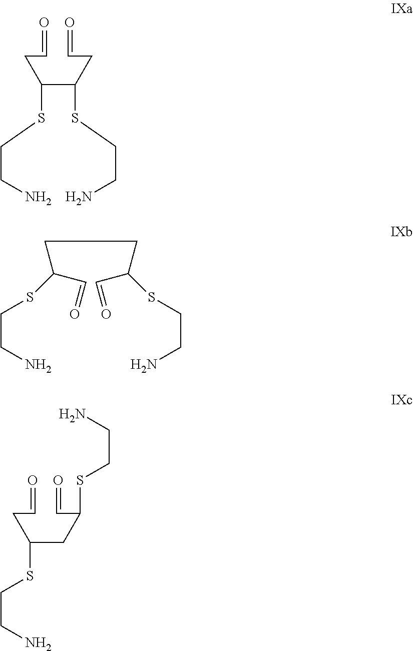

may be prepared by displacement of the iodo or other appropriate leaving groups of an oxidized cyclodextrin monomer precursor disubstituted with an iodo or other appropriate leaving group with the amino or other nucleophilic group containing moiety such as, e.g. HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine.

Alternatively, an oxidized diiodinated or diaminated cyclodextrin monomer precursor, as described above, may be prepared by oxidizing a cyclodextrin monomer precursor to form an oxidized cyclodextrin monomer precursor and then diiodinating and/or diaminating the oxidized cyclodextrin monomer, as described above. As discussed above, the cyclodextrin moiety may be modified with other leaving groups other than iodo groups and other amino group containing functionalities. The oxidized diiodinated or diaminated cyclodextrin monomer precursor may then be copolymerized with a comonomer precursor, as described above, to form a linear oxidized cyclodextrin copolymer of the invention.

A linear oxidized CDP may also be further modified by attachment of at least one ligand to the copolymer. The ligand is as described above.

In some embodiments, a CDP comprises: cyclodextrin moieties, and comonomers which do not contain cyclodextrin moieties (comonomers), and wherein the CDP comprises at least four, five six, seven, eight, nine, ten, eleven, twelve, thirteen, fourteen, fifteen, sixteen, seventeen, eighteen, nineteen or twenty cyclodextrin moieties and at least four, five six, seven, eight, nine, ten, eleven, twelve, thirteen, fourteen, fifteen, sixteen, seventeen, eighteen, nineteen or twenty comonomers.

In some embodiments, the at least four, five six, seven, eight, etc., cyclodextrin moieties and at least four, five six, seven, eight, nine, ten, eleven, twelve, thirteen, fourteen, fifteen, sixteen, seventeen, eighteen, nineteen or twenty comonomers alternate in the water soluble linear polymer.

In some embodiments, the cyclodextrin moieties comprise linkers to which therapeutic agents may be further linked.

In some embodiments, the CDP has no proteasome inhibitor attached. In some embodiments, the CDP has a plurality (i.e., more than one) of proteasome inhibitors attached (e.g., through a linker). In some embodiments, the proteasome inhibitors are attached via a second linker

In some embodiments, the comonomer is a compound containing residues of least two functional groups through which reaction and thus linkage of the cyclodextrin monomers is achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the residues of the two functional groups are the same and are located at termini of the comonomer. In some embodiments, a comonomer contains one or more pendant groups with at least one functional group through which reaction and thus linkage of a proteasome inhibitor (such as a boronic acid containing proteasome inhibitor, e.g., bortezomib) can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient proteasome inhibitor (such as a boronic acid containing proteasome inhibitor, e.g., bortezomib) such that up to at least 5%, 10%, 15%, 20%, 25%, 30%, or even 35% by weight of the water soluble linear polymer, when conjugated, is proteasome inhibitor.

In some embodiments, the molecular weight of the CDP is 10,000-500,000 Da, e.g., about 30,000 to about 100,000 Da.

In some embodiments, the cyclodextrin moieties make up at least about 2%, 5%, 10%, 11%, 12%, 13%, 14%, 15%, 16%, 17%, 18%, 19%, 20%, 30%, 50% or 80% of the polymer by weight.

In some embodiments, the CDP is made by a method comprising providing cyclodextrin moiety precursors modified to bear one reactive site at each of exactly two positions, and reacting the cyclodextrin moiety with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and comonomer is produced.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain.

In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁CO—, —C(O)NR₁—, —S(O)ₙ— (wherein n is 0, 1, or 2), —OC(O)—NR₁, —NR₁₁—C(NR₁)—NR₁—, and —B(OR₁)—; and R₁, independently for each occurrence, represents H or a lower alkyl.

In some embodiments, the CDP is a polymer of the following formula:

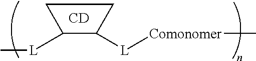

wherein each L is independently a linker, each comonomer is independently a comonomer described herein, and n is at least 4, 5, 6, 7, 8, 9, 10, 11, 12, 13, 14, 15, 16, 17, 18, 19 or 20. In some embodiments, the molecular weight of the comonomer is from about 2000 to about 5000 Da (e.g., from about 3000 to about 4000 Da (e.g., about 3400 Da).

In some embodiments, the CDP is a polymer of the following formula:

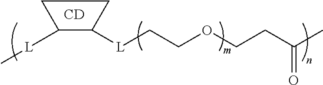

wherein each L is independently a linker,

wherein the group

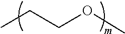

has a Mw of 3.4 kDa or less and n is at least 4, 5, 6, 7, 8, 9, 10, 11, 12, 13, 14, 15, 16, 17, 18, 19 or 20.

In some embodiments,

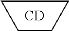

is alpha, beta or gamma cyclodextrin, e.g., beta cyclodextrin.

In some embodiments, each L independently comprises an amino acid or a derivative thereof. In some embodiments, at least one L comprises cysteine or a derivative thereof. In some embodiments, each L comprises cysteine. In some embodiments, each L is cysteine and the cysteine is connected to the CD by way of a thiol linkage.

In some embodiments, the CDP is a polymer of the following formula:

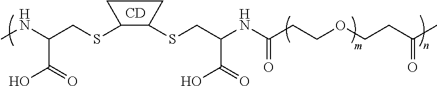

wherein the group

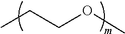

has a Mw of 3.4 kDa or less and n is at least 4, 5, 6, 7, 8, 9, 10, 11, 12, 13, 14, 15, 16, 17, 18, 19 or 20.

In some embodiments,

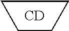

is alpha, beta or gamma cyclodextrin, e.g., beta cyclodextrin.

In some embodiments, the CDP is a polymer of the following formula:

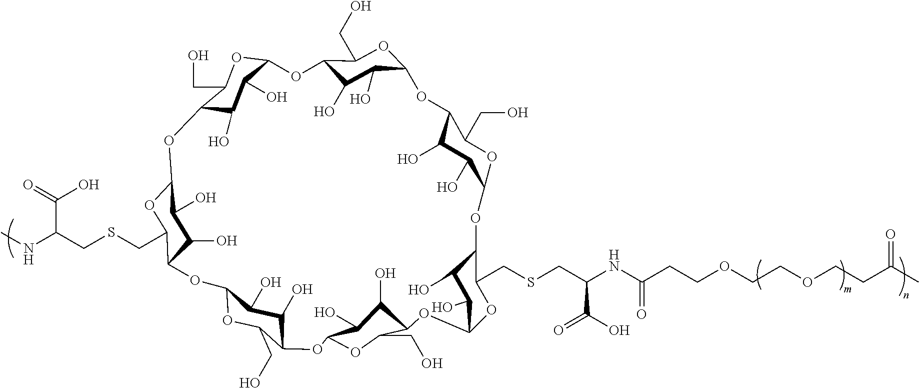

wherein the group

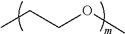

has a Mw of 3.4 kDa or less and n is at least 4, 5, 6, 7, 8, 9, 10, 11, 12, 13, 14, 15, 16, 17, 18, 19 or 20.

In some embodiments, the group

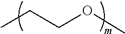

has a Mw of 3.4 kDa and the Mw of the compound as a whole is from 27 kDa to 99.6 kDa.

The CDPs described herein can be made using a variety of methods including those described herein. In some embodiments, a CDP can be made by: providing cyclodextrin moiety precursors; providing comonomer precursors which do not contain cyclodextrin moieties (comonomer precursors); and copolymerizing the said cyclodextrin moiety precursors and comonomer precursors to thereby make a CDP wherein CDP comprises at least four, five six, seven, eight, etc., cyclodextrin moieties and at least four, five six, seven, eight, etc., comonomers.

In some embodiments, the at least four, five, six, seven or eight cyclodextrin moieties and at least four, five, six, seven or eight comonomers alternate in the water soluble linear polymer. In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the cyclodextrin comonomers comprise linkers to which proteasome inhibitors may be further linked. In some embodiments, the proteasome inhibitor are linked via second linkers.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C1-C10 alkyl, or arylalkyl optionally containing one or more heteroatoms within the chain or ring. In some embodiments, the cyclodextrin moiety comprises an alpha, beta, or gamma cyclodextrin moiety.

In some embodiments, the CDP is suitable for the attachment of sufficient proteasome inhibitor such that up to at least 3%, 5%, 10%, 15%, 20%, 25%, 30%, or even 35% by weight of the CDP, when conjugated, is proteasome inhibitor.

In some embodiments, the CDP has a molecular weight of 10,000-500,000. In some embodiments, the cyclodextrin moieties make up at least about 2%, 5%, 10%, 20%, 30%, 50% or 80% of the CDP by weight.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain. the CDP comprises a comonomer selected from the group consisting of a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In some embodiments, a CDP of the following formula can be made by the scheme below:

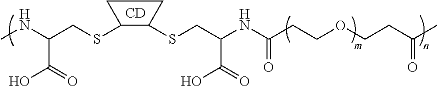

providing a polymer of formula A and formula B:

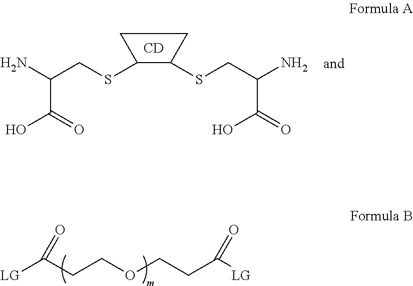

wherein LG is a leaving group;

and contacting the polymers under conditions that allow for the formation of a covalent bond between the polymers of formula A and B, to form a polymer of the following formula:

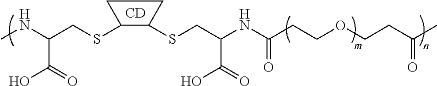

wherein the group

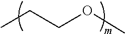

has a Mw of 3.4 kDa or less and n is at least four.

In some embodiments, Formula B is

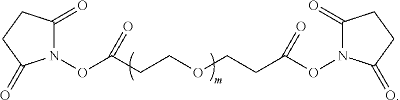

In some embodiments, the group

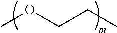

has a Mw of 3.4 kDa and the Mw of the compound is from 27 kDa to 99.6 kDa.

In some embodiments, the polymers of formula A and formula B are contacted in the presence of a base. In some embodiments, the base is an amine containing base. In some embodiments, the base is DEA.

In some embodiments, a CDP of the following formula can be made by the scheme below:

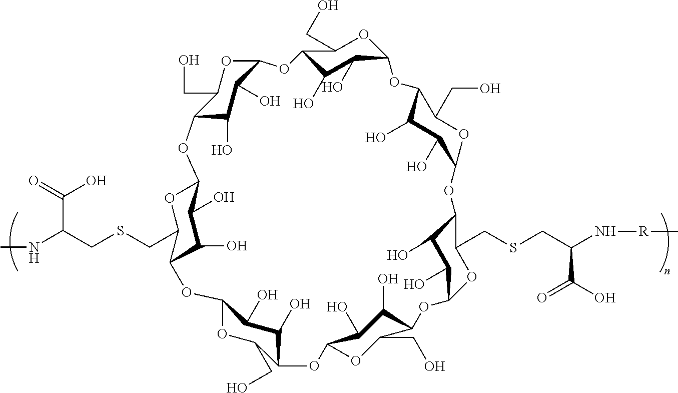

wherein R is of the form:

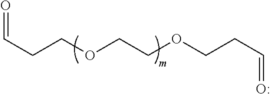

comprising the steps of:

reacting a compound of the formula below:

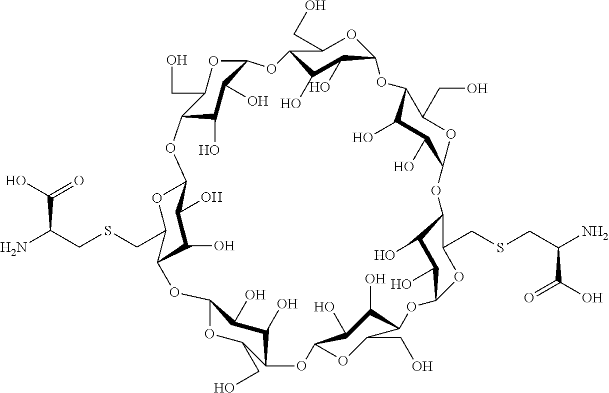

with a compound of the formula below:

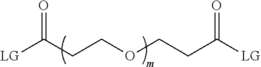

wherein the group

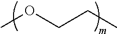

has a Mw of 3.4 kDa or less and n is at least four,

in the presence of a non-nucleophilic organic base in a solvent.

In some embodiments,

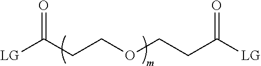

is

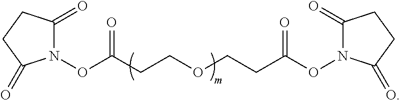

In some embodiments, the solvent is a polar aprotic solvent. In some embodiments, the solvent is DMSO.

In some embodiments, the method also includes the steps of dialysis; and lyophylization.

In some embodiments, a CDP provided below can be made by the following scheme:

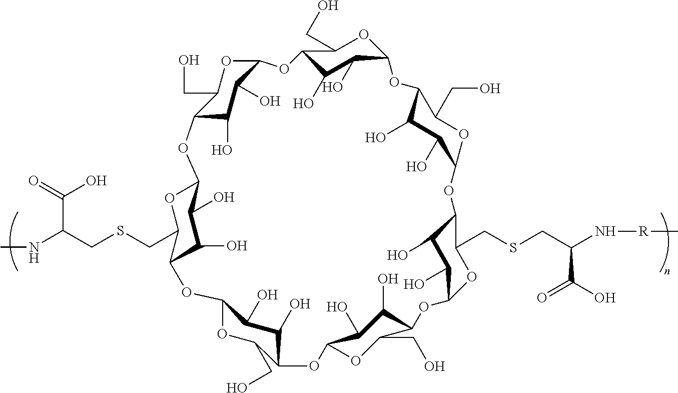

wherein R is of the form:

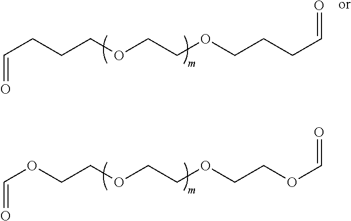

comprising the steps of:

reacting a compound of the formula below:

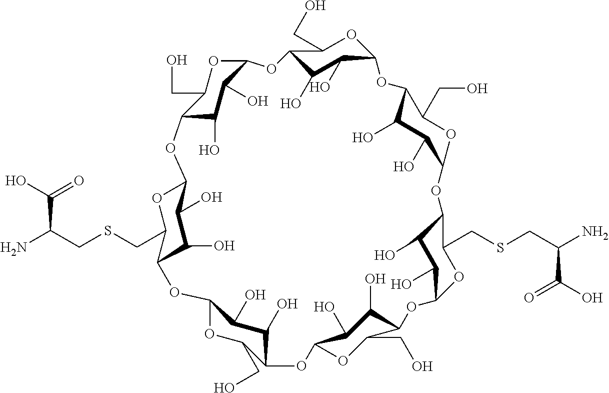

with a compound of the formula below:

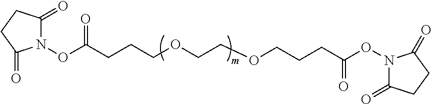

wherein the group

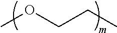

has a Mw of 3.4 kDa or less and n is at least four,

or with a compound provided below:

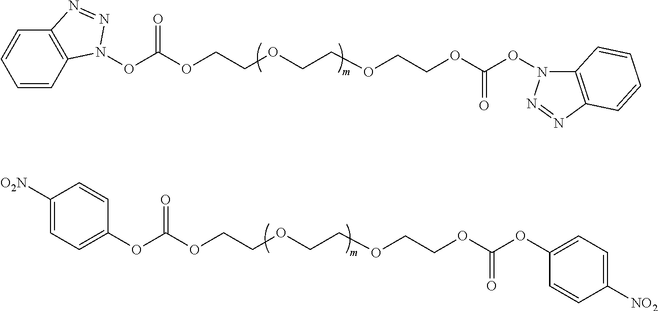

wherein the group

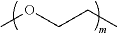

has a Mw of 3.4 kDa;

in the presence of a non-nucleophilic organic base in DMSO;

and dialyzing and lyophilizing the following polymer

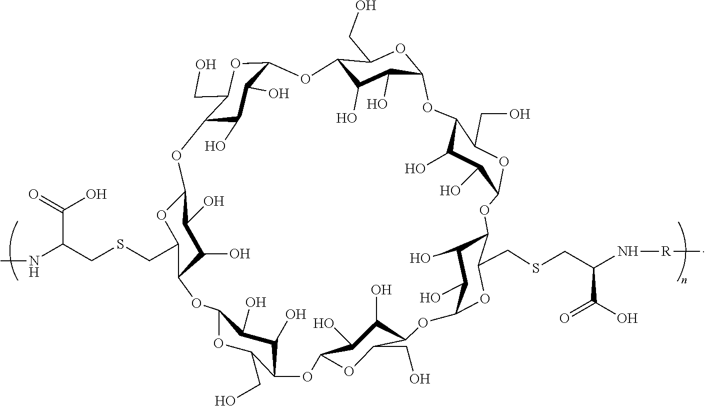

A CDP described herein may be attached to or grafted onto a substrate. The substrate may be any substrate as recognized by those of ordinary skill in the art. In another preferred embodiment of the invention, a CDP may be crosslinked to a polymer to form, respectively, a crosslinked cyclodextrin copolymer or a crosslinked oxidized cyclodextrin copolymer. The polymer may be any polymer capable of crosslinking with a CDP (e.g., polyethylene glycol (PEG) polymer, polyethylene polymer). The polymer may also be the same or different CDP. Thus; for example, a linear CDP may be crosslinked to any polymer including, but not limited to, itself, another linear CDP, and a linear oxidized CDP. A crosslinked linear CDP may be prepared by reacting a linear CDP with a polymer in the presence of a crosslinking agent. A crosslinked linear oxidized CDP may be prepared by reacting a linear oxidized CDP with a polymer in the presence of an appropriate crosslinking agent. The crosslinking agent may be any crosslinking agent known in the art. Examples of crosslinking agents include dihydrazides and disulfides. In a preferred embodiment, the crosslinking agent is a labile group such that a crosslinked copolymer may be uncrosslinked if desired.

A linear CDP and a linear oxidized CDP may be characterized by any means known in the art. Such characterization methods or techniques include, but are not limited to, gel permeation chromatography (GPC), matrix assisted laser desorption ionization-time of flight mass spectrometry (MALDI-TOF Mass spec), ¹H and ¹³C NMR, light scattering and titration.

The invention also provides a cyclodextrin composition containing at least one linear CDP and at least one linear oxidized CDP as described above. Accordingly, either or both of the linear CDP and linear oxidized CDP may be crosslinked to another polymer and/or bound to a ligand as described above. Therapeutic compositions according to the invention contain a proteasome inhibitor and a linear CDP or a linear oxidized CDP, including crosslinked copolymers. A linear CDP, a linear oxidized CDP and their crosslinked derivatives are as described above. The proteasome inhibitor may be any synthetic, semi-synthetic or naturally occurring biologically active proteasome inhibitor, including those known in the art.

One aspect of the present invention contemplates attaching a proteasome inhibitor to a CDP for delivery of a proteasome inhibitor. The present invention discloses various types of linear, branched, or grafted CDPs wherein a proteasome inhibitor is covalently bound to the polymer. In certain embodiments, the proteasome inhibitor is covalently linked via a biohydrolyzable bond, for example, an ester, amide, carbamates, or carbonate.

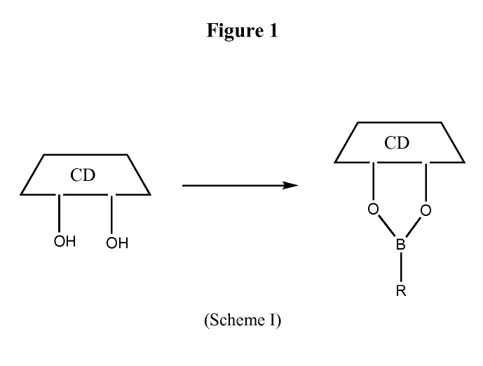

An exemplary synthetic scheme for covalently bonding a derivatized CD to a boronic acid is shown in FIG. 1 (Scheme I), wherein the boronic acid is complexed with any of the 1,2-diols situated on the rim of the CD (with R representing the remainder of the boronic acid).

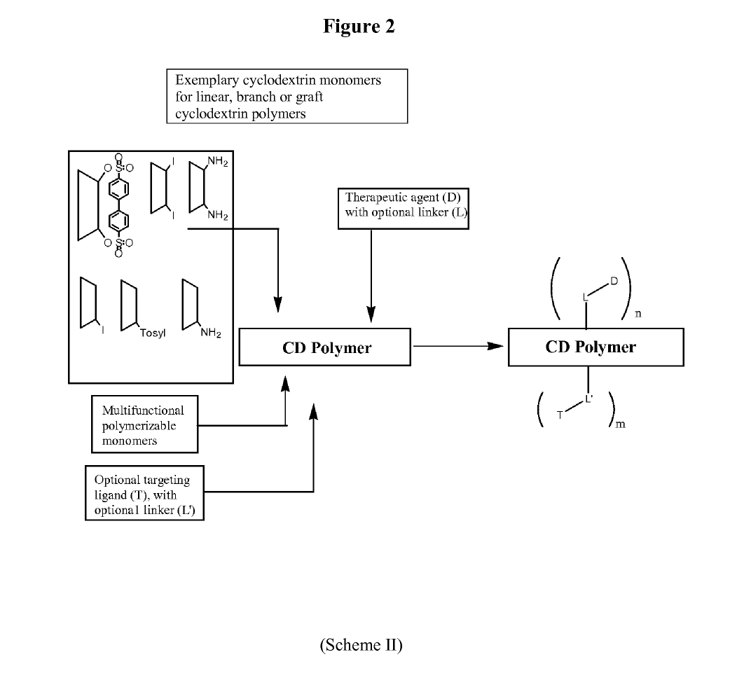

A general strategy for synthesizing linear, branched or grafted cyclodextrin-containing polymers (CDPs) for loading a boronic acid, and an optional targeting ligand is shown in FIG. 2 (Scheme II).

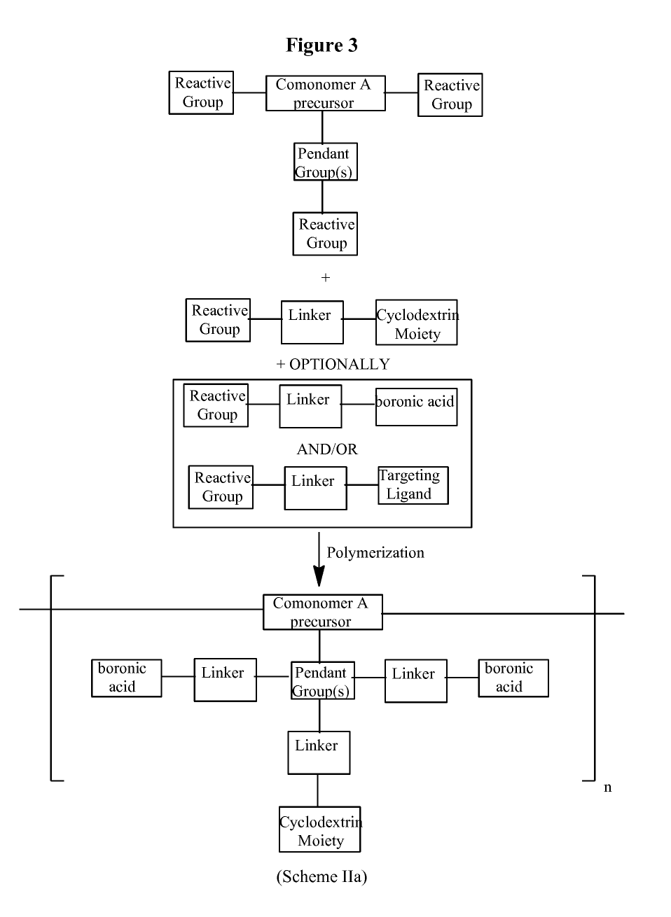

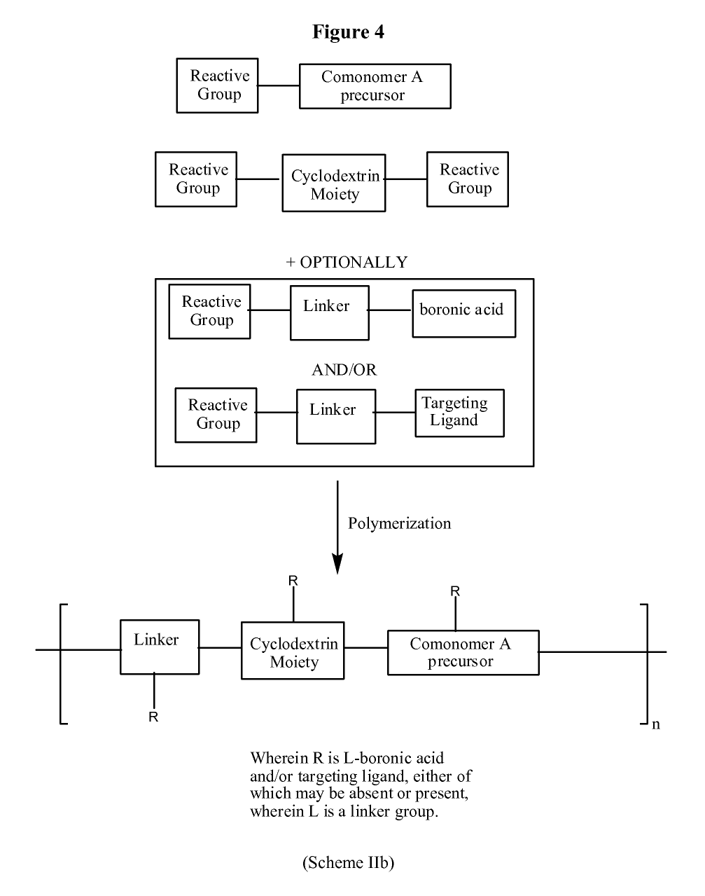

To illustrate further, comonomer precursors (shown in the FIG. 3 as A), cyclodextrin moieties, boronic acids, and/or targeting ligands may be assembled as shown in FIGS. 3 and 4 (Schemes IIa-IIb). Note that in schemes IIa-IIb, in any given reaction there may be more than one comonomer precursor, cyclodextrin moiety, therapeutic agent or targeting ligand that is of the same type or different. Furthermore, prior to polymerization, one or more comonomer precursor, cyclodextrin moiety, therapeutic agent or targeting ligand may be covalently linked with each other in one or more separate step. The scheme as provided above includes embodiments, where not all available positions for attachment of the boronic acid are occupied on the CDP. For example, in some embodiments, less than all of the available points of attachments are reacted, leaving less than 100% yield of the boronic acid onto the polymer. Accordingly, the loading of the boronic acid onto the polymer can vary. This is also the case regarding a targeting agent when a targeting agent is included.

A general scheme for graft polymers is shown in FIG. 3 (Scheme IIa). The comonomer A precursor, cyclodextrin moiety, boronic acid and optional targeting ligand are as defined above. Furthermore, one skilled in the art may choose from a variety of reactive groups, e.g., hydroxyls, carboxyls, halides, amines, and activated ethenes, ethynes, or aromatic groups in order achieve polymerization. For further examples of reactive groups are disclosed in Advanced Organic Chemistry: Reactions, Mechanisms, and Structure, 5th Edition, 2000.

In some embodiments, one or more of the boronic acid moieties and the associated linker in the CDP-boronic acid conjugate can be replaced with another therapeutic agent, e.g., another anticancer agent or anti-inflammatory agent, along with an optional linker.

A general scheme of preparing linear CDPs is shown in FIG. 3 (Scheme IIb). One skilled in the art would recognize that by choosing a comonomer A precursor that has multiple reactive groups polymer branching can be achieved.

In some embodiments, one or more of the boronic acid moieties in the CDP-boronic acid conjugate can be replaced with another therapeutic agent, e.g., another anticancer agent or anti-inflammatory agent, along with an optional linker.

Examples of different ways of synthesizing CDP-boronic acid conjugates are shown in Schemes III-VIII below. In each of Schemes III-VIII, one or more of the boronic acid moieties in the CDP-boronic acid conjugate can be replaced with another therapeutic agent, e.g., another anticancer agent or anti-inflammatory agent, along with an optional linker.

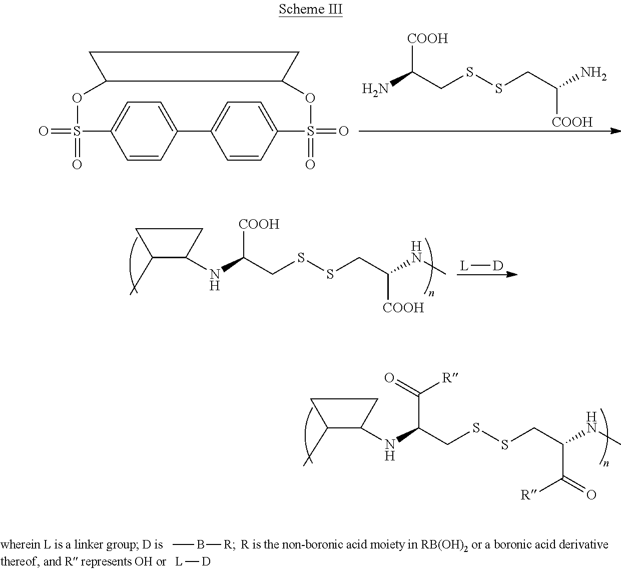

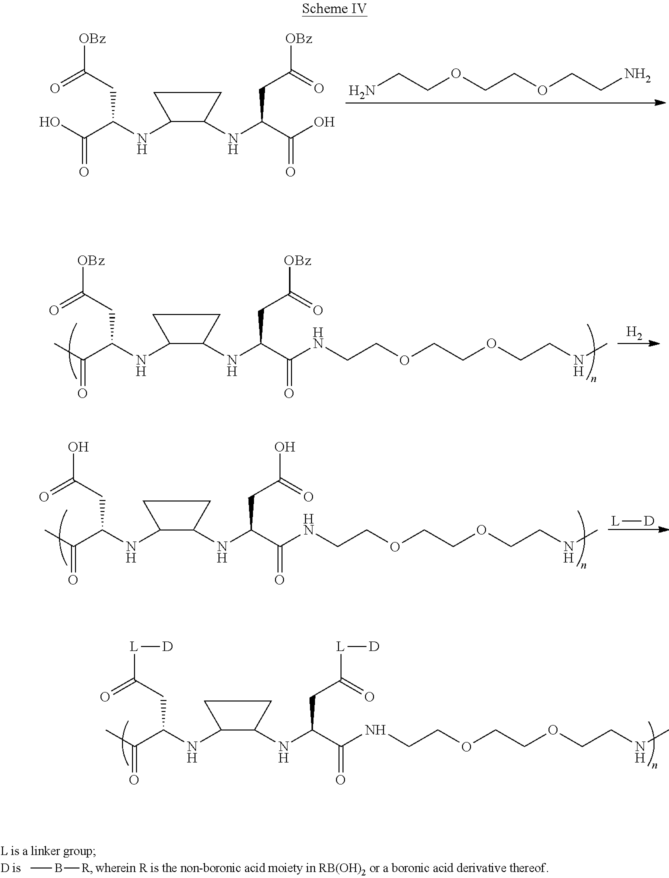

Scheme IV, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

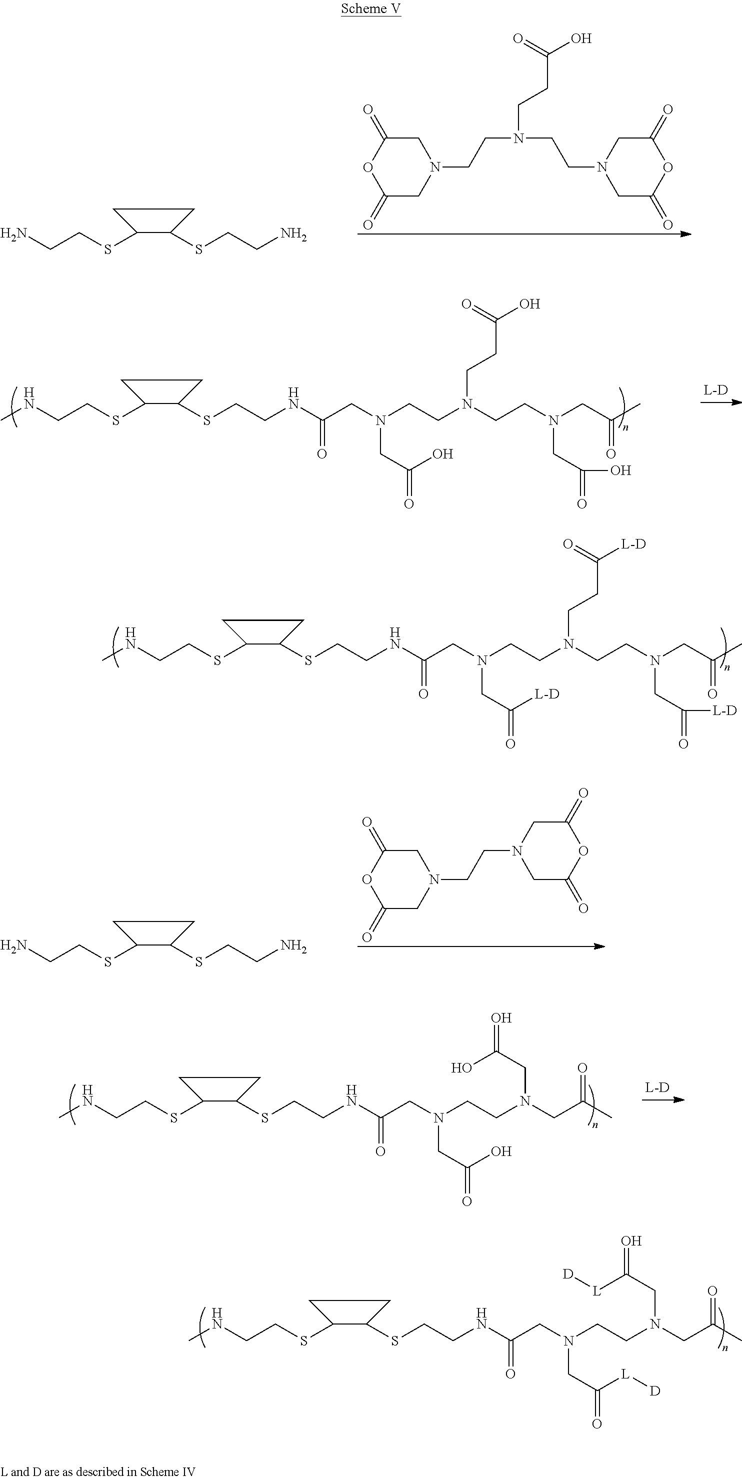

Scheme V, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

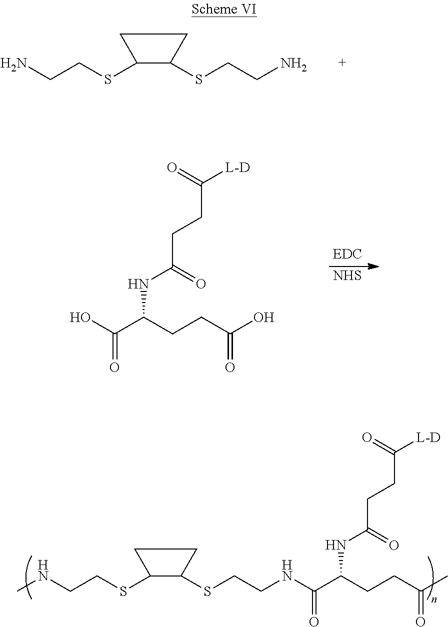

L-D in Scheme VI is as described in Scheme IV Scheme VI, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

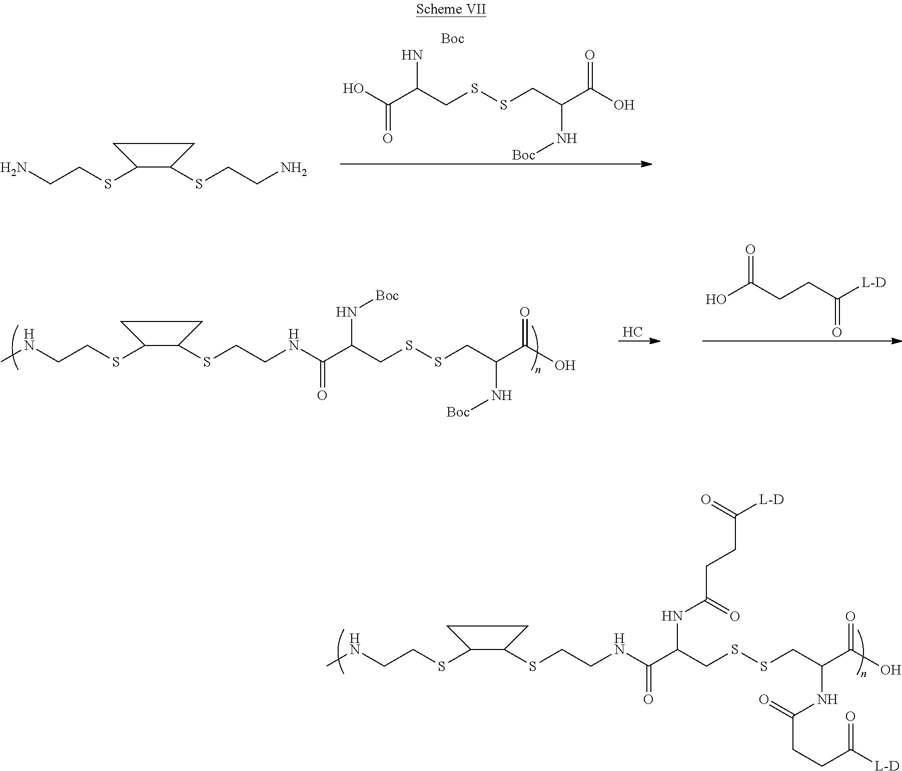

L-D in Scheme VII is as described in Scheme IV. Scheme VII, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

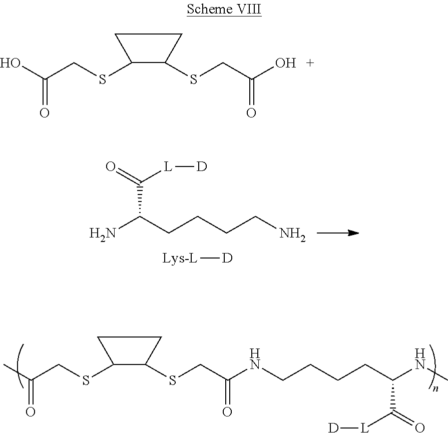

L-D in Scheme VIII is as described in Scheme IV. Scheme VIII, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

Additional examples of methods of synthesizing CDP-boronic acid conjugates are shown in Schemes IX-XIV below. L-D in Schemes IX-XIV are as described in Scheme IV. In each of Schemes IX-XIV, one or more of the L-D moieties in the CDP-boronic acid conjugate can be replaced with another therapeutic agent, e.g., another anticancer agent or anti-inflammatory agent, along with an optional linker.

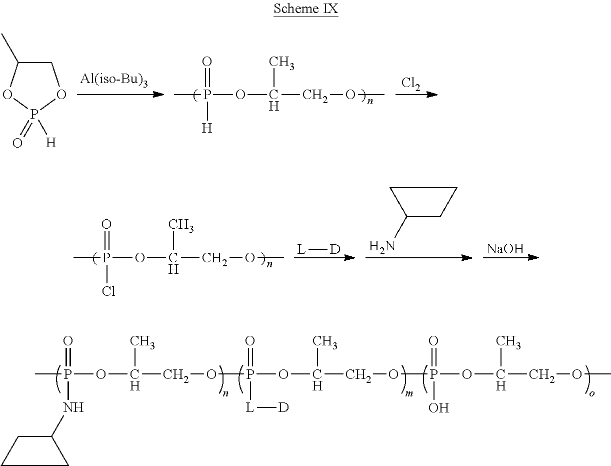

Scheme IX, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

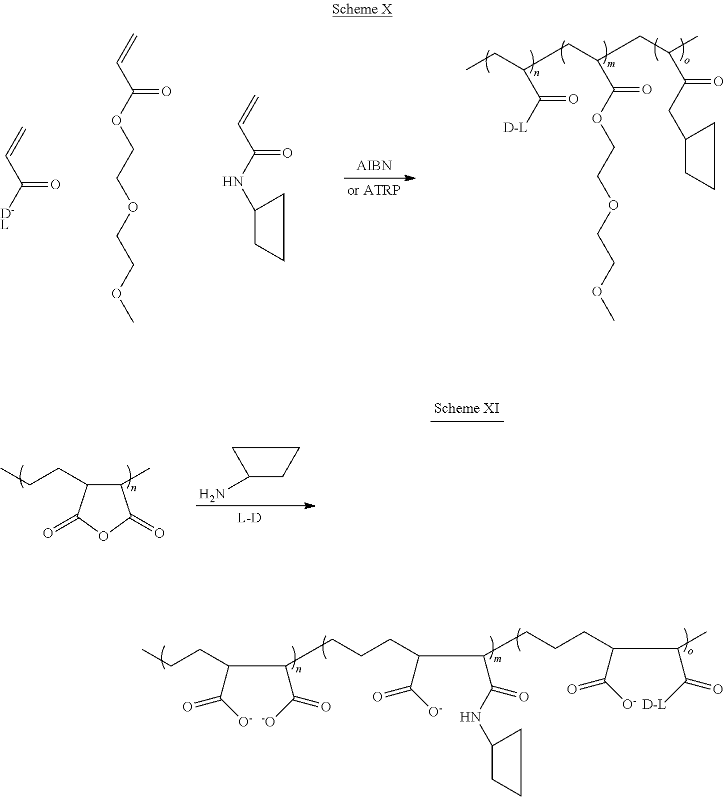

Scheme XI, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

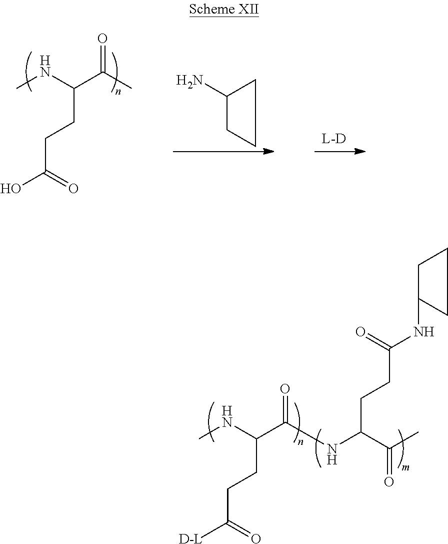

Scheme XII, as provided above, includes embodiments where boronic acid is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

The present invention further contemplates CDPs and CDP-conjugates synthesized using CD-bicysteine monomer and a di-NHS ester such as PEG-DiSPA or PEG-BTC as shown in Schemes XIII-XIV below.

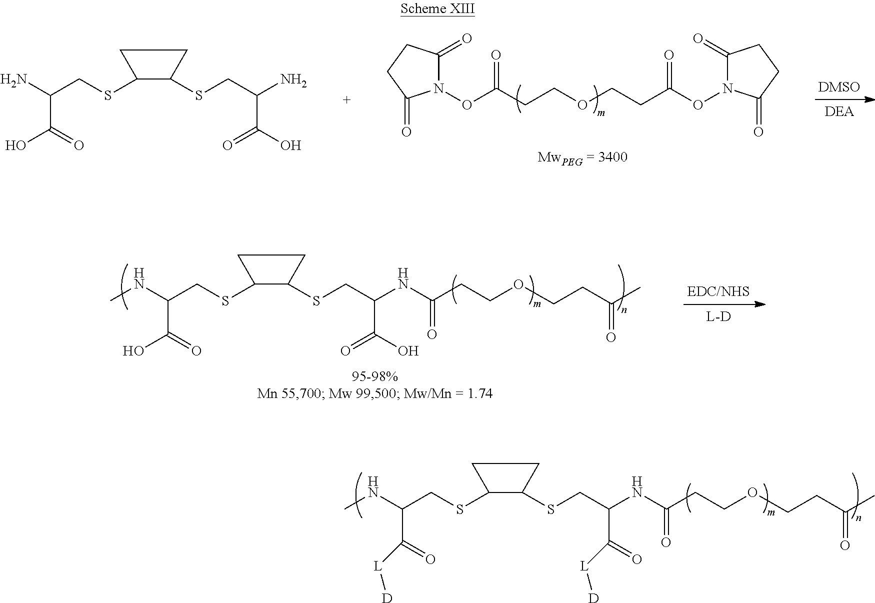

Scheme XIII, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

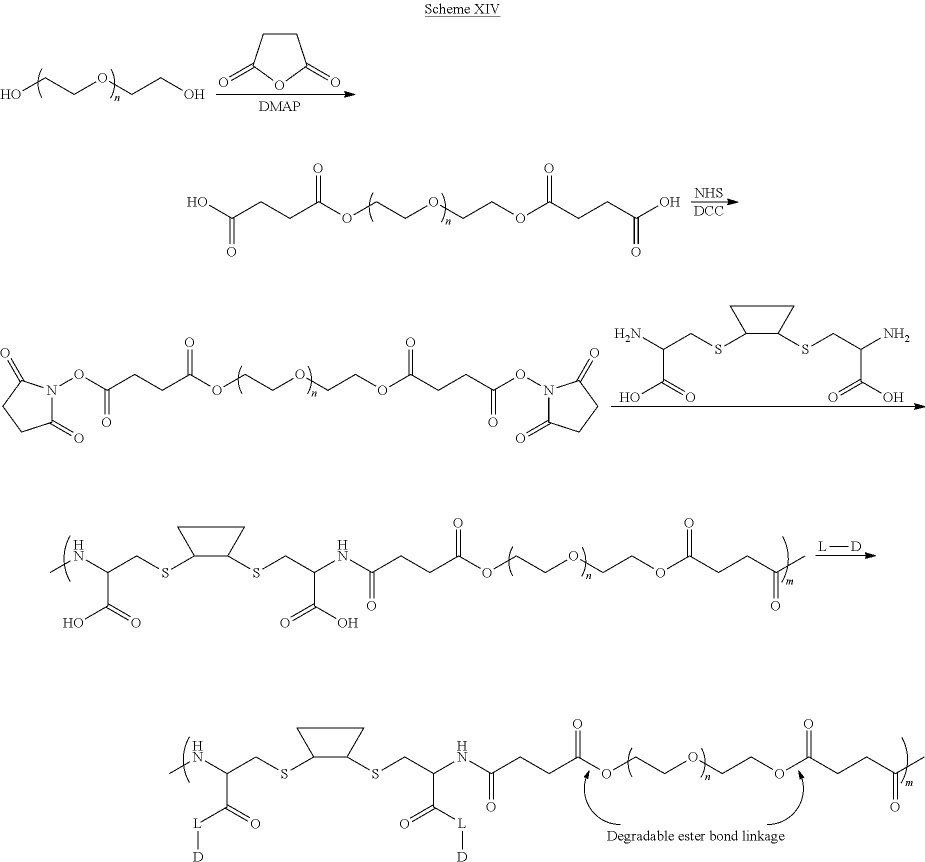

Scheme XIV, as provided above, includes embodiments where L-D is absent in one or more positions as provided above. This can be achieved, for example, when less than 100% yield is achieved when coupling the L-D to the polymer and/or when less than an equivalent amount of L-D is used in the reaction. Accordingly, the loading of the boronic acid, by weight of the polymer, can vary.

In some embodiments, a CDP-proteasome inhibitor (such as a boronic acid containing proteasome inhibitor) conjugate, e.g., a CDP-bortezomib conjugate, can be made by providing a CDP comprising cyclodextrin moieties and comonomers which do not contain cyclodextrin moieties (comonomers), wherein the cyclodextrin moieties and comonomers alternate in the CDP and wherein the CDP comprises at least four, five, six, seven, eight, etc. cyclodextrin moieties and at least four, five, six, seven, eight, etc. comonomers; and attaching a proteasome inhibitor (such as a boronic acid containing proteasome inhibitor, e.g., bortezomib) to the CDP.

In some embodiments, one or more of the proteasome inhibitors (such as a boronic acid containing proteasome inhibitors, e.g., bortezomib) moieties in the CDP-proteasome inhibitor (such as a boronic acid containing proteasome inhibitor) conjugate, e.g., a CDP-bortezomib conjugate, can be replaced with another therapeutic agent, e.g., another anticancer agent or anti-inflammatory agent.

In some embodiments, the proteasome inhibitor (such as a boronic acid containing proteasome inhibitors, e.g., bortezomib) is attached via a linker. In some embodiments, the proteasome inhibitor (such as a boronic acid containing proteasome inhibitors, e.g., bortezomib) is attached to the water soluble linear polymer through an attachment that is cleaved under biological conditions to release the proteasome inhibitor (such as a boronic acid containing proteasome inhibitors, e.g., bortezomib). In some embodiments, the proteasome inhibitor (such as a boronic acid containing proteasome inhibitors, e.g., bortezomib) is attached to the water soluble linear polymer at a cyclodextrin moiety or a comonomer. In some embodiments, the proteasome inhibitor (such as a boronic acid containing proteasome inhibitors, e.g., bortezomib) is attached to the water soluble linear polymer via an optional linker to a cyclodextrin moiety or a comonomer.

In some embodiments, the cyclodextrin moieties comprise linkers to which therapeutic agents are linked. In some embodiments, the cyclodextrin moieties comprise linkers to which therapeutic agents are linked via a second linker.

In some embodiments, the CDP is made by a process comprising: providing cyclodextrin moiety precursors, providing comonomer precursors, and copolymerizing said cyclodextrin moiety precursors and comonomer precursors to thereby make a CDP comprising cyclodextrin moieties and comonomers. In some embodiments, the CDP is conjugated with a proteasome inhibitor (such as a boronic acid containing proteasome inhibitors, e.g., bortezomib) to provide a CDP-proteasome inhibitor conjugate.

In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the proteasome inhibitor (such as a boronic acid containing proteasome inhibitors, e.g., bortezomib) is attached to the CDP via a linker. In some embodiments, the linker is cleaved under biological conditions.

In some embodiments, the proteasome inhibitor (such as a boronic acid containing proteasome inhibitors, e.g., bortezomib) makes up at least 5%, 10%, 15%, 20%, 25%, 30%, or even 35% by weight of the CDP-proteasome inhibitor conjugate.

In some embodiments, the comonomer comprises polyethylene glycol of molecular weight 3,400 Da, the cyclodextrin moiety comprises beta-cyclodextrin, the theoretical maximum loading of proteasome inhibitor (such as a boronic acid containing proteasome inhibitors, e.g., bortezomib) on the CDP-proteasome inhibitor conjugate is 13%, and proteasome inhibitor is 6-10% by weight of the CDP-proteasome inhibitor conjugate.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent is achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring. In some embodiments, the cyclodextrin moiety comprises an alpha, beta, or gamma cyclodextrin moiety.

In some embodiments, the proteasome inhibitor (such as a boronic acid containing proteasome inhibitors, e.g., bortezomib) is poorly soluble in water.

In some embodiments, the solubility of the proteasome inhibitor is <5 mg/ml at physiological pH.

In some embodiments, the proteasome inhibitor (such as a boronic acid containing proteasome inhibitors, e.g., bortezomib) is a hydrophobic compound with a log P>0.4, >0.6, >0.8, >1, >2, >3, >4, or >5. In some embodiments, the proteasome inhibitor (such as a boronic acid containing proteasome inhibitors, e.g., bortezomib) is hydrophobic and is attached via a second compound.

In some embodiments, administration of the CDP-proteasome inhibitor (such as a boronic acid containing proteasome inhibitors) conjugate, e.g., a CDP-bortezomib conjugate, to a subject results in release of the proteasome inhibitor over a period of at least 6 hours. In some embodiments, administration of the CDP-proteasome inhibitor (such as a boronic acid containing proteasome inhibitors) conjugate, e.g., a CDP-bortezomib conjugate to a subject results in release of the proteasome inhibitor over a period of 6 hours to a month. In some embodiments, upon administration of the CDP-proteasome inhibitor (such as a boronic acid containing proteasome inhibitors) conjugate, e.g., a CDP-bortezomib conjugate, to a subject the rate of proteasome inhibitor release is dependent primarily upon the rate of hydrolysis as opposed to enzymatic cleavage.

In some embodiments, the CDP-proteasome inhibitor (such as a boronic acid containing proteasome inhibitors) conjugate, e.g., a CDP-bortezomib conjugate, has a molecular weight of 10,000-500,000.

In some embodiments, the cyclodextrin moieties make up at least about 2%, 5%, 10%, 20%, 30%, 50% or 80% of the polymer by weight.

In some embodiments, a the CDP includes a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, a comonomer comprises a polyglycolic acid or polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In some embodiments, a CDP-polymer conjugate of the following formula can be made as follows:

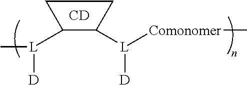

providing a polymer of the formula below:

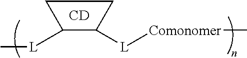

and coupling the polymer with a plurality of D moieties, wherein each D is independently absent or a proteasome inhibitor attached an optional linker, to provide:

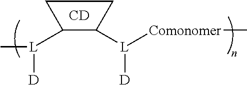

wherein the comonomer has a Mw of 2000 to 5000 Da (e.g., 3000 to 4000 Da, e.g., about 3.4 kDa) and n is at least 4, 5, 6, 7, 8, 9, 10, 11, 12, 13, 14, 15, 16, 17, 18, 19 or 20.

In some embodiments, one or more of the proteasome inhibitor moieties in the CDP-proteasome inhibitor conjugate can be replaced with another therapeutic agent, e.g., another anticancer agent or anti-inflammatory agent.

In some embodiments, a CDP-polymer conjugate of the following formula can be made as follows:

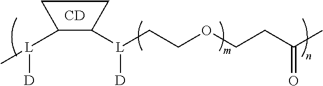

providing a polymer of the formula below:

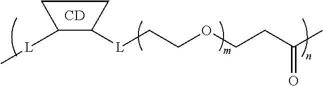

and coupling the polymer with a plurality of D moieties, wherein each D is independently absent or a proteasome inhibitor attached to an optional linker, to provide:

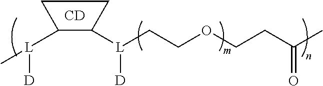

wherein the group

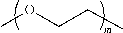

has a Mw of 3.4 kDa or less and n is at least

4, 5, 6, 7, 8, 9, 10, 11, 12, 13, 14, 15, 16, 17, 18, 19 or 20.

In some embodiments, one or more of the proteasome inhibitor moieties in the CDP-proteasome inhibitor conjugate can be replaced with another therapeutic agent, e.g., another anticancer agent or anti-inflammatory agent.

The reaction scheme as provided above includes embodiments where D is absent in one or more positions as provided above. This can be achieved, for example, when less than 100% yield is achieved when coupling the proteasome inhibitor to the polymer and/or when less than an equivalent amount of proteasome inhibitor is used in the reaction. Accordingly, the loading of the proteasome inhibitor, by weight of the polymer, can vary, for example, the loading of the proteasome inhibitor can be at least about 3% by weight, e.g., at least about 55, at least about 8%, at least about 10%, at least about 13%, at least about 15%, or at least about 20%.

In some embodiments, a CDP-proteasome inhibitor conjugate of the following formula can be made as follows:

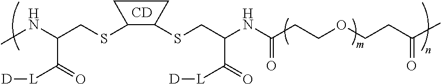

providing a polymer below:

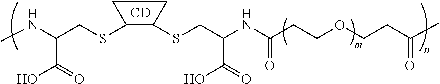

and coupling the polymer with a plurality of L-D moieties, wherein L is a linker or absent and D is a proteasome inhibitor, to provide:

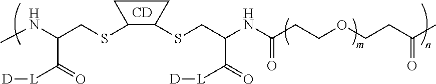

wherein the group

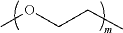

has a Mw of 3.4 kDa or less and n is at least

4, 5, 6, 7, 8, 9, 10, 11, 12, 13, 14, 15, 16, 17, 18, 19 or 20.

In some embodiments, one or more of the proteasome inhibitor moieties in the CDP-proteasome inhibitor conjugate can be replaced with another therapeutic agent, e.g., another anticancer agent or anti-inflammatory agent.

The reaction scheme as provided above includes embodiments where L-D is absent in one or more positions as provided above. This can be achieved, for example, when less than 100% yield is achieved when coupling the proteasome inhibitor-linker to the polymer and/or when less than an equivalent amount of proteasome inhibitor-linker is used in the reaction. Accordingly, the loading of the proteasome inhibitor, by weight of the polymer, can vary, for example, the loading of the proteasome inhibitor can be at least about 3% by weight, e.g., at least about 5%, at least about 8%, at least about 10%, at least about 13%, at least about 15%, or at least about 20%.

In some embodiments, at least a portion of the L moieties of L-D is absent. In some embodiments, each L is independently an amino acid or derivative thereof (e.g., glycine).

In some embodiments, the coupling of the polymer with the plurality of L-D moieties results in the formation of a plurality of amide bonds.

In certain instances, the CDPs are random copolymers, in which the different subunits and/or other monomeric units are distributed randomly throughout the polymer chain. Thus, where the formula Xₘ—Yₙ—Zₒ appears, wherein X, Y and Z are polymer subunits, these subunits may be randomly interspersed throughout the polymer backbone. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing, or treatment.

Pharmaceutical Compositions

In another aspect, the present invention provides a composition, e.g., a pharmaceutical composition, comprising a CDP-proteasome inhibitor conjugate and a pharmaceutically acceptable carrier or adjuvant.

In some embodiments, a pharmaceutical composition may include a pharmaceutically acceptable salt of a compound described herein, e.g., a CDP-proteasome inhibitor (such as a boronic acid containing proteasome inhibitors) conjugate, e.g., a CDP-bortezomib conjugate. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a CDP-proteasome inhibitor (such as a boronic acid containing proteasome inhibitors) conjugate, e.g., a CDP-bortezomib conjugate, which may be any liquid solution compatible with the CDP-proteasome inhibitor (such as a boronic acid containing proteasome inhibitors) conjugate, e.g., a CDP-bortezomib conjugate, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, and mixtures thereof.

A composition described herein may also include another component, such as an antioxidant, antibacterial, buffer, bulking agent, chelating agent, an inert gas, a tonicity agent and/or a viscosity agent.

In one embodiment, the CDP-proteasome inhibitor (such as a boronic acid containing proteasome inhibitors) conjugate, e.g., a CDP-bortezomib conjugate is provided in lyophilized form and is reconstituted prior to administration to a subject. The lyophilized CDP-proteasome inhibitor (such as a boronic acid containing proteasome inhibitors) conjugate, e.g., a CDP-bortezomib conjugate can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the CDP-proteasome inhibitor (such as a boronic acid containing proteasome inhibitors) conjugate, e.g., a CDP-bortezomib conjugate from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxomers, Tweens, lecithins, saccharides, oligosaccharides, polysaccharides and polyols (e.g., trehalose, mannitol, sorbitol, lactose, sucrose, glucose and dextran), salts and crown ethers.

In some embodiments, the lyophilized CDP-proteasome inhibitor (such as a boronic acid containing proteasome inhibitors) conjugate, e.g., a CDP-bortezomib conjugate, is reconstituted with a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution having a concentration of about 2 mg/mL to about 4 mg/mL of the CDP-proteasome inhibitor (such as a boronic acid containing proteasome inhibitors) conjugate, e.g., a CDP-bortezomib conjugate. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject CDP-proteasome inhibitor (such as a boronic acid containing proteasome inhibitors) conjugate, e.g., a CDP-bortezomib conjugate, with a third vial containing sufficient parenteral diluent to prepare the final concentration for administration. A typical diluent is Lactated Ringer's Injection.

The final dilution of the reconstituted CDP-proteasome inhibitor (such as a boronic acid containing proteasome inhibitors) conjugate, e.g., a CDP-bortezomib conjugate may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

### Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional or intracranial injection), topically, mucosally (e.g., rectally or vaginally), nasally, buccally, ophthalmically, via inhalation spray (e.g., delivered via nodulation, propellant or a dry powder device) or via an implanted reservoir.

Pharmaceutical compositions suitable for parenteral administration comprise one or more CDP-proteasome inhibitor (such as a boronic acid containing proteasome inhibitors) conjugate(s), e.g., a CDP-bortezomib conjugate, in combination with one or more pharmaceutically acceptable sterile isotonic aqueous or no aqueous solutions, dispersions, suspensions or emulsions, or sterile powders which may be reconstituted into sterile inject able solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the CDP-proteasome inhibitor (such as a boronic acid containing proteasome inhibitors) conjugate, e.g., a CDP-bortezomib conjugate then depends upon its rate of dissolution which, in turn, may depend upon crystal size and crystalline form. Alternatively, delayed absorption of a parenterally administered drug form is accomplished by dissolving or suspending the CDP-proteasome inhibitor (such as a boronic acid containing proteasome inhibitors) conjugate, e.g., a CDP-bortezomib conjugate in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the CDP-proteasome inhibitor (such as a boronic acid containing proteasome inhibitors) conjugate, e.g., a CDP-bortezomib conjugate, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the CDP-proteasome inhibitor (such as a boronic acid containing proteasome inhibitors) conjugate, e.g., a CDP-bortezomib conjugate, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle (e.g., nanoparticle) described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle (e.g., nanoparticle) suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more CDP-proteasome inhibitor (such as a boronic acid containing proteasome inhibitors) conjugates, e.g., one or more CDP-bortezomib conjugates, described herein with one or more suitable non-irritating excipients which is solid at room temperature, but liquid at body temperature. The composition will therefore melt in the rectum or vaginal cavity and release the CDP-proteasome inhibitor (such as a boronic acid containing proteasome inhibitors) conjugate, e.g., a CDP-bortezomib conjugate. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention.

Dosages and Dosage Regimens

The CDP-proteasome inhibitor (such as a boronic acid containing proteasome inhibitors) conjugate, e.g., a CDP-bortezomib conjugate can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the CDP-proteasome inhibitor (such as a boronic acid containing proteasome inhibitors) conjugate, e.g., a CDP-bortezomib conjugate is administered to a subject at a dosage of, e.g., about 0.1 to 30 m g/m², about 0.1-5 mg/m2, about 0.5-3 mg/m², e.g., about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mg/m² of the proteasome inhibitor. Administration can be at regular intervals, such as every 1, 2, 3, 4, or 5 days, or weekly, or every 2, 3, 4, 5, 6, or 7 or 8 weeks. The administration can be over a period of from about 0-10 minutes, e.g. from about 0.1 second to 5 minutes, from about 0.1 second to 60 seconds, from about 1 second to 30 seconds, from about 2 seconds to 10 seconds and from about 3 seconds to 5 seconds. In one embodiment, the CDP-proteasome inhibitor conjugate is administered as a bolus infusion or intravenous injection, e.g., over a period of 30 seconds, 10 seconds, 5 seconds, 4 seconds, 3 seconds, 2 seconds or 1 second. In one embodiment, the CDP-proteasome inhibitor (such as a boronic acid containing proteasome inhibitors) conjugate, e.g., a CDP-bortezomib conjugate is administered in an amount such the desired dose of the agent is administered. Preferably the dose of the CDP-proteasome inhibitor (such as a boronic acid containing proteasome inhibitors) conjugate, e.g., a CDP-bortezomib conjugate is a dose described herein.

In one embodiment, the subject receives 1, 2, 3, up to 15 treatments, or more, or until the disorder or a symptom of the disorder is cured, healed, alleviated, relieved, altered, remedied, ameliorated, palliated, improved or affected. For example, the subject receive an intravenous injection twice a week until the disorder or a symptom of the disorder are cured, healed, alleviated, relieved, altered, remedied, ameliorated, palliated, improved or affected. Preferably, the dosing schedule is a dosing schedule described herein.

The CDP-proteasome inhibitor (such as a boronic acid containing proteasome inhibitors) conjugate, e.g., a CDP-bortezomib conjugate, can be administered as a first line therapy, e.g., alone or in combination with an additional agent or agents. In other embodiments, a CDP-proteasome inhibitor (such as a boronic acid containing proteasome inhibitors) conjugate, e.g., a CDP-bortezomib conjugate, is administered after a subject has developed resistance to, has failed to respond to or has relapsed after a first line therapy. The CDP-proteasome inhibitor (such as a boronic acid containing proteasome inhibitors) conjugate, e.g., a CDP-bortezomib conjugate, can be administered in combination with a second agent. Preferably, the CDP-proteasome inhibitor (such as a boronic acid containing proteasome inhibitors) conjugate, e.g., a CDP-bortezomib conjugate, is administered in combination with a second agent described herein.

### Kits

A CDP-proteasome inhibitor (such as a boronic acid containing proteasome inhibitors) conjugate, e.g., a CDP-bortezomib conjugate described herein may be provided in a kit. The kit includes a CDP-proteasome inhibitor (such as a boronic acid containing proteasome inhibitors) conjugate, e.g., a CDP-bortezomib conjugate described herein and, optionally, a container, a pharmaceutically acceptable carrier and/or informational material. The informational material can be descriptive, instructional, marketing or other material that relates to the methods described herein and/or the use of the CDP-proteasome inhibitor (such as a boronic acid containing proteasome inhibitors) conjugate, e.g., a CDP-bortezomib conjugate, for the methods described herein.

The informational material of the kits is not limited in its form. In one embodiment, the informational material can include information about production of the CDP-proteasome inhibitor (such as a boronic acid containing proteasome inhibitors) conjugate, e.g., a CDP-bortezomib conjugate, physical properties of the CDP-proteasome inhibitor (such as a boronic acid containing proteasome inhibitors) conjugate, e.g., a CDP-bortezomib conjugate, concentration, date of expiration, batch or production site information, and so forth. In one embodiment, the informational material relates to methods for administering the CDP-proteasome inhibitor (such as a boronic acid containing proteasome inhibitors) conjugate, e.g., a CDP-bortezomib conjugate.

In one embodiment, the informational material can include instructions to administer a CDP-proteasome inhibitor (such as a boronic acid containing proteasome inhibitors) conjugate, e.g., a CDP-bortezomib conjugate, described herein in a suitable manner to perform the methods described herein, e.g., in a suitable dose, dosage form, or mode of administration (e.g., a dose, dosage form, or mode of administration described herein). In another embodiment, the informational material can include instructions to administer a CDP-proteasome inhibitor (such as a boronic acid containing proteasome inhibitors) conjugate, e.g., a CDP-bortezomib conjugate, described herein to a suitable subject, e.g., a human, e.g., a human having or at risk for a disorder described herein. In another embodiment, the informational material can include instructions to reconstitute a CDP-proteasome inhibitor conjugate described herein into a pharmaceutically acceptable composition.

In one embodiment, the kit includes instructions to use the CDP-proteasome inhibitor (such as a boronic acid containing proteasome inhibitors) conjugate, e.g., a CDP-bortezomib conjugate, such as for treatment of a subject. The instructions can include methods for reconstituting or diluting the CDP-proteasome inhibitor (such as a boronic acid containing proteasome inhibitors) conjugate, e.g., a CDP-bortezomib conjugate, for use with a particular subject or in combination with a particular chemotherapeutic agent. The instructions can also include methods for reconstituting or diluting the CDP-proteasome inhibitor (such as a boronic acid containing proteasome inhibitors) conjugate, e.g., a CDP-bortezomib conjugate, for use with a particular means of administration, such as by intravenous injection.

In another embodiment, the kit includes instructions for treating a subject with a particular indication, such as a particular cancer, or a cancer at a particular stage. For example, the instructions can be for a cancer or cancer at stage described herein. The instructions may also address first line treatment of a subject who has a particular cancer, or cancer at a stage described herein. The instructions can also address treatment of a subject who has been non-responsive to a first line therapy or has become sensitive (e.g., has one or more unacceptable side effect) to a first line therapy, such as a taxane, an anthracycline, an alkylating agent, a platinum based agent, a vinca alkaloid. In another embodiment, the instructions will describe treatment of selected subjects with the CDP-proteasome inhibitor (such as a boronic acid containing proteasome inhibitors) conjugate, e.g., a CDP-bortezomib conjugate. For example, the instructions can describe treatment of one or more of: a subject who has received an anticancer agent (e.g., a bortezomib) and has a neutrophil and/or platelet count less than a standard; a subject who has moderate to severe neutropenia; a subject who has experienced one or more symptom of neuropathy from treatment with an anticancer agent, e.g., bortezomib, a taxane, a vinca alkaloid, an alkylating agent, an anthracycline, a platinum-based agent or an epothilone; a subject who has experienced hypotension or has or is at risk for having hypotension to treatment with an anticancer agent (e.g., bortezomib); a subject who has experienced a cardiac disorder or has or is at risk for having a cardiac disorder to treatment with an anticancer agent (e.g., bortezomib); a subject who has experienced a pulmonary disorder or has or is at risk for having a pulmonary disorder to treatment with an anticancer agent (e.g., bortezomib); a subject who has experienced RPLS or has or is at risk for having RPLS to treatment with an anticancer agent (e.g., bortezomib); and a subject who has experienced gastrointestinal adverse events or has or is at risk for having gastrointestinal adverse events to treatment with an anticancer agent (e.g., bortezomib); a.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a CDP-proteasome inhibitor (such as a boronic acid containing proteasome inhibitors) conjugate, e.g., a CDP-bortezomib conjugate, described herein and/or its use in the methods described herein. The informational material can also be provided in any combination of formats.

In addition to a CDP-proteasome inhibitor (such as a boronic acid containing proteasome inhibitors) conjugate, e.g., a CDP-bortezomib conjugate, described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a CDP-proteasome inhibitor (such as a boronic acid containing proteasome inhibitors) conjugate, e.g., a CDP-bortezomib conjugate, described herein. In such embodiments, the kit can include instructions for admixing a CDP-proteasome inhibitor (such as a boronic acid containing proteasome inhibitors) conjugate, e.g., a CDP-bortezomib conjugate, described herein and the other ingredients, or for using a CDP-proteasome inhibitor (such as a boronic acid containing proteasome inhibitors) conjugate, e.g., a CDP-bortezomib conjugate described herein together with the other ingredients.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the CDP-proteasome inhibitor (such as a boronic acid containing proteasome inhibitors) conjugate, e.g., a CDP-bortezomib conjugate, and the second therapeutic agent are in separate containers, and in another embodiment, the CDP-proteasome inhibitor (such as a boronic acid containing proteasome inhibitors) conjugate, e.g., a CDP-bortezomib conjugate, and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A CDP-proteasome inhibitor (such as a boronic acid containing proteasome inhibitors) conjugate, e.g., a CDP-bortezomib conjugate, described herein can be provided in any form, e.g., liquid, frozen, dried or lyophilized form. It is preferred that a particle (e.g., nanoparticle) described herein be substantially pure and/or sterile. When a CDP-proteasome inhibitor (such as a boronic acid containing proteasome inhibitors) conjugate, e.g., a CDP-bortezomib conjugate, described herein is provided in a liquid solution, the liquid solution preferably is an aqueous solution, with a sterile aqueous solution being preferred. In one embodiment, the CDP-proteasome inhibitor (such as a boronic acid containing proteasome inhibitors) conjugate, e.g., a CDP-bortezomib conjugate is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a CDP-proteasome inhibitor (such as a boronic acid containing proteasome inhibitors) conjugate, e.g., a CDP-bortezomib conjugate, described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a CDP-proteasome inhibitor (such as a boronic acid containing proteasome inhibitors) conjugate, e.g., a CDP-bortezomib conjugate, described herein. For example, the kit includes a plurality of syringes, ampoules, foil packets, or blister packs, each containing a single unit dose of a particle (e.g., nanoparticle) described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

Combination Therapy

The CDP-proteasome inhibitor (such as a boronic acid containing proteasome inhibitors) conjugate, e.g., a CDP-bortezomib conjugate, may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The CDP-proteasome inhibitor (such as a boronic acid containing proteasome inhibitors) conjugate, e.g., a CDP-bortezomib conjugate, and the at least one additional therapeutic agent can be administered simultaneously, in the same or in separate compositions, or sequentially. For sequential administration, the CDP-proteasome inhibitor (such as a boronic acid containing proteasome inhibitors) conjugate, e.g., a CDP-bortezomib conjugate, can be administered first, and the additional agent can be administered second, or the order of administration can be reversed.

In some embodiments, the CDP-proteasome inhibitor (such as a boronic acid containing proteasome inhibitors) conjugate, e.g., a CDP-bortezomib conjugate, is administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the CDP-proteasome inhibitor (such as a boronic acid containing proteasome inhibitors) conjugate, e.g., a CDP-bortezomib conjugate is administered with at least one additional therapeutic agent, such as a chemotherapeutic agent. In certain embodiments, the CDP-proteasome inhibitor (such as a boronic acid containing proteasome inhibitors) conjugate, e.g., a CDP-bortezomib conjugate is administered in combination with one or more additional chemotherapeutic agent, e.g., with one or more chemotherapeutic agents described herein. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®), larotaxel, cabazitaxel.

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

proteosome inhibitors: bortezomib (Velcade®).

interferons (e.g., IFN-alpha (Alferon®, Roferon-A®, Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3/(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9,1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

proteasome inhibitors which include, without limitation aclacinomycin A, gliotoxin and bortezomib (Velcade®) or any proteasome inhibitor described herein.

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives of formula I as generically and specifically described in PCT Publication No.: WO 03/013541 and U.S. Publication No.: 2008/0139587:

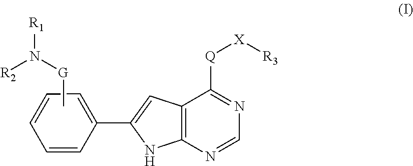

Publication No.: 2008/0139587 discloses the various substituents, e.g., R₁, R₂, etc.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™)

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiments, the CDP-proteasome inhibitor (such as a boronic acid containing proteasome inhibitors) conjugate, e.g., a CDP-bortezomib conjugate, is administered instead of a microtubule affecting agent, e.g., instead of a microtubule affecting agent as a first line therapy or a second line therapy. For example, the CDP-proteasome inhibitor (such as a boronic acid containing proteasome inhibitors) conjugate, e.g., a CDP-bortezomib conjugate, can be used instead of any of the following microtubule affecting agents allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), taxol derivatives (e.g., derivatives (e.g., NSC 608832), thiocolchicine (NSC 361792), trityl cysteine (NSC 83265), vinblastine sulfate (NSC 49842), vincristine sulfate (NSC 67574).

In some cases, a hormone and/or steriod can be administered in combination with a CDP-proteasome inhibitor (such as a boronic acid containing proteasome inhibitors) conjugate, e.g., a CDP-bortezomib conjugate. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the CDP-proteasome inhibitor (such as a boronic acid containing proteasome inhibitors) conjugate, e.g., a CDP-bortezomib conjugate, is administered in combination with an anti-microbial (e.g., leptomycin B).

In another embodiment, the CDP-proteasome inhibitor (such as a boronic acid containing proteasome inhibitors) conjugate, e.g., a CDP-bortezomib conjugate, is administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IRO), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present invention are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 1730, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the CDP-proteasome inhibitor (such as a boronic acid containing proteasome inhibitors) conjugate, e.g., a CDP-bortezomib conjugate, is administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), cacineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some embodiments, a CDP-proteasome inhibitor (such as a boronic acid containing proteasome inhibitors) conjugate, e.g., a CDP-bortezomib conjugate is administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

When formulating the pharmaceutical compositions featured in the invention the clinician may utilize preferred dosages as warranted by the condition of the subject being treated. For example, in one embodiment, a CDP-proteasome inhibitor (such as a boronic acid containing proteasome inhibitors) conjugate, e.g., a CDP-bortezomib conjugate, may be administered at a dosing schedule described herein, e.g., once every one, two three four, five, or six weeks.

Also, in general, a CDP-proteasome inhibitor (such as a boronic acid containing proteasome inhibitors) conjugate, e.g., a CDP-bortezomib conjugate, and an additional chemotherapeutic agent(s) do not have to be administered in the same pharmaceutical composition, and may, because of different physical and chemical characteristics, have to be administered by different routes. For example, the CDP-proteasome inhibitor (such as a boronic acid containing proteasome inhibitors) conjugate, e.g., a CDP-bortezomib conjugate, may be administered intravenously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The actual dosage of the CDP-proteasome inhibitor (such as a boronic acid containing proteasome inhibitors) conjugate, e.g., a CDP-bortezomib conjugate, and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In some embodiments, when a CDP-proteasome inhibitor (such as a boronic acid containing proteasome inhibitors) conjugate, e.g., a CDP-bortezomib conjugate, is administered in combination with one or more additional chemotherapeutic agent, the additional chemotherapeutic agent (or agents) is administered at a standard dose. For example, a standard dosage for melphalan is 9 mg/m²; and a standard dosage for prednisone is 60 mg/m².

The disclosure also encompasses a method for the synergistic treatment of cancer wherein a CDP-proteasome inhibitor (such as a boronic acid containing proteasome inhibitors) conjugate, e.g., a CDP-bortezomib conjugate, is administered in combination with an additional chemotherapeutic agent or agents.

The particular choice of conjugate and anti-proliferative cytotoxic agent(s) or radiation will depend upon the diagnosis of the attending physicians and their judgment of the condition of the subject and the appropriate treatment protocol.

If the CDP-proteasome inhibitor (such as a boronic acid containing proteasome inhibitors) conjugate, e.g., a CDP-bortezomib conjugate, and the chemotherapeutic agent(s) and/or radiation are not administered simultaneously or essentially simultaneously, then the initial order of administration of the CDP-proteasome inhibitor (such as a boronic acid containing proteasome inhibitors) conjugate, e.g., a CDP-bortezomib conjugate, and the chemotherapeutic agent(s) and/or radiation, may be varied. Thus, for example, the CDP-proteasome inhibitor (such as a boronic acid containing proteasome inhibitors) conjugate, e.g., a CDP-bortezomib conjugate, may be administered first followed by the administration of the chemotherapeutic agent(s) and/or radiation; or the chemotherapeutic agent(s) and/or radiation may be administered first followed by the administration of the CDP-proteasome inhibitor (such as a boronic acid containing proteasome inhibitors) conjugate, e.g., a CDP-bortezomib conjugate. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (CDP-proteasome inhibitor conjugate, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Indications

The disclosed CDP-proteasome inhibitor (such as a boronic acid containing proteasome inhibitors) conjugates, e.g., a CDP-bortezomib conjugate, are useful in evaluating or treating proliferative disorders, e.g., treating a tumor and metastases thereof wherein the tumor or metastases thereof is a cancer described herein. The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer, childhood; esophageal cancer; esophageal cancer, childhood; gallbladder cancer; gastric (stomach) cancer; gastric (stomach) cancer, childhood; hepatocellular (liver) cancer, adult (primary); hepatocellular (liver) cancer, childhood (primary); extrahepatic; pancreatic cancer; pancreatic cancer, childhood; sarcoma, rhabdomyosarcoma; pancreatic cancer, islet cell; rectal cancer; and small intestine cancer;

Endocrine cancers such as islet cell carcinoma (endocrine pancreas); adrenocortical carcinoma; adrenocortical carcinoma, childhood; gastrointestinal carcinoid tumor; parathyroid cancer; pheochromocytoma; pituitary tumor; thyroid cancer; thyroid cancer, childhood; multiple endocrine neoplasia syndrome, childhood; and carcinoid tumor, childhood;

Eye cancers such as intraocular melanoma; and retinoblastoma;

Musculoskeletal cancers such as Ewing's family of tumors; osteosarcoma/malignant fibrous histiocytoma of the bone; rhabdomyosarcoma, childhood; soft tissue sarcoma, adult; soft tissue sarcoma, childhood; clear cell sarcoma of tendon sheaths; and uterine sarcoma;

Breast cancer such as breast cancer and pregnancy; breast cancer, childhood; and breast cancer, male;

Neurologic cancers such as brain stem glioma, childhood; brain tumor, adult; brain stem glioma, childhood; cerebellar astrocytoma, childhood; cerebral astrocytoma/malignant glioma, childhood; ependymoma, childhood; medulloblastoma, childhood; pineal and supratentorial primitive neuroectodermal tumors, childhood; visual pathway and hypothalamic glioma, childhood; other childhood brain cancers; adrenocortical carcinoma; central nervous system lymphoma, primary; cerebellar astrocytoma, childhood; neuroblastoma; craniopharyngioma; spinal cord tumors; central nervous system atypical teratoid/rhabdoid tumor; central nervous system embryonal tumors; andsupratentorial primitive neuroectodermal tumors, childhood and pituitary tumor;

Genitourinary cancers such as bladder cancer; bladder cancer, childhood; kidney cancer; ovarian cancer, childhood; ovarian epithelial cancer; ovarian low malignant potential tumor; penile cancer; prostate cancer; renal cell cancer, childhood; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as extracranial germ cell tumor, childhood; extragonadal germ cell tumor; ovarian germ cell tumor; and testicular cancer;

Head and neck cancers such as lip and oral cavity cancer; oral cancer, childhood; hypopharyngeal cancer; laryngeal cancer; laryngeal cancer, childhood; metastatic squamous neck cancer with occult primary; mouth cancer; nasal cavity and paranasal sinus cancer; nasopharyngeal cancer; nasopharyngeal cancer, childhood; oropharyngeal cancer; parathyroid cancer; pharyngeal cancer; salivary gland cancer; salivary gland cancer, childhood; throat cancer; and thyroid cancer;

Hematologic/blood cell cancers such as a leukemia (e.g., acute lymphoblastic leukemia, adult; acute lymphoblastic leukemia, childhood; acute myeloid leukemia, adult; acute myeloid leukemia, childhood; chronic lymphocytic leukemia; chronic myelogenous leukemia; and hairy cell leukemia); a lymphoma (e.g., AIDS-related lymphoma; cutaneous T-cell lymphoma; Hodgkin's lymphoma, adult; Hodgkin's lymphoma, childhood; Hodgkin's lymphoma during pregnancy; non-Hodgkin's lymphoma, adult; non-Hodgkin's lymphoma, childhood; non-Hodgkin's lymphoma during pregnancy; mycosis fungoides; sezary syndrome; T-cell lymphoma, cutaneous; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; thymoma, childhood; thymic carcinoma; bronchial adenomas/carcinoids; pleuropulmonary blastoma; non-small cell lung cancer; and small cell lung cancer;

Skin cancers such as Kaposi's sarcoma; Merkel cell carcinoma; melanoma; and skin cancer, childhood;

Other childhood cancers and cancers of unknown primary site;

and metastases of the aforementioned cancers can also be treated or prevented in accordance with the methods described herein.

The CDP-proteasome inhibitor (such as a boronic acid containing proteasome inhibitors) conjugates, e.g., CDP-bortezomib conjugates, described herein are particularly suited to treat multiple myeloma and mantle cell lymphoma.

In one embodiment, a method is provided for a combination treatment of a cancer, such as by treatment with a CDP-proteasome inhibitor (such as a boronic acid containing proteasome inhibitors) conjugate, e.g., a CDP-bortezomib conjugate, and a second therapeutic agent. Various combinations are described herein. The combination can reduce the development of tumors, reduces tumor burden, or produce tumor regression in a mammalian host.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

In all the relevant names and structures below, the terminology CDP_{0.5} indicates that up to 2 molecules of linker and/or linker conjugated to drug may be attached to each cyclodextrin unit of the CDP polymer with the number of cyclodextrin units determined by the overall MW of the CDP polymer.

### Example 1

### Synthesis of CDP Conjugate with (aminoethyl)(hydroxyethyl)amine based boronic acid—Conjugate of bortezomib with [(6-(CDP_{0.5}-carboxamidohexyl)-(2-methylaminoethyl)-(2-hydroxyethyl)]amine

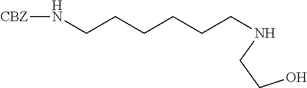

Step 1: (6-Benzyloxycarbonylaminohexyl)(2-hydroxyethyl)amine: In a manner similar to that described by Pellacini et al. (U.S. Pat. No. 6,455,576) the title compound will be prepared from 6-benzyloxycarbonylaminohexanol.

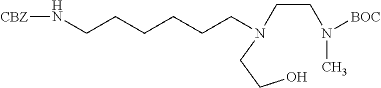

Step 2: (6-Benzyloxycarbonylaminohexyl)-((2-t-butloxycarbonyl)methylaminoethyl)-(2-hydroxyethyl)amine: In a manner similar to that described by Ackerman et al. (US Patent Appl. 2005065210) the title compound will be prepared from ((2-t-butoxycarbonyl)methylaminoethanol and (6-benzyloxycarbonylaminohexyl)(2-hydroxyethyl)amine (from Step 1).

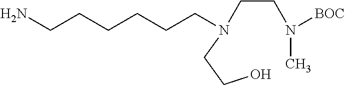

Step 3: (6-Aminohexyl)-((2-benzyloxycarbonyl)methylaminoethyl)-(2-hydroxyethyl)amine: (6-Benzyloxycarbonylaminohexyl)-((2-t-butoxycarbonyl)methylaminoethyl)-(2-hydroxyethyl)amine will be dissolved in MeOH (10 volumes). The mixture will stirred for 5 min to afford a clear solution. 5% Pd/C (200 mg, 50% moisture) will be charged. The flask will be evacuated for 1 min and then filled with H₂ with a balloon. The reaction will be stirred at ambient temperature for 3 h or until the reaction is complete. The structure will be confirmed with LC/MS and 1H-NMR.

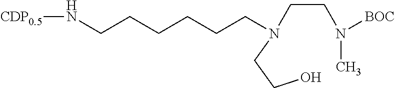

Step 4: (6-(CDP_{0.5}-carboxamidohexyl)-((2-t-butoxycarbonyl)methylaminoethyl)-(2-hydroxyethyl)amine: A 100-mL round-bottom flask will be charged with (6-aminohexyl)-((2-t-butoxycarbonyl)methylaminoethyl)-(2-hydroxyethyl)amine (2.0 mmol per estimated number of cyclodextrin units in the CDP polymer) and DMF (5 mL). The mixture will be stirred for 15 min to afford a clear solution. CDP (1 g) in DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

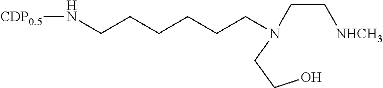

Step 5: (6-(CDP_{0.5}-carboxamidohexyl)-(methylaminoethyl)-(2-hydroxyethyl)amine: A round-bottom flask equipped with a magnetic stirrer will be charged with (6-(CDP_{0.5}-carboxamidohexyl)-((2-t-butoxycarbonyl)methylaminoethyl)-(2-hydroxyethyl)amine in CH₂Cl₂ (5 volumes). To this will be added TFA (5 volumes). The reaction will be stirred at ambient temperature for 3 h or until the reaction is complete. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

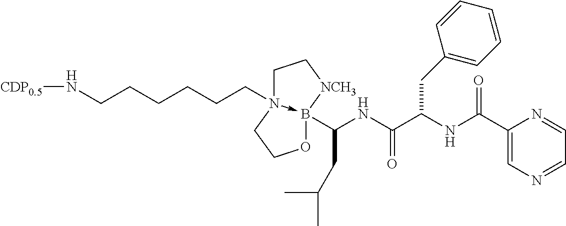

Step 6: Conjugate of bortezomib with (6-(CDP_{0.5}-carboxamidohexyl)-(methylaminoethyl)-(2-hydroxyethyl)amine: In a manner similar to that described by Hebel et al. (J. Org. Chem. 2002, 67, 9452) bortezomib (2.0 mmol per estimated number of cyclodextrin units in the CDP polymer) will be dissolved in DMF and treated with a solution of (6-(CDP_{0.5}-carboxamidohexyl)-(methylaminoethyl)-(2-hydroxyethyl)amine (1 g) in DMF and 4 Å MS. After 6 h at room temperature, the reaction mixture will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with ¹H-NMR, HPLC and GPC.

### Example 2

### Synthesis of CDP conjugate with 1,2-amino alcohol based boronic acid—Conjugate of bortezomib with (8-(CDP_{0.5}-carboxamido)-2-hydroxy-2-methyl-1-methylaminooctane

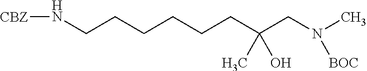

Step 1: (8-(benzyloxycarbonylamino)-2-hydroxy-2-methyl-1-((t-butoxycarbonyl)methylamino)octane: In the manner described by Ortiz et al. (Tetrahedron 1999, 55, 4831) the title compound will be prepared from 8-benzyloxycarbonylamino-2-octanone. The structure will be confirmed with 1H-NMR and LC/MS.

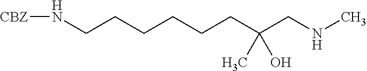

Step 2: (8-(Benzyloxycarbonylamino)-2-hydroxy-2-methyl-1-(methylamino)octane: (8-(benzyloxycarbonylamino)-2-hydroxy-2-methyl-1-((t-butoxycarbonyl)methylamino)octane will be dissolved 4N HCl in dioxane. After approximately 1 h, the solvents will be evaporated to dryness to give the product as its hydrochloride salt. The structure will be confirmed with LC/MS and ¹H-NMR.

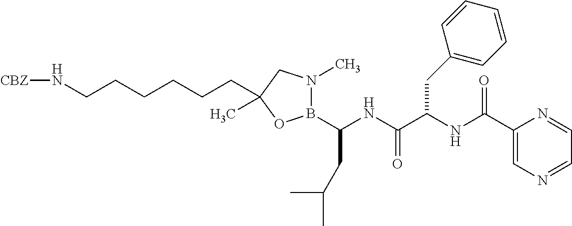

Step 3: Conjugate of bortezomib (8-(benzyloxycarbonylamino)-2-hydroxy-2-methyl-1-(methylamino)octane: In a manner similar to that described by Hebel et al. (J. Org. Chem. 2002, 67, 9452) bortezomib (1.0 mmol) will be dissolved in DMF and treated with a solution of (8-(benzyloxycarbonylamino)-2-hydroxy-2-methyl-1-(methylamino)octane (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

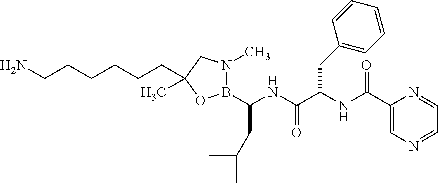

Step 4: Conjugate of bortezomib with (8-amino-2-hydroxy-2-methyl-1-(methylamino)octane: A 100-mL, round-bottom flask equipped with a magnetic stirrer will be charged with the conjugate of bortezomib (8-(benzyloxycarbonylamino)-2-hydroxy-2-methyl-1-(methylamino)octane [1 mmol], EtOAc (36 mL), and MeOH (0.5 mL). The mixture will be stirred for 5 min to afford a clear solution. 5% Pd/C (200 mg, 50% moisture) will be charged. The mixture will be evacuated for 1 min and then filled with H₂ with a balloon. The reaction will be stirred at ambient temperature for 3 h or until the reaction is complete. The mixture will be filtered through a Celite® pad to remove the catalyst; the combined filtrate concentrated and added into a suspension of Celite (10 g) in MTBE (300 mL) over 0.5 h with overhead stirring. The suspension will be filtered through a PP filter and the Celite®/product complex air-dried at ambient temperature for 16 h. It will be suspended in acetone (30 mL) with overhead stirring for 0.5 h and filtered. The filter cake will be washed with acetone (3×10 mL). The filtrate will be concentrated and added into cold water (300 mL) over 0.5 h with overhead stirring. The suspension will be stirred for another 0.5 h and filtered through a PP filter. The filter cake will be dried under vacuum for 24 h to afford product. The structure will be confirmed with 1H-NMR, HPLC and GPC.

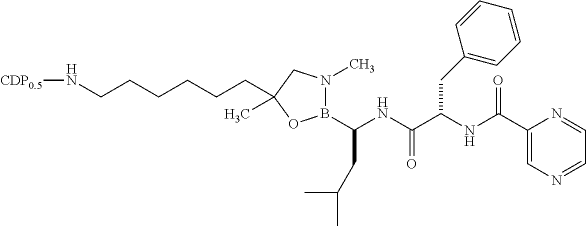

Step 5: Conjugate of bortezomib with (8-(CDP_{0.5}-carboxamido)-2-hydroxy-2-methyl-1-(methylamino)octane: A 100-mL round-bottom flask will be charged with the conjugate of bortezomib with (8-amino-2-hydroxy-2-methyl-1-(methylamino)octane (2.0 mmol per estimated number of cyclodextrin units in the CDP polymer) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with ¹H-NMR, HPLC and GPC.

### Example 3

### Synthesis of CDP conjugate with 1,2-Diol based boronic acid-Conjugate of bortezomib with (9-(CDP_{0.5}-carboxamido)-2,3-dihydroxy-2,3-dimethylnonane

Method A:

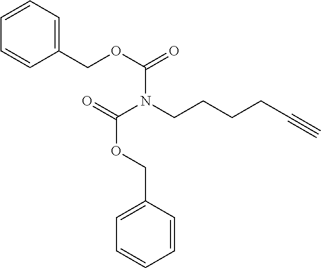

Step 1: 6-Bis-(benzyloxycarbonyl)amino-1-hexyne: 6-Chloro-1-hexyne (1.0 mmol) in THF will be treated with bis(benzyloxycarbonyl)amine (1.0 mmol) and potassium carbonate (1.2 mmol) in DMF (10 mL). After 16 h the reaction will be diluted with diethyl ether and washed successively with water, 1N hydrochloric acid and saturated sodium bicarbonate. After drying with sodium sulfate, the extract will be filtered and concentrated to give the crude product. This will be purified by chromatography. The structure will be confirmed with 1H-NMR and LC/MS.

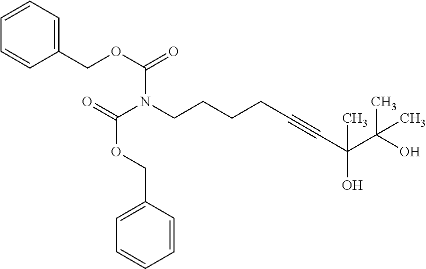

Step 2: 9-Bis-(benzyloxycarbonyl)amino-2,3-dihydroxy-2,3-dimethyl-4-nonyne: 6-Bis-(benzyloxycarbonyl)amino-1-hexyne (1.0 mmol) will be treated with lithium diisopropylamide in THF at −78° C. After 15 minutes, 3-hydroxy-3-methyl-2-butanone in THF will be added. After 1 hour at −78° C. the reaction will be quenched with saturated ammonium chloride solution and allowed to warm to room temperature. The reaction mixture will then be diluted with diethyl ether and successively washed with water, 1N hydrochloric acid, and saturated sodium bicarbonate. After drying with sodium sulfate, the extract will be filtered and the solvent evaporated to give the crude product. This will be purified by chromatography. The structure will be verified by 1H-NMR and LC/MS.

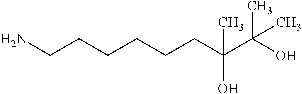

Step 3: 9-amino-2,3-dihydroxy-2,3-dimethylnonane: To a suspension of 10% Pd/C in methanol (˜1 g of catalyst per 1 g of substrate) in an appropriately sized flask will be added a solution of 9-bis-(benzyloxycarbonyl)amino-2,3-dihydroxy-2,3-dimethyl-4-nonyne in methanol. The flask will be evacuated and after 1 minute filled with hydrogen gas. After the reaction is complete the mixture will be filtered to remove the catalyst and the solvent evaporated to yield the title product. The structure will be verified by 1H-NMR and LC/MS.

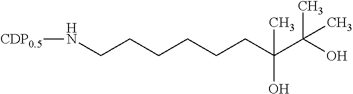

Step 4: 9-(CDP_{0.5}-carboxamido)-2,3-dihydroxy-2,3-dimethylnonane: A 100-mL round-bottom flask will be charged with 9-amino-2,3-dihydroxy-2,3-dimethylnonane (2.0 mmol per estimated number of cyclodextrin units in the CDP polymer) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

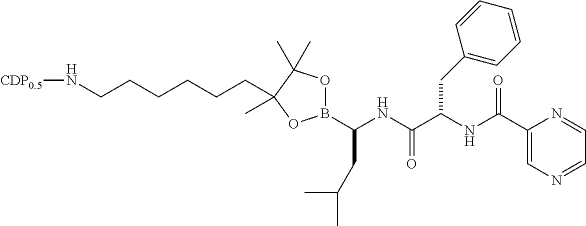

Step 5: Conjugate of bortezomib with 9-(CDP₀₅-carboxamido)-2,3-dihydroxy-2,3-dimethylnonane: In a manner similar to that described by Hebel et al. (J. Org. Chem. 2002, 67, 9452) bortezomib (2.0 mmol per estimated number of cyclodextrin units in the CDP polymer) will be dissolved in DMF and treated with a solution of 9-(CDP_{0.5}-carboxamido)-2,3-dihydroxy-2,3-dimethylnonane (1 g) in DMF and 4 Å MS. After 6 h at room temperature, the reaction mixture will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

Method B:

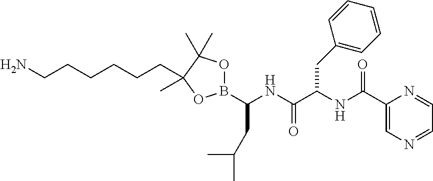

Step 1: Conjugate of bortezomib with 9-amino-2,3-dihydroxy-2,3-dimethylnonane: In a manner similar to that described by Hebel et al. (J. Org. Chem. 2002, 67, 9452) bortezomib (1.0 mmol) will be dissolved in DMF and treated with a solution of 9-amino-2,3-dihydroxy-2,3-dimethylnonane (from Method A, Step 3) (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1 H-NMR and LC/MS.

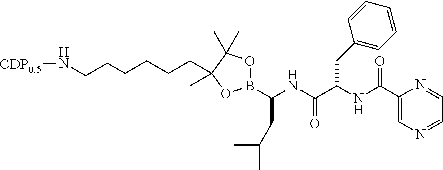

Step 2: Conjugate of bortezomib with 9-(CDP_{0.5}-carboxamido)-2,3-dihydroxy-2,3-dimethylnonane: A 100-mL round-bottom flask will be charged with the conjugate of bortezomib with 9-amino-2,3-dihydroxy-2,3-dimethylnonane (2.0 mmol per estimated number of cyclodextrin units in the CDP polymer) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with ¹H-NMR, HPLC and GPC.

### Example 4

### Synthesis of CDP conjugate with 1,3-Diol based boronic acid—Conjugate of bortezomib with (6-(CDP_{0.5}-carboxamido)-1-hydroxy-2-(hydroxymethyl)hexane

Method A:

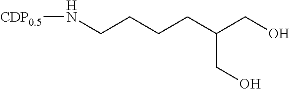

Step 1: 6-(CDP_{0.5}-carboxamido)-1-hydroxy-2-(hydroxymethyl)hexane: A 100-mL round-bottom flask will be charged with 6-amino-1-hydroxy-2-(hydroxymethyl)hexane (2.0 mmol per estimated number of cyclodextrin units in the CDP polymer) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

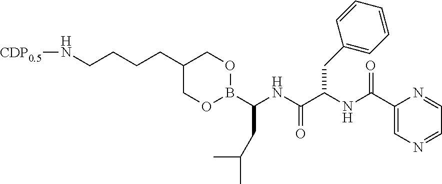

Step 2: Conjugate of bortezomib with (6-(CDP-carboxamido)-1-hydroxy-2-(hydroxymethyl)hexane: In a manner similar to that described by Hebel et al. (J. Org. Chem. 2002, 67, 9452) bortezomib (2.0 mmol per estimated number of cyclodextrin units in the CDP polymer) will be dissolved in DMF and treated with a solution of 6-(CDP_{0.5}-carboxamido)-1-hydroxy-2-(hydroxymethyl)hexane (1 g) in DMF and 4 Å MS. After 6 h at room temperature, the reaction mixture will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

Method B:

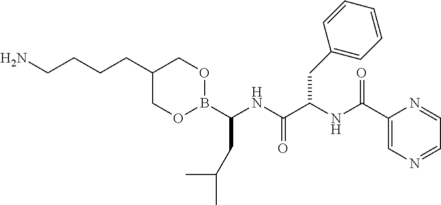

Step 1: Conjugate of bortezomib with 6-amino-1-hydroxy-2-(hydroxymethyl)hexane: In a manner similar to that described by Hebel et al. (J. Org. Chem. 2002, 67, 9452) bortezomib (1.0 mmol) will be dissolved in DMF and treated with a solution of 6-amino-1-hydroxy-2-(hydroxymethyl)hexane (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

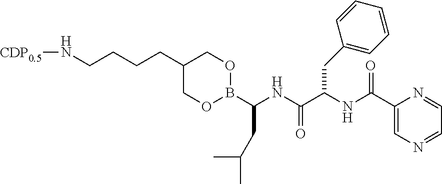

Step 2: Conjugate of bortezomib with 6-(CDP_{0.5}-carboxamido)-1-hydroxy-2-(hydroxymethyl)hexane: A 100-mL round-bottom flask will be charged with the conjugate of bortezomib with 6-amino-1-hydroxy-2-(hydroxymethyl)hexane (2.0 mmol per estimated number of cyclodextrin units in the CDP polymer) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with ¹H-NMR, HPLC and GPC.

### Example 5

### Synthesis of CDP Conjugate with Diethanolamine Based Boronic Acid—Conjugate of bortezomib with [(6-(CDP_{0.5}-carboxamidohexyl)-bis-(2-hydroxyethyl]amine

Method A:

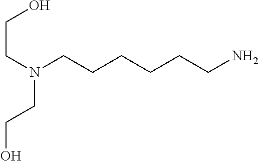

Step 1: Bis-(2-hydroxyethyl)hexylamine: In the manner described by R. M. Peck et al. (J. Am. Chem. Soc. 1959, 81, 3984) the title compound will be prepared.

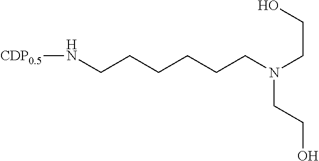

Step 2: Bis-(2-hydroxyethyl)-[(6-(CDP₀₅-carboxamidohexyl)amine: A 100-mL round-bottom flask will be charged with bis-(2-hydroxyethyl)hexylamine (2.0 mmol per estimated number of cyclodextrin units in the CDP polymer) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

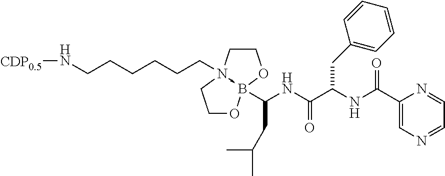

Step 3: Conjugate of bortezomib with bis-(2-hydroxyethyl)-[(6-(CDP_{0.5}-carboxamidohexyl)amine: In a manner similar to that described by Hebel et al. (J. Org. Chem. 2002, 67, 9452) bortezomib (2.0 mmol per estimated number of cyclodextrin units in the CDP polymer) will be dissolved in DMF and treated with a solution of bis-(2-hydroxyethyl)-[(6-(CDP_{0.5}-carboxamidohexyl)amine (1 g) in DMF and 4 Å MS. After 6 h at room temperature, the reaction mixture will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

Method B:

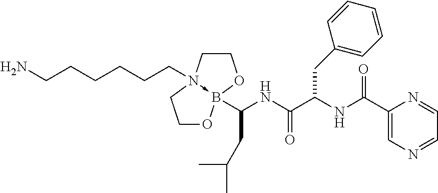

Step 1: Conjugate of bortezomib with bis-(2-hydroxyethyl)hexylamine: In a manner similar to that described by Hebel et al. (J. Org. Chem. 2002, 67, 9452) bortezomib (1.0 mmol) will be dissolved in DMF and treated with a solution of bis-(2-hydroxyethyl)hexylamine (from Method A, Step 1) (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

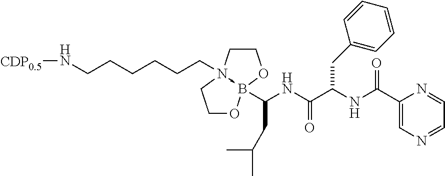

Step 2: Conjugate of bortezomib with bis-(2-hydroxyethyl)-[(6-(CDP_{0.5}-carboxamidohexyl)amine: A 100-mL round-bottom flask will be charged with the conjugate of bortezomib with bis-(2-hydroxyethyl)hexylamine (2.0 mmol) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

### Example 6

### Synthesis of CDP Conjugate of Iminodiacetic Acid Based Boronic Acid—Conjugate of bortezomib with [(6-(CDP_{0.5}-carboxamidohexyl)-carboxymethylamino]-acetate

Method A:

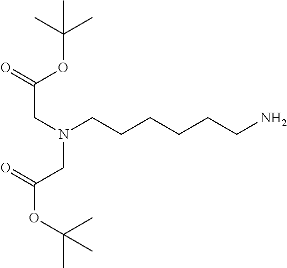

Step 1: t-Butyl-[(6-aminohexyl)-t-butoxycarbonylmethylamino]-acetate hydrochloride: In a manner similar to that described by M. Kruppa et al. (J. Am. Chem. Soc. 2005, 127, 3362) N—CBZ-1,6-diamino-hexane (4.9 mmol) will be dissolved in MeCN (20 ml) and mixed with t-butyl bromoacetate (10.6 mmol), potassium carbonate (2.92 g, 21.1 mmol) and a spatula tip of potassium iodide. The suspension will be stirred 2 days at 60° C. and monitored by TLC (ethyl acetate). The mixture will be filtrated, diluted with water and extracted with ethyl acetate. After drying over sodium sulfate the organic solvents will be evaporated to yield the crude product. Purification using column chromatography will give the CBZ-protected iminodiacetic acid-intermediate.

To deprotect the CBZ-group, the purified product will be hydrogentated over 10% Pd on carbon (50 wt. %) in methanol for 3 h. After completion of the reaction, the catalyst will be removed by filtration and the filtrate evaporated to dryness to give the title product. The structure will be confirmed with LC/MS and 1H-NMR.

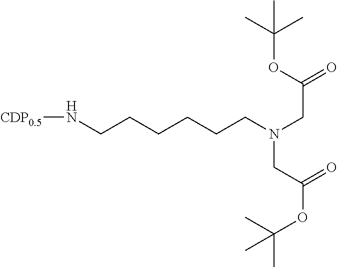

Step 2: t-Butyl-[(6-(CDP_{0.5}-carboxamidohexyl)-t-butoxycarbonylmethylamino]-acetate: A 100-mL round-bottom flask will be charged with t-butyl-[(6-aminohexyl)-t-butoxycarbonylmethylamino]-acetate hydrochloride (2.0 mmol per estimated number of cyclodextrin units in the CDP polymer) and DMF (5 mL). The mixture will be stirred for 15 min to afford a clear solution. CDP (1 g) and DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

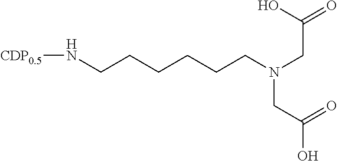

Step 3: [(6-(CDP_{0.5}-carboxamidohexyl)-carboxymethylamino]-acetate: A round-bottom flask equipped with a magnetic stirrer will be charged with t-butyl-[(6-(CDP_{0.5}-carboxamidohexyl)-t-butoxycarbonylmethylamino]-acetate, CH2Cl2 (5 volumes), and TFA (5 volumes). The reaction will be stirred at ambient temperature for 1 h or until the reaction is complete. The reaction will be concentrated and added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with ¹H-NMR, HPLC and GPC.

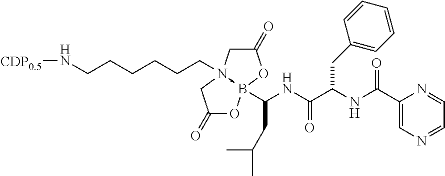

Step 4: Conjugate of bortezomib with [(6-(CDP_{0.5}-carboxamidohexyl)-carboxymethylamino]-acetate: In a manner similar to that described by Hebel et al. (J. Org. Chem. 2002, 67, 9452) bortezomib (2.0 mmol per estimated number of cyclodextrin units in the CDP polymer) will be dissolved in DMF and treated with a solution of [(6-(CDP_{0.5}-carboxamidohexyl)-carboxymethylamino]-acetate (1 g) in DMF and 4 Å MS. After 6 h at room temperature, the reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

Method B:

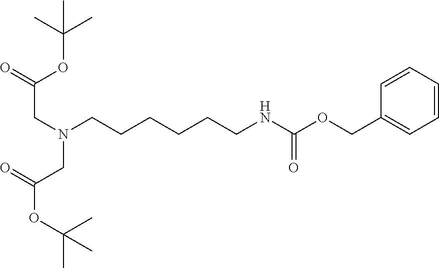

Step 1: tert-Butyl-[(6-benzyloxycarbonylaminohexyl)-tert-butoxycarbonylmethylamino]-acetate: In the manner described by M. Kruppa et al. (J. Am. Chem. Soc. 2005, 127, 3362) the title compound will be produced.

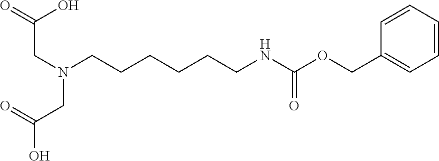

Step 2: [(6-Benzyloxycarbonylaminohexyl)-carboxymethylamino]-acetate: To a solution of tert-butyl-[(6-benzyloxycarbonylaminohexyl)-tert-butoxycarbonylmethylamino]-acetate in dichloromethane will be added at 0° C. trifluoroacetic acid. After 1 hour the solvent will be evaporated to yield the title product. The structure will be confirmed with 1H-NMR and LC/MS.

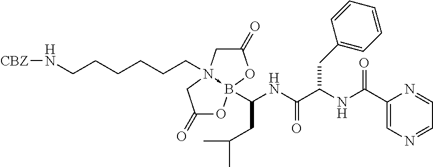

Step 3: Conjugate of bortezomib with [(6-(benzyloxycarbonylaminohexyl)-carboxymethylamino]-acetate: In a manner similar to that described by Hebel et al. (J. Org. Chem. 2002, 67, 9452) bortezomib (1.0 mmol) will be dissolved in DMF and treated with a solution of [(6-benzyloxycarbonylaminohexyl)-carboxymethylamino]-acetate (1.0 mmol) in DMF and 4 Å MS. After 6 h at room temperature, the reaction mixture will be added into in MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

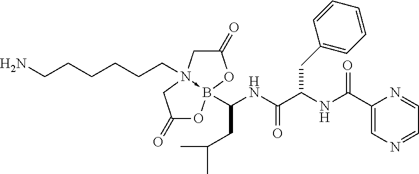

Step 4: Conjugate of bortezomib with [(6-(aminohexyl)-carboxymethylamino]-acetate: A 100-mL, round-bottom flask equipped with a magnetic stirrer will be charged with the conjugate of bortezomib with [(6-(benzyloxycarbonylaminohexyl)-carboxymethylamino]-acetate [1.06 mmol], EtOAc (36 mL), and MeOH (0.5 mL). The mixture will stirred for 5 min to afford a clear solution. 5% Pd/C (200 mg, 50% moisture) will be charged. The mixture will be evacuated for 1 min and then filled with H₂ with a balloon. The reaction will be stirred at ambient temperature for 3 h or until the reaction is complete. The mixture will be added to MTBE (30 mL) over 0.5 h with overhead stirring. The suspension will be stirred for another 0.5 h and filtered through a PP filter. The filter cake will be dried under vacuum for 24 h to afford product. The structure will be confirmed with 1H-NMR and LC/MS.

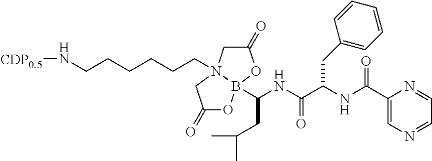

Step 5: Conjugate of bortezomib with [(6-(CDP_{0.5}-carboxamidohexyl)-carboxymethylamino]-acetate: A 100-mL round-bottom flask will be charged with the conjugate of bortezomib with [(6-(aminohexyl)-carboxymethylamino]-acetate (2.0 mmol per estimated number of cyclodextrin units in the CDP polymer) and DMF (5 mL). The mixture will be stirred for 15 min to afford a clear solution. CDP (1 g) in DMF (20 mL) will be added and the mixture stirred for 10 min. EDC.HCl (2.3 mmol per estimated number of cyclodextrin units in the CDP polymer), DMAP (1.0 mmol per estimated number of cyclodextrin units in the CDP polymer), and TEA (5.0 mmol per estimated number of cyclodextrin units in the CDP polymer) will be added and the reaction stirred at ambient temperature for 6 h or until completion of the reaction. The reaction will be added into acetone or a mixture of acetone and diethylether or MTBE. The resulting precipitate will be isolated by filtration or decantation of the supernatant. The precipitate will then be dissolved in water and dialyzed for 3 days with a 25 kDa MWCO. The lyophilized solution will be filtered through a 2 μM filter and the filtrate lyophilized to give the title product. The structure will be confirmed with 1H-NMR, HPLC and GPC.

The CDP polymer used in Examples 1-6 can be any CDP polymer described herein that has two functional groups, such as —COOH, that would react with an amino group. In one embodiment, the CDP polymer is represented by the following structural formula:

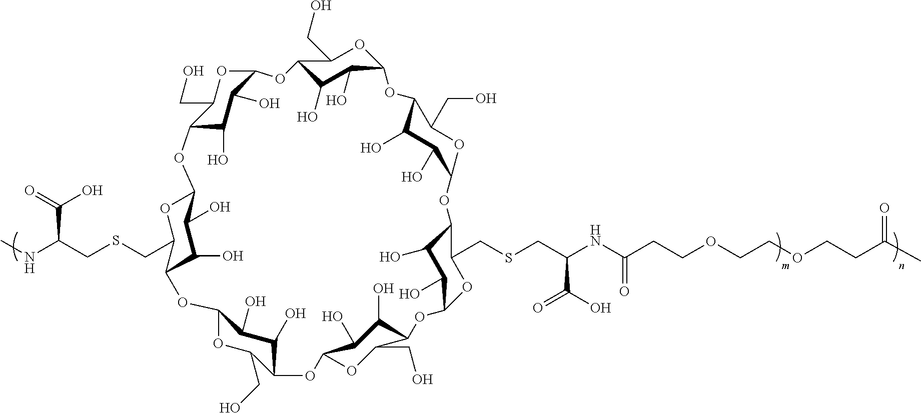

A CDP-proteasome inhibitor conjugate comprising a boronic acid containing proteasome inhibitor described herein other than bortezomib can be prepared in similar manners as described in Example 1-6 with suitable starting materials.

Other embodiments are in the claims.

1. A nanoparticle comprising a CDP-proteasome inhibitor conjugate comprising a plurality of proteasome inhibitor molecules coupled with a CDP moiety.

2. The nanoparticle of claim 1, wherein the nanoparticle has a conjugate number of 1 to 25.

3. The nanoparticle of claim 1, wherein the nanoparticle has a conjugate number of 1 to 5.

4. The nanoparticle of claim 1, wherein the CDP-proteasome inhibitor conjugate is selected from the group consisting of: 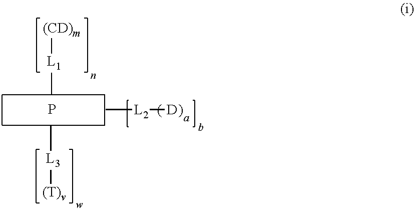
wherein
P represents a linear or branched polymer chain;
CD represents a cyclodextrin moiety;
L₁, L₂ and L₃, independently for each occurrence are optional linker groups;
D, independently for each occurrence, is a proteasome inhibitor;
T, independently for each occurrence, is a targeting ligand or precursor thereof;
a, m, and v, independently for each occurrence, are integers in the range of 1 to 10;
n and w, independently for each occurrence, are integers in the range of 0 to 30,000; and
b represents an integer in the range of 1 to about 30,000;
wherein either P comprises cyclodextrin moieties or n is at least 1; 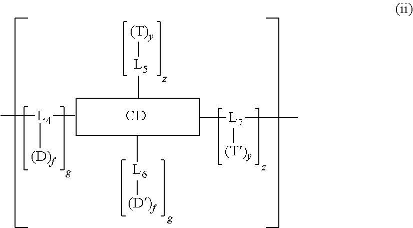
wherein
CD represents a cyclodextrin moiety, or derivative thereof;
L₄, L₅, L₆, and L₇, independently for each occurrence are optional linker groups;
D and D′, independently for each occurrence, is a proteasome inhibitor;
T and T′, independently for each occurrence, are targeting ligands or precursor thereof;
f and y, independently for each occurrence, are integers in the range from 1 to 10; and
g and z, independently for each occurrence, are integers in the range from 0 to 10; 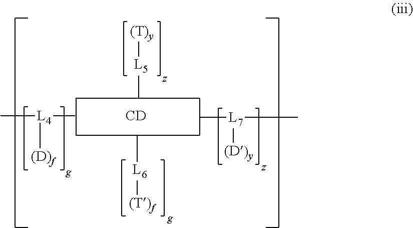
wherein
CD represents a cyclodextrin moiety, or derivative thereof;
L₄, L₅, L₆, and L₇, independently for each occurrence are optional linker groups;
D and D′, independently for each occurrence, is a proteasome inhibitor;
T and T′, independently for each occurrence, are targeting ligands or precursor thereof;
f and y, independently for each occurrence, are integers in the range from 1 to 10; and
g and z, independently for each occurrence, are integers in the range from 0 to 10; 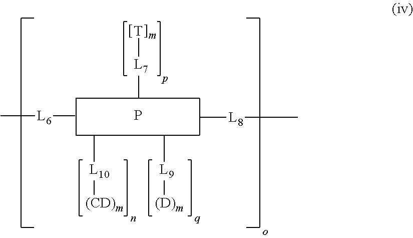
wherein
P represents a monomer unit of a polymer that comprises cyclodextrin moieties;
T, independently for each occurrence, is a targeting ligand or a precursor thereof;
L₆, L₇, L₈, L₉, and L₁₀, independently for each occurrence, are optional linker groups;
CD, independently for each occurrence, is a cyclodextrin moiety or a derivative thereof;
D, independently for each occurrence, is a proteasome inhibitor;
m, independently for each occurrence, is an integer in the range from 1 to 10;
o represents an integer in the range of 1 to about 30,000; and
p, n, and q, independently for each occurrence, are integers in a range from 0 to 10; and
wherein CD and D are each present at least once; 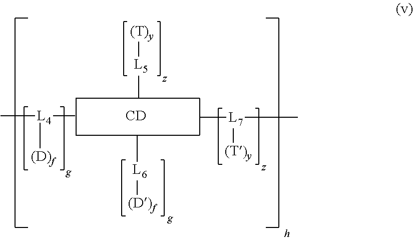
wherein
CD is a cyclodextrin moiety or a derivative thereof;
L₄, L₅, L₆, and L₇, independently for each occurrence, are optional linker groups;
D and D′, independently for each occurrence, is a proteasome inhibitor;
T and T′, independently for each occurrence, is a targeting ligand or a precursor thereof;
f and y, independently for each occurrence, are integers in a range from 1 to 10;
g and z, independently for each occurrence, are integers in a range from 0 to 10; and
h is an integer in a range from 1 and 30,000;
wherein at least one occurrence of g represents an integer greater than 0; and 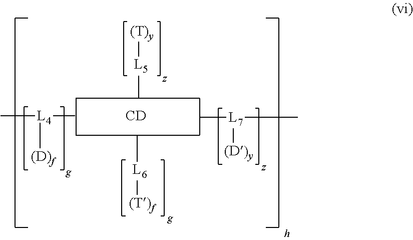
wherein
CD is a cyclodextrin moiety or a derivative thereof;
L₄, L₅, L₆, and L₇, independently for each occurrence, are optional linker groups;
D and D′, independently for each occurrence, is a proteasome inhibitor;
T and T′, independently for each occurrence, is a targeting ligand or a precursor thereof;
f and y, independently for each occurrence, are integers in a range from 1 to 10;
g and z, independently for each occurrence, are integers in a range from 0 to 10; and
h is an integer in a range from 1 and 30,000;
wherein at least one occurrence of g represents an integer greater than 0.

5. The nanoparticle of claim 4, wherein the proteasome inhibitor D and/or D′ is a boronic acid-containing compound.

6. The nanoparticle of claim 4, wherein the proteasome inhibitor D and/or is bortezomib.

7. The nanoparticle of claim 1, wherein the CDP-proteasome inhibitor conjugate is a conjugate of formula: 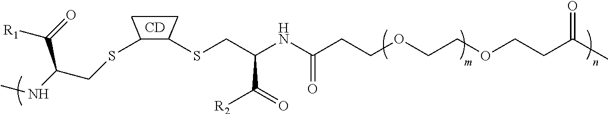
wherein R₁ and R₂ are independently selected from OH and a boronic acid-containing proteasome inhibitor, wherein the boronic acid-containing proteasome inhibitor is optionally linker-bound;
m is an integer such that the co-monomer 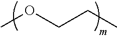
having a molecular weight in from about 2,000 to about 5,000 Da;
n is an integer from 4 to 20; and
CD is alpha-cyclodextrin, beta-cyclodextrin, or gamma-cyclodextrin.

8. The nanoparticle of claim 7, wherein the co-monomer 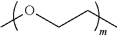
has a molecular weight from about 2,000 to about 3,000 Da.

9. The nanoparticle of claim 7, wherein the linker is a linker set forth in Table 2.

10. The nanoparticle of claim 7, wherein R₁ and R₂ are independently selected from OH and a linker-bound drug substance of formula: 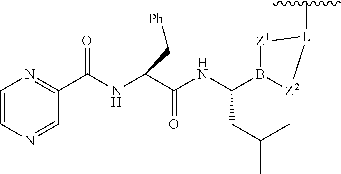
wherein Z¹ and Z² are bonds between boron and linker L.

11. A pharmaceutical composition comprising the nanoparticle of claim 1.

12. The pharmaceutical composition of claim 11, wherein at least 60% of the nanoparticles in the pharmaceutical composition have a conjugate number from 1-5.

13. A method of treating a proliferative disorder comprising administering to a patient in need thereof a therapeutically effective amount of the pharmaceutical composition of claim 11.

14. (canceled)
